(19) United States
(12) Patent Application Publication
(10) Pub. No.: US 2021/0402210 A1
(43) Pub. Date: Dec. 30, 2021

(54) MULTI-CHANNEL BIOACTIVE LIGHTING

(71) Applicant: Ecosense Lighting Inc., Los Angeles, CA (US)

(72) Inventors: Raghuram L. V. Petluri, Los Angeles, CA (US); Paul Kenneth PICKARD, Los Angeles, CA (US); Benjamin Harrison, Los Angeles, CA (US)

(21) Appl. No.: 17/316,362

(22) Filed: May 10, 2021

Related U.S. Application Data
(63) Continuation of application No. PCT/US2019/060634, filed on Nov. 8, 2019.
Application No. PCT/US2019/060634 is a continuation-in-part of application No. 16/393,660, filed on Apr. 24, 2019, now Pat. No. 10,805,998.
Application No. 16/393,660 is a continuation-in-part of application No. PCT/US2019/013380, filed on Jan. 11, 2019.
Application No. PCT/US2019/013380 is a continuation-in-part of application No. PCT/US2019/013359, filed on Jan. 11, 2019.
Application No. PCT/US2019/013359 is a continuation-in-part of application No. PCT/US2019/013379, filed on Jan. 11, 2019.
Application No. PCT/US2019/013379 is a continuation-in-part of application No. PCT/US2019/013356, filed on Jan. 11, 2019.
(60) Provisional application No. 62/757,664, filed on Nov. 8, 2018.
(60) Provisional application No. 62/757,672, filed on Nov. 8, 2018.
(60) Provisional application No. 62/758,411, filed on Nov. 9, 2018.
(60) Provisional application No. 62/758,447, filed on Nov. 9, 2018.
(60) Provisional application No. 62/885,162, filed on Aug. 9, 2019.

(51) Int. Cl. A61N 5/06; H05B 45/20

(52) U.S. Cl. CPC A61N 5/0618; H05B 45/20; A61N 2005/0626; A61N 2005/0659; A61N 2005/0652; A61N 2005/0663

(57) ABSTRACT

The present disclosure provides bioactive lighting systems suitable for generating white light. The lighting systems has lighting channels configured to produce white light having a color point and a spectral power distribution. The combination of emissions from selected channels results in white light having adjusted color points. The system is controlled to selectively increase or decrease CSE and to selectively provide LRNE.

### CROSS-REFERENCE TO RELATED APPLICATIONS

This application claims the benefit of International Patent Application No. PCT/US2019/013356 filed Jan. 11, 2019, which claims the benefit of U.S. Provisional Patent Application No. 62/757,664 filed Nov. 8, 2018; International Patent Application No. PCT/US2019/013359 filed Jan. 11, 2019, which claims the benefit of U.S. Provisional Patent Application No. 62/757,672 filed Nov. 8, 2018; U.S. patent application Ser. No. 16/393,660 filed Apr. 24, 2019, which is a Continuation of International Patent Application No. PCT/US2019/013380 filed Jan. 11, 2019, which claims the benefit of U.S. Provisional Patent Application No. 62/758,411 filed Nov. 9, 2018; International Patent Application No. PCT/US2019/013379 filed Jan. 11, 2019, which claims the benefit of U.S. Provisional Patent Application No. 62/758,447 filed Nov. 9, 2018; and U.S. Provisional Patent Application No. 62/885,162 filed Aug. 9, 2019, the entire contents of which are incorporated by reference as if fully set forth herein.

### FIELD OF THE DISCLOSURE

This disclosure is in the field of bioactive solid-state lighting. In particular, the disclosure relates to devices for use in, and methods of, providing tunable bioactive white light with high color rendering performance and controllable biological effects.

### BACKGROUND

A wide variety of light emitting devices are known in the art including, for example, incandescent light bulbs, fluorescent lights, and semiconductor light emitting devices such as light emitting diodes (“LEDs”).

There are a variety of resources utilized to describe the light produced from a light emitting device, one commonly used resource is 1931 CIE (Commission Internationale de l'Éclairage) Chromaticity Diagram. The 1931 CIE Chromaticity Diagram maps out the human color perception in terms of two CIE parameters x and y. The spectral colors are distributed around the edge of the outlined space, which includes all of the hues perceived by the human eye. The boundary line represents maximum saturation for the spectral colors, and the interior portion represents less saturated colors including white light. The diagram also depicts the Planckian locus, also referred to as the black body locus (BBL), with correlated color temperatures, which represents the chromaticity coordinates (i.e., color points) that correspond to radiation from a black-body at different temperatures. Illuminants that produce light on or near the BBL can thus be described in terms of their correlated color temperatures (CCT). These illuminants yield pleasing “white light” to human observers, with general illumination typically utilizing CCT values between 1,800K and 10,000K.

Color rendering index (CRI) is described as an indication of the vibrancy of the color of light being produced by a light source. In practical terms, the CRI is a relative measure of the shift in surface color of an object when lit by a particular lamp as compared to a reference light source, typically either a black-body radiator or the daylight spectrum. The higher the CRI value for a particular light source, the better that the light source renders the colors of various objects it is used to illuminate.

Color rendering performance may be characterized via standard metrics known in the art. Fidelity Index (Rf) and the Gamut Index (Rg) can be calculated based on the color rendition of a light source for 99 color evaluation samples (“CES”). The 99 CES provide uniform color space coverage, are intended to be spectral sensitivity neutral, and provide color samples that correspond to a variety of real objects. Rf values range from 0 to 100 and indicate the fidelity with which a light source renders colors as compared with a reference illuminant. In practical terms, the Rf is a relative measure of the shift in surface color of an object when lit by a particular lamp as compared to a reference light source, typically either a black-body radiator or the daylight spectrum. The higher the Rf value for a particular light source, the better that the light source renders the colors of various objects it is used to illuminate. The Gamut Index Rg evaluates how well a light source saturates or desaturates the 99 CES compared to the reference source.

LEDs have the potential to exhibit very high power efficiencies relative to conventional incandescent or fluorescent lights. Most LEDs are substantially monochromatic light sources that appear to emit light having a single color. Thus, the spectral power distribution of the light emitted by most LEDs is tightly centered about a “peak” wavelength, which is the single wavelength where the spectral power distribution or “emission spectrum” of the LED reaches its maximum as detected by a photo-detector. LEDs typically have a full-width half-maximum wavelength range of about 10 nm to 30 nm, comparatively narrow with respect to the broad range of visible light to the human eye, which ranges from approximately from 380 nm to 800 nm.

In order to use LEDs to generate white light, LED lamps have been provided that include two or more LEDs that each emit a light of a different color. The different colors combine to produce a desired intensity and/or color of white light. For example, by simultaneously energizing red, green and blue LEDs, the resulting combined light may appear white, or nearly white, depending on, for example, the relative intensities, peak wavelengths and spectral power distributions of the source red, green and blue LEDs. The aggregate emissions from red, green, and blue LEDs typically provide poor color rendering for general illumination applications due to the gaps in the spectral power distribution in regions remote from the peak wavelengths of the LEDs.

White light may also be produced by utilizing one or more luminescent materials such as phosphors to convert some of the light emitted by one or more LEDs to light of one or more other colors. The combination of the light emitted by the LEDs that is not converted by the luminescent material(s) and the light of other colors that are emitted by the luminescent material(s) may produce a white or near-white light.

LED lamps have been provided that can emit white light with different CCT values within a range. Such lamps utilize two or more LEDs, with or without luminescent materials, with respective drive currents that are increased or decreased to increase or decrease the amount of light emitted by each LED. By controllably altering the power to the various LEDs in the lamp, the overall light emitted can be tuned to different CCT values. The range of CCT values that can be provided with adequate color rendering values and efficiency is limited by the selection of LEDs.

The spectral profiles of light emitted by white artificial lighting can impact circadian physiology, alertness, and cognitive performance levels. Bright artificial light can be used in a number of therapeutic applications, such as in the treatment of seasonal affective disorder (SAD), certain sleep problems, depression, jet lag, sleep disturbances in those with Parkinson's disease, the health consequences associated with shift work, and the resetting of the human circadian clock. Artificial lighting may change natural processes, interfere with melatonin production, or disrupt the circadian rhythm. Blue light may have a greater tendency than other colored light to affect living organisms through the disruption of their biological processes which can rely upon natural cycles of daylight and darkness. Exposure to blue light late in the evening and at night may be detrimental to one's health. Some blue or royal blue light within lower wavelengths can have hazardous effects to human eyes and skin, such as causing damage to the retina.

Significant challenges remain in providing LED lamps that can provide white light across a range of CCT values while simultaneously achieving high efficiencies, high luminous flux, good color rendering, and acceptable color stability. It is also a challenge to provide lighting apparatuses that can provide desirable lighting performance while allowing for the control of circadian energy performance.

### DISCLOSURE

The present disclosure provides aspects of lighting systems comprising a first bioactive lighting channel configured to produce a first white light having a first color point and a first spectral power distribution, a second bioactive lighting channel configured to produce a second white light having a second color point and a second spectral power distribution, and a control system configured to independently change the intensity of each of the first lighting channel and the second lighting channel. In some implementations, the first white light and second white light combined together can form a third white light having a third color point and a third spectral power distribution. In some implementations, the control system can be further configured to change the intensity of each of the first lighting channel and the second lighting channel to provide the third white light with the third color point at a plurality of points along a predefined path near the black body locus in the 1931 CIE Chromaticity Diagram between and including both the first color point and the second color point. In certain implementations, the first spectral power distribution can have a first circadian-stimulating energy characteristic, the second spectral power distribution can have a second circadian-stimulating energy characteristic, and the third spectral power distribution can have a third circadian-stimulating energy characteristic. In some implementations, the third white light at each of the plurality of points along the predefined path can have an Ra value greater than or equal to 80. In some implementations, the first color point can have a CCT between about 4000K and about 6500K. In further implementations, the second color point can have a CCT between about 2700K and about 1800K. The first lighting channels can have LEDs having an emission with a first peak wavelength of between about 440 nm and about 510 nm. The second lighting channels can have LEDs having an emission with a second peak wavelength of between about 380 nm and about 420 nm. In yet further implementations, the lighting systems can have one or more LRNE lighting channels configured to provide Visible LRNE, Non-Visible LRNE, or both Visible and Non-Visible LRNE.

In some aspects, the present disclosure provides methods of generating white light, the methods comprising producing a first white light, a second white light, or a combination of the first white light and the second white light, wherein the first white light is produced from a first lighting channel of a lighting system, the first white light having a first color point and a first spectral power distribution, wherein the second white light is produced from a second lighting channel of the lighting system, the second white light having a second color point and a second spectral power distribution, with the methods further comprising combining the first white light, the second white light, or the combination of the first white light and the second white light to form a third white light having a third color point and a third spectral power distribution, with the methods further comprising changing the intensity of each of the first lighting channel and the second lighting channel with a control system to provide the third white light with the third color point at a plurality of points along a predefined path near the black body locus in the 1931 CIE Chromaticity Diagram between and including both the first color point and the second color point, wherein the first spectral power distribution has a first circadian-stimulating energy characteristic, the second spectral power distribution has a second circadian-stimulating energy characteristic, and the third spectral power distribution has a third circadian-stimulating energy characteristic. In some implementations, the third white light at each of the plurality of points along the predefined path can have an Ra value greater than or equal to 80. In some implementations, the first color point can have a CCT between about 4000K and about 6500K. In some implementations, the second color point can have a CCT between about 2700K and about 1800K. The first lighting channels can have LEDs having an emission with a first peak wavelength of between about 440 nm and about 510 nm. The second lighting channels can have LEDs having an emission with a second peak wavelength of between about 380 nm and about 420 nm.

In some aspects, the present disclosure provides methods of generating white light with the devices described herein by providing a first bioactive lighting channel configured to produce a first white light having a first color point and a first spectral power distribution; a second lighting channel configured to produce a second white light having a second color point and a second spectral power distribution; a third bioactive lighting channel configured to produce a third white light having a third color point and a third spectral power distribution; and a control system configured to independently change the intensity of each of the first, second and third lighting channel; wherein at least two of the visible channels combined together to form white light having effecting bio-physiological functions; wherein the control system is further configured to change the intensity of each of the lighting channels to provide white light with a color point at a plurality of points along a predefined path near the black body locus in the 1931 CIE Chromaticity Diagram; and, wherein the first spectral power distribution has a first circadian-stimulating energy (CSE) characteristic, the second spectral power distribution has a second circadian-stimulating energy characteristic, and the third spectral power distribution has a long red near infrared energy (LRNE) energy characteristic.

In some instance the bioactive lighting has a lighting channel LED emission is with a peak wavelength of between 440 nm and about 510 nm. In some instance the bioactive lighting has a lighting channel with an LED emission having a peak wavelength of between 380 nm and 420 nm. In some instance the bioactive lighting has a LRNE lighting channel having an emission with a peak wavelength of between 625 nm and 1400 nm. In some instance the bioactive lighting has a LRNE lighting channel having an emission with a peak wavelength of between 625 nm and 700 nm. In some instance the bioactive lighting has a LRNE lighting channel having an emission with a peak wavelength of between 700 nm and 1000 nm. In some instance the bioactive lighting has a LRNE lighting channel having an emission with a peak wavelength of between 800 nm and 1400 nm.

In some aspects, the present disclosure provides methods of generating white light with the devices described herein by providing a first bioactive lighting channel configured to produce a first white light having a first color point and a first spectral power distribution; a second lighting channel configured to produce a second white light having a second color point and a second spectral power distribution; a third bioactive lighting channel configured to produce a third white light having a third color point and a third spectral power distribution; and a control system configured to independently change the intensity of each of the first, second and third lighting channel; wherein at least two of the visible channels combined together to form white light having effecting bio-physiological functions; wherein the control system is further configured to change the intensity of each of the lighting channels to provide white light with a color point at a plurality of points along a predefined path near the black body locus in the 1931 CIE Chromaticity Diagram; and, wherein the first spectral power distribution has a first circadian-stimulating energy (CSE) characteristic, the second spectral power distribution has a second circadian-stimulating energy characteristic, the third spectral power distribution has a long red near infrared energy (LRNE) energy characteristic and an additional non-visible LRNE having an emission with a peak wavelength of between above 700 nm and 1400 nm. In some instances the emission produced by the additional non-visible LRNE is controlled independently by the control system of the visible channels.

In some aspects, the present disclosure provides methods of providing aliquots of bioactive lighting, the method including providing light from a first LED configured to produce a circadian-stimulating energy (CSE) characteristic; providing light from a second LED configured to produce a LRNE characteristic; and, providing a control system configured to independently change the duration of emission and intensity of each aliquot of the CSE and LRNE light. In some instances at least one of the LRNE and CSE effect bio-physiological functions.

In some aspects, the present disclosure provides methods of providing aliquots of bioactive lighting, the method including providing light from a first LED configured to produce a circadian-stimulating energy (CSE) characteristic; providing light from a second LED configured to produce a LRNE characteristic; and, providing a control system configured to independently change the duration of emission and intensity of each aliquot of the CSE and LRNE light. In some instances a third LED which is non-visible LRNE having an emission with a peak wavelength of between above 700 nm and 1400 nm is added. In some instances the second LED has a peak wavelength of between above 625 and 700 nm. In some instances the control system provides at least one aliquot of LRNE and CSE at a duration of between about 10 ms and 100 ms, In some instances the frequency of one or more of the CSE or LRNE is between 10 Hz and 0.5 mHz and vary from a single pulse to 400,000 pulses.

The general disclosure and the following further disclosure are exemplary and explanatory only and are not restrictive of the disclosure, as defined in the appended claims. Other aspects of the present disclosure will be apparent to those skilled in the art in view of the details as provided herein. In the figures, like reference numerals designate corresponding parts throughout the different views. All callouts and annotations are hereby incorporated by this reference as if fully set forth herein.

### DRAWINGS

The summary, as well as the following detailed description, is further understood when read in conjunction with the appended drawings. For the purpose of illustrating the disclosure, there are shown in the drawings exemplary implementations of the disclosure; however, the disclosure is not limited to the specific methods, compositions, and devices disclosed. In addition, the drawings are not necessarily drawn to scale. In the drawings:

FIG. 1A illustrates aspects of lighting systems according to the present disclosure;

FIG. 1B illustrates aspects of lighting systems according to the present disclosure;

FIG. 1C illustrates aspects of lighting systems according to the present disclosure;

FIG. 2 illustrates aspects of lighting systems according to the present disclosure;

FIG. 3 depicts a graph of a 1931 CIE Chromaticity Diagram illustrating the location of the Planckian locus;

FIG. 4 depicts a graph of aspects of lighting systems of the present disclosure and commercially available lighting systems;

FIG. 5 illustrates some aspects of lighting systems according to the present disclosure;

FIG. 6 illustrates some aspects of lighting systems according to the present disclosure;

FIG. 7 illustrates some aspects of lighting systems according to the present disclosure, including some suitable color ranges for light generated by components of the devices;

FIG. 8 illustrates some aspects of lighting systems according to the present disclosure;

FIG. 9 illustrates some aspects of lighting systems according to the present disclosure, including aspects of spectral power distributions for light generated by components of the devices;

FIG. 10 illustrates some aspects of lighting systems according to the present disclosure, including aspects of spectral power distributions for light generated by components of the devices;

FIG. 11 illustrates some aspects of lighting systems according to the present disclosure, including aspects of spectral power distributions for light generated by components of the devices;

FIG. 12 illustrates some aspects of lighting systems according to the present disclosure, including aspects of spectral power distributions for light generated by components of the devices;

FIG. 13 illustrates some aspects of lighting systems according to the present disclosure, including aspects of spectral power distributions for light generated by components of the devices;

FIG. 14 illustrates some aspects of lighting systems according to the present disclosure, including aspects of spectral power distributions for light generated by components of the devices;

FIG. 15 illustrates some aspects of lighting systems according to the present disclosure, including aspects of spectral power distributions for light generated by components of the devices;

FIG. 16 illustrates some aspects of lighting systems according to the present disclosure, including aspects of spectral power distributions for light generated by components of the devices;

FIG. 17 illustrates some aspects of lighting systems according to the present disclosure, including aspects of spectral power distributions for light generated by components of the devices; and

FIG. 18 illustrates aspects of lighting systems according to the present disclosure.

FIG. 19 illustrates some aspects of LRNE spectral power distributions for light generated by components of lighting systems according to the present disclosure.

FIG. 20 illustrates some aspects of LRNE spectral power distributions for light generated by components of lighting systems according to the present disclosure.

FIG. 21 illustrates some aspects of LRNE spectral power distributions for light generated by components of lighting systems according to the present disclosure.

FIG. 22 illustrates some aspects of LRNE spectral power distributions for light generated by components of lighting systems according to the present disclosure.

FIG. 23 illustrates some aspects of LRNE spectral power distributions for light generated by components of lighting systems according to the present disclosure.

FIG. 24 illustrates some aspects of LRNE spectral power distributions for light generated by components of lighting systems according to the present disclosure.

FIG. 25 illustrates some aspects of LRNE spectral power distributions for light generated by components of lighting systems according to the present disclosure.

FIG. 26 illustrates some aspects of LRNE spectral power distributions for light generated by components of lighting systems according to the present disclosure.

FIG. 27 schematic view of lighting channels of systems including LRNE channels of the present disclosure.

FIG. 28 depicts aspects of control of the bioactive illumination.

FIG. 29 is a block diagram of computing systems and methods of control of bioactive illumination.

FIG. 30 is an overview of a bioactive illumination control systems and methods.

All descriptions and callouts in the Tables and Figures are hereby incorporated by this reference as if fully set forth herein.

### FURTHER DISCLOSURE

The present disclosure may be understood more readily by reference to the following detailed description taken in connection with the accompanying figures and examples, which form a part of this disclosure. It is to be understood that this disclosure is not limited to the specific devices, methods, applications, conditions or parameters described and/or shown herein, and that the terminology used herein is for the purpose of describing particular exemplars by way of example only and is not intended to be limiting of the claimed disclosure. Also, as used in the specification including the appended claims, the singular forms “a,” “an,” and “the” include the plural, and reference to a particular numerical value includes at least that particular value, unless the context clearly dictates otherwise. The term “plurality”, as used herein, means more than one. When a range of values is expressed, another exemplar includes from the one particular value and/or to the other particular value. Similarly, when values are expressed as approximations, by use of the antecedent “about,” it will be understood that the particular value forms another exemplar. All ranges are inclusive and combinable.

The term “circadian-stimulating energy characteristics” refers to any characteristics of a spectral power distribution that may have biological effects on a subject. In some aspects, the circadian-stimulating energy characteristics of aspects of the lighting systems of this disclosure can include one or more of CS, CLA, EML, BLH, CER, CAF, LEF, circadian power, circadian flux, and the relative amount of power within one or more particular wavelength ranges.

It is to be appreciated that certain features of the disclosure which are, for clarity, described herein in the context of separate exemplar, may also be provided in combination in a single exemplary implementation. Conversely, various features of the disclosure that are, for brevity, described in the context of a single exemplary implementation, may also be provided separately or in any sub combination. Further, reference to values stated in ranges include each and every value within that range.

In some aspects, the present disclosure provides lighting systems having a first lighting channel and a second lighting channel. The first lighting channels of the present disclosure can be configured to produce a first white light having a first color point and a first spectral power distribution. The second lighting channels of the present disclosure can be configured to produce a second white light having a second color point and a second spectral power distribution. The lighting systems can further include a control system that is configured to independently change the intensity of each of the first and second lighting channels. With different relative intensities of the first and second lighting channels, the lighting system can provide a combined light from combining the first white light and second white light together as a third white light having a third color point and a third spectral power distribution. In some implementations, one of the first and second lighting channels can be shut off completely, such that the third white light is the same as the other of the first and second lighting channels that is not shut off; in further implementations, the other channel can be shut off such that the third white light is the same as the other lighting channel. In some implementations the third white light can be switched from being the same as the first lighting channel and the same as the second lighting channel by alternately shutting off and turning on the first and second lighting channels. In further implementations, a plurality of third color points can be generated along the tie line between a particular pair of first color point and second color point of the first and second lighting channels on the 1931 CIE Chromaticity Diagram. In some implementations, the plurality of third color points can form a predefined path near the black body locus on the 1931 CIE Chromaticity Diagram. In certain implementations, the plurality of third color points can form a predefined path within a 7-step MacAdam ellipse around any point on the black body locus having a CCT between the CCT of the first color point and the CCT of the second color point. In further implementations, lighting systems can output third white light at third color points along a predetermined path shifted −7±2 DUV from the black body locus having a correlated color temperature between the CCT of the first color point and the CCT of the second color point.

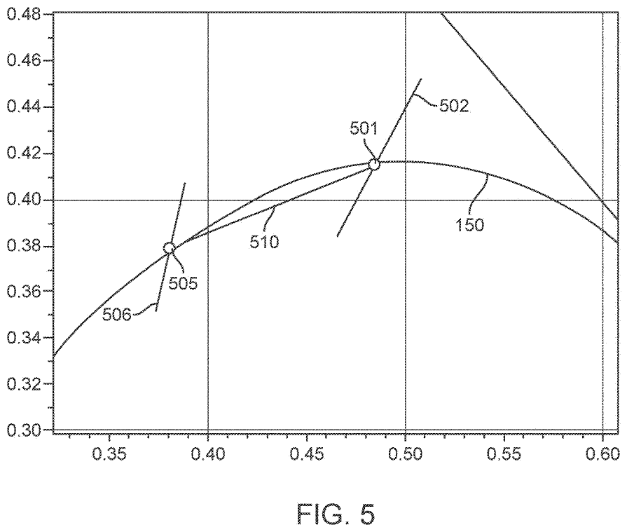

FIG. 5 schematically shows aspects of implementations of lighting systems of the present disclosure. A first color point 505 and a second color points 501 are shown, along with respective constant CCT lines 506 and 502 for reference. As depicted, the first color point 505 has a CCT value of approximately 4000K, but in other implementations may have other CCT values as described elsewhere herein. As depicted, the second color point 501 has a CCT value of approximately 2400K, but in other implementations may have other CCT values as described elsewhere herein. The first and second color points 505/501 are depicted relative to the black body locus 150 in a portion of the 1931 CIE Chromaticity Diagram. In some implementations, at least a portion of the tie line 510 between the first color point 505 and second color point 501 can form a predefined path of a plurality of third color points generated by the lighting system.

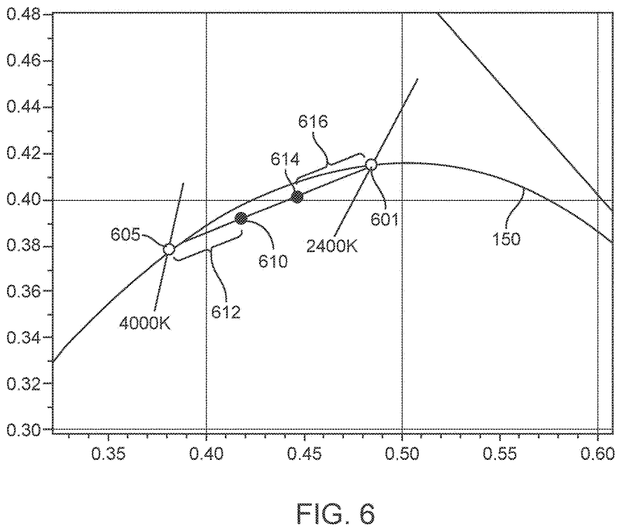

FIG. 6 schematically shows aspects of implementations of lighting systems of the present disclosure. A first color point 605 and a second color point 601 are shown, along with respective constant CCT lines labeled with the approximate respective CCT values 4000K and 2400K of the first and second color points 605/601 for reference. As depicted, the first color point 605 has a CCT value of approximately 4000K, but in other implementations may have other CCT values as described elsewhere herein. As depicted, the second color point 601 has a CCT value of approximately 2400K, but in other implementations may have other CCT values as described elsewhere herein. The first and second color points 605/601 are depicted relative to the black body locus 150 in a portion of the 1931 CIE Chromaticity Diagram. A tie line between the first color point 605 and second color point 601 can form a predefined path of a plurality of third color points generated by the lighting system. In some implementations, a first threshold point 610 can lie on the tie line and have a first threshold CCT value. In further implementations, a second threshold point 614 can lie on the tie line and have a second threshold CCT value. The third color point can have particular characteristics for points in a region 612 on the tie line between the first color point 605 and the first threshold point 610. The third color point can have particular characteristics for points in a region 616 on the tie line between the second color point 601 and the second threshold point 614.

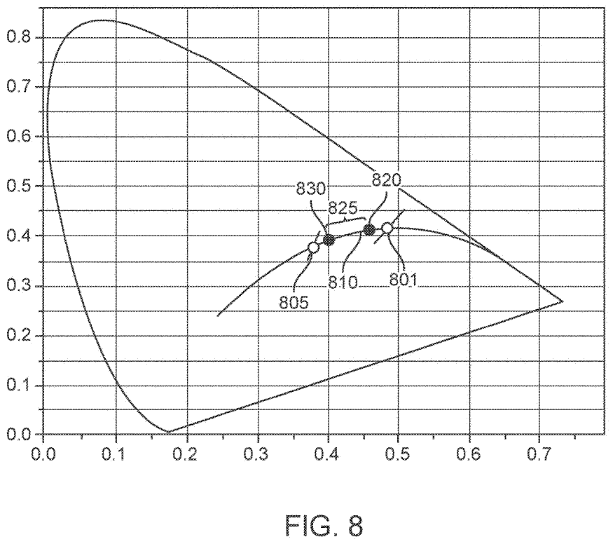

FIG. 8 schematically shows aspects of implementations of lighting systems of the present disclosure. A first color point 805 and a second color point 801 are shown, along with respective constant CCT lines labeled with the approximate respective CCT values 4000K and 2400K of the first and second color points 805/801 for reference. As depicted, the first color point 805 has a CCT value of approximately 4000K, but in other implementations may have other CCT values as described elsewhere herein. As depicted, the second color point 801 has a CCT value of approximately 2400K, but in other implementations may have other CCT values as described elsewhere herein. The first and second color points 805/801 are depicted relative to the black body locus 150 in a portion of the 1931 CIE Chromaticity Diagram. A tie line 810 between the first color point 805 and second color point 801 can form a predefined path of a plurality of third color points generated by the lighting system. In some implementations, a first threshold point 830 can lie on the tie line and have a first threshold CCT value. In further implementations, a second threshold point 820 can lie on the tie line and have a second threshold CCT value. The third color point can have particular characteristics for points in a region 825 on the tie line between the first threshold point 830 and the second threshold point 820. The third color point can have particular characteristics for points in a region on the tie line between the first color point 805 and the first threshold point 830. The third color point can have particular characteristics for points in a region on the tie line between the second color point 801 and the second threshold point 820.

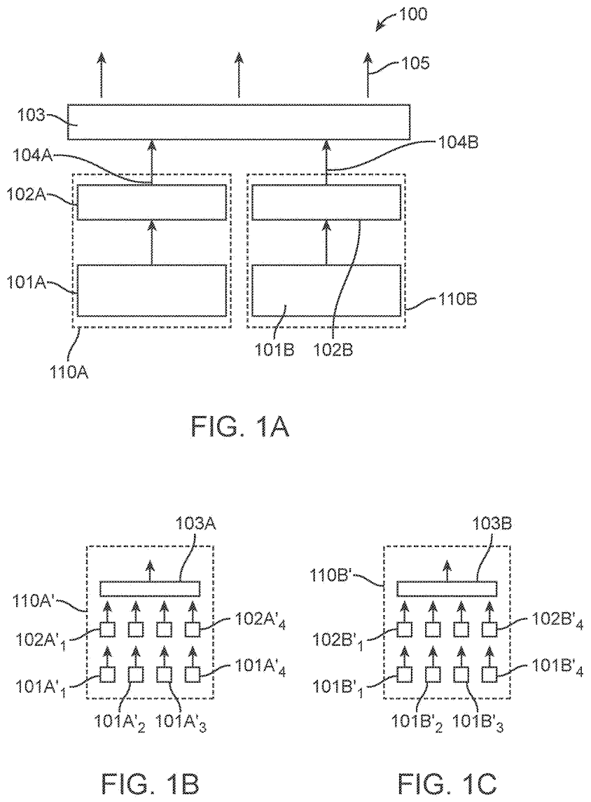

In some implementations, the first and second lighting channels are each provided as one or more LEDs that emit saturated light that excites one or more luminescent materials in a luminophoric medium. FIG. 1 schematically depicts aspects of lighting systems of the present disclosure. An exemplary lighting system 100 is depicted schematically with first and second lighting channels. A first lighting channel 110A having a first LED 101A and a first luminophoric medium 102A together can emit a first white light 104A having a first color point and a first spectral power distribution. The combination of the first LED 101A and the first luminophoric medium 102A together form the first lighting channel. First lighting channels of the present disclosure can comprise a single pair of an LED and a luminophoric medium, as depicted schematically in FIG. 1, or alternatively can comprise a plurality of pairs of LEDs and associated luminophoric mediums in an LED string. FIG. 1B schematically depicts alternative implementations of first lighting channel 110A′ formed from a plurality of LEDs 101A′₁, 101A′₂, 101A′₃, and 101A′₄, that are each associated with a respective luminophoric medium 102A′₁, 102A′₂, 102A′₃, and 102A′₄, which can optionally have the emitted light mixed together by an optional first-lighting-channel optical element 103A. A second lighting channel having a first LED 101B and a second luminophoric medium 102B together can emit a second white light 104B having a second color point and a second spectral power distribution. The combination of the second LED 101B and the second luminophoric medium 102B together form the second lighting channel. Second lighting channels of the present disclosure can comprise a single pair of an LED and a luminophoric medium, as depicted schematically in FIG. 1, or alternatively can comprise a plurality of pairs of LEDs and associated luminophoric mediums in an LED string. FIG. 1C schematically depicts alternative implementations of second lighting channel 110B′ formed from a plurality of LEDs 101B′₁, 101B′₂, 101B′₃, and 101B′₄, that are each associated with a respective luminophoric medium 102B′₁, 102B′₂, 102B′₃, and 102B′₄, which can optionally have the emitted light mixed together by an optional second-lighting-channel optical element 103B. The first white light 104A and the second white light 104B can be combined together to form a third white light 105. The first and second white lights 104A/104B can be passed through one or more optical elements 103 in order to mix, combine, or shape the light output from the lighting system as desired.

The recipient luminophoric mediums 102A, 102B, 102A′₁, 102A′₂, 102A′₃, 102A′₄, 102B′1, 102B′₂, 102B′₃, and 102B′₄ can includes one or more luminescent materials and can be positioned to receive light that is emitted by an LED or other semiconductor light emitting device. In some implementations, recipient luminophoric mediums include layers having luminescent materials that are coated or sprayed directly onto a semiconductor light emitting device or on surfaces of the packaging thereof, and clear encapsulants that include luminescent materials that are arranged to partially or fully cover a semiconductor light emitting device. A recipient luminophoric medium may include one medium layer or the like in which one or more luminescent materials are mixed, multiple stacked layers or mediums, each of which may include one or more of the same or different luminescent materials, and/or multiple spaced apart layers or mediums, each of which may include the same or different luminescent materials. Suitable encapsulants are known by those skilled in the art and have suitable optical, mechanical, chemical, and thermal characteristics. In some implementations, encapsulants can include dimethyl silicone, phenyl silicone, epoxies, acrylics, and polycarbonates. In some implementations, a recipient luminophoric medium can be spatially separated (i.e., remotely located) from an LED or surfaces of the packaging thereof. In some implementations, such spatial segregation may involve separation of a distance of at least about 1 mm, at least about 2 mm, at least about 5 mm, or at least about 10 mm. In certain embodiments, conductive thermal communication between a spatially segregated luminophoric medium and one or more electrically activated emitters is not substantial. Luminescent materials can include phosphors, scintillators, day glow tapes, nanophosphors, inks that glow in visible spectrum upon illumination with light, semiconductor quantum dots, or combinations thereof. In some implementations, the luminescent materials may comprise phosphors comprising one or more of the following materials: BaMg₂Al₁₆O₂₇:Eu²⁺, BaMg₂Al₁₆O₂₇:Eu²⁺,Mn²⁺, CaSiO₃:Pb,Mn, CaWO₄:Pb, MgWO₄, Sr₅Cl(PO₄)₃:Eu²⁺, Sr₂P₂O₇:Sn²⁺, Sr₆P₅BO₂₀:Eu, Ca₅F(PO₄)₃:Sb, (Ba,Ti)₂P₂O₇:Ti, Sr₅F(PO₄)₃:Sb,Mn, (La,Ce,Tb)PO₄:Ce,Tb, (Ca,Zn,Mg)₃(PO₄)₂:Sn, (Sr,Mg)₃(PO₄)₂:Sn, Y₂O₃:Eu³⁺, Mg₄(F)GeO₆:Mn, LaMgAl₁₁O₁₉:Ce, LaPO₄:Ce, SrAl₁₂O₁₉:Ce, BaSi₂O₅:Pb, SrB₄O₇:Eu, Sr₂MgSi₂O₇:Pb, Gd₂O₂S:Tb, Gd₂O₂S:Eu, Gd₂O₂S:Pr, Gd₂O₂S:Pr,Ce,F, Y₂O₂S:Tb, Y₂O₂S:Eu, Y₂O₂S:Pr, Zn(0.5)Cd(0.4)S:Ag, Zn(0.4)Cd(0.6)S:Ag, Y₂SiO₅:Ce, YAlO₃:Ce, Y₃(Al,Ga)₅O₁₂:Ce, CdS:In, ZnO:Ga, ZnO:Zn, (Zn,Cd)S:Cu,Al, ZnCdS:Ag,Cu, ZnS:Ag, ZnS:Cu, NaI:Tl, CsI:Tl, ⁶LiF/ZnS:Ag, ⁶LiF/ZnS:Cu,Al,Au, ZnS:Cu,Al, ZnS:Cu,Au,Al, CaAlSiN₃:Eu, (Sr,Ca)AlSiN₃:Eu, (Ba,Ca,Sr,Mg)₂SiO₄:Eu, Lu₃Al₅O₁₂:Ce, Eu³⁺(Gd_{0.9}Y_{0.1})₃Al₅O₁₂:Bi³⁺,Tb³⁺, Y₃Al₅O₁₂:Ce, (La,Y)₃Si₆N₁₁:Ce, Ca₂AlSi₃O₂N₅:Ce³⁺, Ca₂AlSi₃O₂N₅:Eu²⁺, BaMgAl₁₀O₁₇:Eu, Sr₅(PO₄)₃Cl: Eu, (Ba,Ca,Sr,Mg)₂SiO₄:Eu, Si_{6-z}Al_{z}N_{8-z}Oz:Eu (wherein 0<z≤4.2); M₃Si₆O₁₂N₂:Eu (wherein M=alkaline earth metal element), (Mg,Ca,Sr,Ba)Si₂O₂N₂:Eu, Sr₄Al₁₄O₂₅:Eu, (Ba,Sr,Ca)Al₂O₄:Eu, (Sr,Ba)Al₂Si₂O₈:Eu, (Ba,Mg)₂SiO₄:Eu, (Ba,Sr,Ca)₂(Mg, Zn)Si₂O₇:Eu, (Ba,Ca,Sr,Mg)₉(Sc,Y,Lu,Gd)₂(Si,Ge)₆O₂₄: Eu, Y₂SiO₅:CeTb, Sr₂P₂O₇—Sr₂B₂O₅:Eu, Sr₂Si₃O₈-2SrCl₂:Eu, Zn₂SiO₄:Mn, CeMgAl₁₁O₁₉:Tb, Y₃Al₅O₁₂:Tb, Ca₂Y₈(SiO₄)₆O₂:Tb, La₃Ga₅SiO₁₄:Tb, (Sr,Ba,Ca)Ga₂S₄:Eu,Tb,Sm, Y₃(Al,Ga)₅O₁₂:Ce, (Y,Ga,Tb,La,Sm,Pr,Lu)₃(Al,Ga)₅O₁₂:Ce, Ca₃Sc₂Si₃O₁₂:Ce, Ca₃(Sc,Mg,Na,Li)₂Si₃O₁₂:Ce, CaSc₂O₄:Ce, Eu-activated β-Sialon, SrAl₂O₄:Eu, (La,Gd,Y)₂O₂S:Tb, CeLaPO₄:Tb, ZnS:Cu,Al, ZnS:Cu,Au,Al, (Y,Ga,Lu,Sc,La)BO₃:Ce,Tb, Na₂Gd₂B₂O₇:Ce,Tb, (Ba,Sr)₂(Ca,Mg,Zn)B₂O₆:K,Ce,Tb, Ca₈Mg (SiO₄)₄Cl₂:Eu,Mn, (Sr,Ca,Ba)(Al,Ga,In)₂S₄:Eu, (Ca,Sr)₈(Mg,Zn)(SiO₄)₄Cl₂:Eu,Mn, M₃Si₆O₉N₄:Eu, Sr₅Al₅Si₂₁O₂N₃₅:Eu, Sr₃Si₁₃Al₃N₂₁O₂:Eu, (Mg,Ca,Sr,Ba)₂Si₅N₈:Eu, (La,Y)₂O₂S:Eu, (Y,La,Gd,Lu)₂O₂S:Eu, Y(V,P)O₄:Eu, (Ba,Mg)₂SiO₄:Eu,Mn, (Ba,Sr, Ca,Mg)₂SiO₄:Eu,Mn, LiW₂O₈:Eu, LiW₂O₈:Eu,Sm, Eu₂W₂O₉, Eu₂W₂O₉:Nb and Eu₂W₂O₉:Sm, (Ca,Sr)S:Eu, YAlO₃:Eu, Ca₂Y₈(SiO₄)₆O₂:Eu, LiY₉(SiO₄)₆O₂:Eu, (Y,Gd)₃Al₅O₁₂:Ce, (Tb,Gd)₃Al₅O₁₂:Ce, (Mg,Ca,Sr,Ba)₂Si₅(N,O)₈:Eu, (Mg,Ca,Sr,Ba)Si(N,O)₂:Eu, (Mg,Ca,Sr,Ba)AlSi(N,O)₃:Eu, (Sr,Ca,Ba,Mg)₁₀(PO₄)₆Cl₂:Eu, Mn, Eu,Ba₃MgSi₂O₈:Eu,Mn, (Ba,Sr,Ca,Mg)₃(Zn,Mg)Si₂O₈:Eu,Mn, (k-x)MgO.xAF₂.GeO₂:yMn⁴⁺ (wherein k=2.8 to 5, x=0.1 to 0.7, y=0.005 to 0.015, A=Ca, Sr, Ba, Zn or a mixture thereof), Eu-activated α-Sialon, (Gd,Y,Lu,La)₂O₃:Eu, Bi, (Gd,Y,Lu,La)₂O₂S:Eu,Bi, (Gd,Y, Lu,La)VO₄:Eu,Bi, SrY₂S₄:Eu,Ce, CaLa₂S₄:Ce,Eu, (Ba,Sr,Ca)MgP₂O₇:Eu, Mn, (Sr,Ca,Ba,Mg,Zn)₂P₂O₇:Eu,Mn, (Y,Lu)₂WO₆:Eu,Ma, (Ba,Sr,Ca)ₓSi_{y}N_{z}:Eu,Ce (wherein x, y and z are integers equal to or greater than 1),(Ca,Sr,Ba,Mg)₁₀(PO₄)₆(F,Cl,Br,OH):Eu,Mn, ((Y,Lu,Gd,Tb)_{1-x-y}ScₓCe_{y})₂(Ca,Mg)(Mg,Zn)₂₊ᵣSi_{z-q}Ge_{q}O_{12+δ}, SrAlSi₄N₇, Sr₂Al₂Si₉O₂N₁₄:Eu, M¹ₐM²_{b}M³_{c}O_{d} (wherein M¹=activator element including at least Ce, M²=bivalent metal element, M³=trivalent metal element, 0.0001≤a≤0.2, 0.8≤b≤1.2, 1.6≤c≤2.4 and 3.2≤d≤4.8), A₂₊ₓM_{y}Mn_{z}Fₙ (wherein A=Na and/or K; M=Si and Al, and −1≤x≤1, 0.9≤y+z≤1.1, 0.001≤z≤0.4 and 5≤n≤7), KSF/KSNAF, or (La_{1-x-y}, Euₓ, Ln_{y})₂O₂S (wherein 0.02≤x≤0.50 and 0≤y≤0.50, Ln=Y³⁺, Gd³⁺, Lu³⁺, Sc³⁺, Sm³⁺ or Er³⁺). In some preferred implementations, the luminescent materials may comprise phosphors comprising one or more of the following materials: CaAlSiN₃:Eu, (Sr,Ca)AlSiN₃:Eu, BaMgAl₁₀O₁₇:Eu, (Ba,Ca,Sr,Mg)₂SiO₄:Eu, β-SiAlON, Lu₃Al₅O₁₂:Ce, Eu³⁺(Cd_{0.9}Y_{0.1})₃Al₅O₁₂:Bi³⁺,Tb³⁺, Y₃Al₅O₁₂:Ce, La₃Si₆N₁₁:Ce, (La,Y)₃Si₆N₁₁:Ce, Ca₂AlSi₃O₂N₅:Ce³⁺, Ca₂AlSi₃O₂N₅:Ce³⁺,Eu²⁺, Ca₂AlSi₃O₂N₅:Eu²⁺, BaMgAl₁₀O₁₇:Eu²⁺, Sr_{4.5}Eu_{0.5}(PO₄)₃Cl, or M¹ₐM²_{b}M³_{c}O_{d}. (wherein M¹=activator element comprising Ce, M²=bivalent metal element, M³=trivalent metal element, 0.0001≤a≤0.2, 0.8≤b≤1.2, 1.6≤c≤2.4 and 3.2≤d≤4.8). In further implementations, the luminescent materials may comprise phosphors comprising one or more of the following materials: CaAlSiN₃:Eu, BaMgAl₁₀O₁₇:Eu, Lu₃Al₅O₁₂:Ce, or Y₃Al₅O₁₂:Ce. In yet further implementations, the luminescent materials for the LRNE channels or other channels can comprise phosphors comprising one or more of the following materials excited by light at about 273 nm: LiAlO₂:Fe³⁺ (peak at 770 nm), CdS:Ag⁺,Cl⁻ (peak at 800 nm), ZnSbGaTe:Cr³⁺,Nd³⁺ (peak at 845 nm), La₃In₂Ga₃O₁₂:Cr³⁺, Dy³⁺ (peak at 905 nm), BaGd₂ZnO₅: Yb³⁺ (peak at 979 nm) and Ba(GdY)₂ZnO₅: Yb³⁺ (peak at 979 nm). In further implementations, the luminescent materials can comprise chemically modified versions of these phosphors having excitation bands overlapping with violet or blue LED wavelengths.

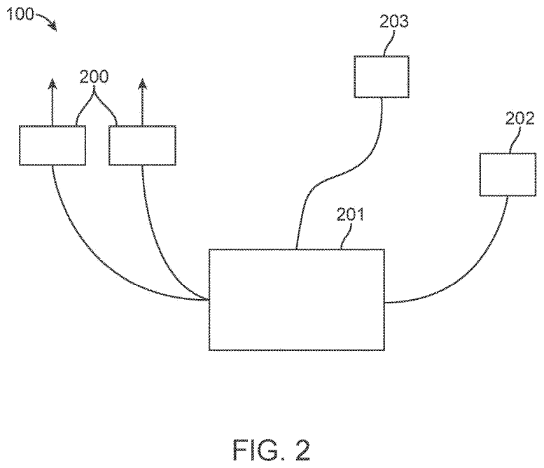

Some implementations of the present invention relate to use of LEDs incorporated into solid state emitter packages. A solid state emitter package typically includes at least one solid state emitter chip that is enclosed with packaging elements to provide environmental and/or mechanical protection, color selection, and light focusing, as well as electrical leads, contacts or traces enabling electrical connection to an external circuit. Encapsulant material, optionally including luminophoric material, may be disposed over solid state emitters in a solid state emitter package. Multiple solid state emitters may be provided in a single package. A package including multiple solid state emitters may include at least one of the following: a single leadframe arranged to conduct power to the solid state emitters, a single reflector arranged to reflect at least a portion of light emanating from each solid state emitter, a single submount supporting each solid state emitter, and a single lens arranged to transmit at least a portion of light emanating from each solid state emitter. Individual LEDs or groups of LEDs in a solid state package (e.g., wired in series) may be separately controlled. As depicted schematically in FIG. 2, multiple solid state emitter packages 200 may be arranged in a single semiconductor light emitting device 100. First and second light channels of the disclosure may be provided in solid state emitter packages 200. Individual solid state emitter packages or groups of solid state emitter packages (e.g., wired in series) may be separately controlled. Separate control of individual emitters, groups of emitters, individual packages, or groups of packages, may be provided by independently applying drive currents to the relevant components with control elements known to those skilled in the art. In one embodiment, at least one control circuit 201 a may include a current supply circuit configured to independently apply an on-state drive current to each individual solid state emitter, group of solid state emitters, individual solid state emitter package, or group of solid state emitter packages. Such control may be responsive to a control signal (optionally including at least one sensor 202 arranged to sense electrical, optical, and/or thermal properties and/or environmental conditions), and a control system 203 may be configured to selectively provide one or more control signals to the at least one current supply circuit. The design and fabrication of semiconductor light emitting devices are well known to those skilled in the art, and hence further description thereof will be omitted. In various embodiments, current to different circuits or circuit portions may be pre-set, user-defined, or responsive to one or more inputs or other control parameters. The lighting systems can be controlled via methods described in U.S. Provisional Patent Application Ser. No. 62/491,137, filed Apr. 27, 2017, entitled Methods and Systems for An Automated Design, Fulfillment, Deployment and Operation Platform for Lighting Installations, U.S. Provisional Patent Application Ser. No. 62/562,714, filed Sep. 25, 2017, entitled Methods and Systems for An Automated Design, Fulfillment, Deployment and Operation Platform for Lighting Installations, and International Patent Application No. PCT/US2018/029380, filed Apr. 25, 2018 and entitled Methods and Systems for an Automated Design, Fulfillment, Deployment and Operation Platform for Lighting Installations, published as International Publication No. WO 2018/200685 A2, each of which hereby are incorporated by reference as if fully set forth herein in their entirety.

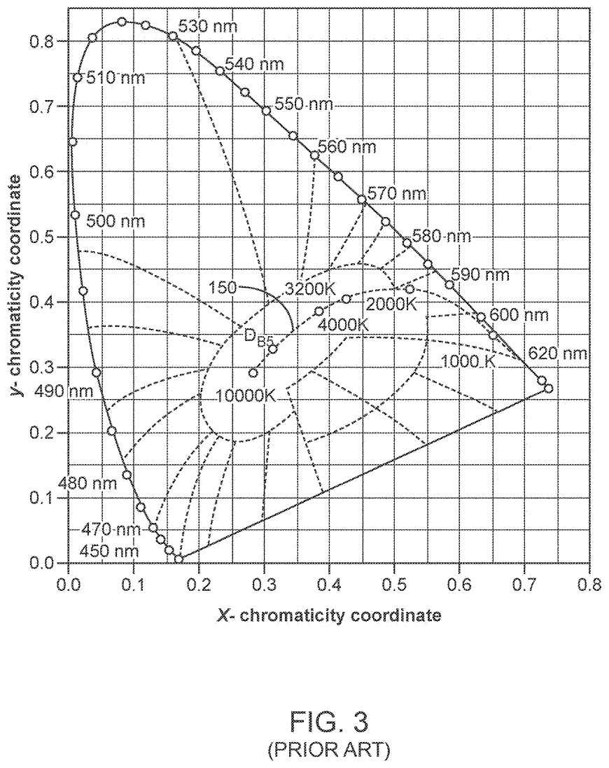

FIG. 3 illustrates a 1931 International Commission on Illumination (CIE) chromaticity diagram. The 1931 CIE Chromaticity diagram is a two-dimensional chromaticity space in which every visible color is represented by a point having x- and y-coordinates, also referred to herein as (ccx, ccy) coordinates. Fully saturated (monochromatic) colors appear on the outer edge of the diagram, while less saturated colors (which represent a combination of wavelengths) appear on the interior of the diagram. The term “saturated”, as used herein, means having a purity of at least 85%, the term “purity” having a well-known meaning to persons skilled in the art, and procedures for calculating purity being well-known to those of skill in the art. The Planckian locus 150, or black body locus (BBL), represented by line 150 on the diagram, follows the color an incandescent black body would take in the chromaticity space as the temperature of the black body changes from about 1000K to 10,000 K. The black body locus goes from deep red at low temperatures (about 1000 K) through orange, yellowish white, white, and finally bluish white at very high temperatures. The temperature of a black body radiator corresponding to a particular color in a chromaticity space is referred to as the “correlated color temperature.” In general, light corresponding to a correlated color temperature (CCT) of about 2700 K to about 6500 K is considered to be “white” light. In particular, as used herein, “white light” generally refers to light having a chromaticity point that is within a 10-step MacAdam ellipse of a point on the black body locus having a CCT between 2700K and 6500K. However, it will be understood that tighter or looser definitions of white light can be used if desired. For example, white light can refer to light having a chromaticity point that is within a seven step MacAdam ellipse of a point on the black body locus having a CCT between 2700K and 6500K. The distance from the black body locus can be measured in the CIE 1960 chromaticity diagram, and is indicated by the symbol Δuv, or DUV or duv as referred to elsewhere herein. If the chromaticity point is above the Planckian locus the DUV is denoted by a positive number; if the chromaticity point is below the locus, DUV is indicated with a negative number. If the DUV is sufficiently positive, the light source may appear greenish or yellowish at the same CCT. If the DUV is sufficiently negative, the light source can appear to be purple or pinkish at the same CCT. Observers may prefer light above or below the Planckian locus for particular CCT values. DUV calculation methods are well known by those of ordinary skill in the art and are more fully described in ANSI C78.377, American National Standard for Electric Lamps—Specifications for the Chromaticity of Solid State Lighting (SSL) Products, which is incorporated by reference herein in its entirety for all purposes. A point representing the CIE Standard Illuminant D65 is also shown on the diagram. The D65 illuminant is intended to represent average daylight and has a CCT of approximately 6500K and the spectral power distribution is described more fully in Joint ISO/CIE Standard, ISO 10526:1999/CIE 5005/E-1998, CIE Standard Illuminants for Colorimetry, which is incorporated by reference herein in its entirety for all purposes.

The color points described in the present disclosure can be within color-point ranges defined by geometric shapes on the 1931 CIE Chromaticity Diagram that enclose a defined set of ccx, ccy color coordinates. It should be understood that any gaps or openings in any described or depicted boundaries for color-point ranges should be closed with straight lines to connect adjacent endpoints in order to define a closed boundary for each color-point range.

The light emitted by a light source may be represented by a point on a chromaticity diagram, such as the 1931 CIE chromaticity diagram, having color coordinates denoted (ccx, ccy) on the X-Y axes of the diagram. A region on a chromaticity diagram may represent light sources having similar chromaticity coordinates.

The ability of a light source to accurately reproduce color in illuminated objects can be characterized using the color rendering index (“CRI”), also referred to as the CIE Ra value. The Ra value of a light source is a modified average of the relative measurements of how the color rendition of an illumination system compares to that of a reference black-body radiator or daylight spectrum when illuminating eight reference colors R1-R8. Thus, the Ra value is a relative measure of the shift in surface color of an object when lit by a particular lamp. The Ra value equals 100 if the color coordinates of a set of test colors being illuminated by the illumination system are the same as the coordinates of the same test colors being irradiated by a reference light source of equivalent CCT. For CCTs less than 5000K, the reference illuminants used in the CRI calculation procedure are the SPDs of blackbody radiators; for CCTs above 5000K, imaginary SPDs calculated from a mathematical model of daylight are used. These reference sources were selected to approximate incandescent lamps and daylight, respectively. Daylight generally has an Ra value of nearly 100, incandescent bulbs have an Ra value of about 95, fluorescent lighting typically has an Ra value of about 70 to 85, while monochromatic light sources have an Ra value of essentially zero. Light sources for general illumination applications with an Ra value of less than 50 are generally considered very poor and are typically only used in applications where economic issues preclude other alternatives. The calculation of CIE Ra values is described more fully in Commission Internationale de l'Éclairage. 1995. Technical Report: Method of Measuring and Specifying Colour Rendering Properties of Light Sources, CIE No. 13.3-1995. Vienna, Austria: Commission Internationale de l'Éclairage, which is incorporated by reference herein in its entirety for all purposes. In addition to the Ra value, a light source can also be evaluated based on a measure of its ability to render a saturated red reference color R9, also known as test color sample 9 (“TCS09”), with the R9 color rendering value (“R9 value”). Light sources can also be evaluated based on a measure of ability to render additional colors R10-R15, which include realistic colors like yellow, green, blue, Caucasian skin color (R13), tree leaf green, and Asian skin color (R15), respectively. Light sources can further be evaluated by calculating the gamut area index (“GAP”). Connecting the rendered color points from the determination of the CIE Ra value in two dimensional space will form a gamut area. Gamut area index is calculated by dividing the gamut area formed by the light source with the gamut area formed by a reference source using the same set of colors that are used for CRI. GAI uses an Equal Energy Spectrum as the reference source rather than a black body radiator. A gamut area index related to a black body radiator (“GAIBB”) can be calculated by using the gamut area formed by the blackbody radiator at the equivalent CCT to the light source.

The ability of a light source to accurately reproduce color in illuminated objects can be characterized using the metrics described in IES Method for Evaluating Light Source Color Rendition, Illuminating Engineering Society, Product ID: TM-30-15 (referred to herein as the “TM-30-15 standard”), which is incorporated by reference herein in its entirety for all purposes. The TM-30-15 standard describes metrics including the Fidelity Index (Rf) and the Gamut Index (Rg) that can be calculated based on the color rendition of a light source for 99 color evaluation samples (“CES”). The 99 CES provide uniform color space coverage, are intended to be spectral sensitivity neutral, and provide color samples that correspond to a variety of real objects. Rf values range from 0 to 100 and indicate the fidelity with which a light source renders colors as compared with a reference illuminant. Rg values provide a measure of the color gamut that the light source provides relative to a reference illuminant. The range of Rg depends upon the Rf value of the light source being tested. The reference illuminant is selected depending on the CCT. For CCT values less than or equal to 4500K, Planckian radiation is used. For CCT values greater than or equal to 5500K, CIE Daylight illuminant is used. Between 4500K and 5500K a proportional mix of Planckian radiation and the CIE Daylight illuminant is used, according to the following equation:

$${S}_{r , M} ( λ , {T}_{t} ) = \frac{5500 - {T}_{t}}{1000} {S}_{r , P} ( λ , {T}_{t} ) + ( 1 - \frac{5500 - {T}_{t}}{1000} ) {S}_{r , D} ( λ , {T}_{t} ) ,$$

where Tₜ is the CCT value, S_{r,M}(λ, Tₜ) is the proportional mix reference illuminant, S_{r,p}(λ, Tₜ) is Planckian radiation, and S_{r,D}(λ, Tₜ) is the CIE Daylight illuminant.

The term “circadian-stimulating energy characteristics” refers to any characteristics of a spectral power distribution that may have biological effects on a subject. In some aspects, the circadian-stimulating energy characteristics of aspects of the lighting systems of this disclosure can include one or more of CS, CLA, EML, BLH, CER, CAF, LEF, circadian power, circadian flux, and the relative amount of power within one or more particular wavelength ranges. Circadian-stimulating energy may be referred to as “CSE”. The application of CSE to biological systems in doses, amount, aliquots and volumes may be referred to as CSE therapy.

Benefits of Blue Light

Exposure to blue light including CSE affects various bio-physiological functions of the human body and may be called “bioactive”. Many of these effects are beneficial. For instance, a region of what is commonly called the blue wavelength region of light may improve memory performance and cognitive function. Exposure to blue wavelength light during memory consolidation has been shown to improve subsequent delayed memory recall when compared to placebo wavelength light exposure. Alkozei, A., Smith R., Dailey N. S., Bajaj S., & Killgore W. D. S. (2017). Acute Exposure to a quantity, volume, aliquot or dose of a specific Blue Wavelength Light During Memory Consolidation Improves Verbal Memory Performance. PLoS ONE 12(9), 1-11. Additionally, blue wavelength light may decrease blood pressure, increase blood flow, and improve overall endothelial function. Full body irradiation with blue light has been shown to promote release nitric oxide from the skin into circulating blood. As a result, systolic blood pressure and vascular resistance have been shown to decrease. Stern, M. et al. (2018). Blue Light Exposure Decreases Systolic Blood Pressure, Arterial Stiffness, and Improves Endothelial Function in Humans. European Journal of Preventive Cardiology 0(00), 1-9.

Challenges of Blue Light.

In some instances exposure to a quantity of blue light may be involved in damage in human eyes. Blue Light Hazard (BLH) is a known risk and the measure of BLH provides a measure of potential for a photochemical induced retinal injury that results from radiation exposure. Such exposure is one factor which has been linked to photoreceptor damage. It has been reported that the blue light appears to decrease Adenosine Triphosphate (ATP) energy production in retinal ganglion cells. This has a negative effect on mitochondrial function and oxidative stress which has been shown to decrease survival of ganglion cells. Tosini, G., Ferguson, I., & Tsubota, K. (2016). Effects of Blue Light on the Circadian System and Eye Physiology. Molecular Vision: Biology and Genetics in Vision Research 22, 61-72. As ganglion cells play a major role in synchronizing circadian rhythms, their destruction inhibits the eye's ability to determine length-of-day and length-of-night. Retinal ganglion cell death further leads to impaired vision. There is also increasing evidence that excessive blue light exposure may cause damage in human skin; it may contribute to wrinkles, worsening skin laxity, and pigmentation issues. Arjmandi, N., Mortazavi G. H., Zarei, S., Faraz M., & Mortazavi, S. A R. (2018). Can Light Emitted from Smartphone Screens and Taking Selfies Cause Premature Aging and Wrinkles? Journal of Biomedical and Physical Engineering 8(4), 447-452. When blue light penetrates the skin it can damage DNA, leading to inflammation, the breakdown of healthy collagen and elastin, and hyperpigmentation. Vandersee, S., Beyer, M., Lademann, J., & Darvin, M. E. (2015). Blue-Violet Light Irradiation Dose Dependently Decreases Carotenoids in Human Skin, Which Indicates the Generation of Free Radicals. Oxidative Medicine and Cellular Longevity. doi: 10.1155/2015/579675. It is also reported that excessive blue light at night negatively affects the human body's natural sleep cycle. Blue light, which inhibits melatonin production, reduces both quantity and quality of sleep. Benefits of Long Red and Near IR. Blue light is not the only light in the visible spectrum that can be used to affect bio-physiological functions (also referred to herein as “bioactive”) of the human body. Recent studies indicate that therapy which may include doses of long red and near-IR: Long Red typically with a spectrum of >625 nms to <700 nms with peak wavelengths >640-670 nm and Near-Infrared typical ranges from >700 nms and <1400 nm (with typical peak wavelengths: 850 nm, 940 nm, 1064 nm) may affect bio-physiological functions and are also described herein as “bioactive” they may improve eye health, skin health, hair growth, and cognitive function. The spectral sensitivity corresponding to the human eye can be considered to be based on the color-matching functions of the 1931 Standard Observer (XYZ tristimulus values for CIE 1931 2° color-matching), which show that the effect of light above 700 nm on color perception to be substantially negligible. In other words, it will have no significant impact on the overall (ccx, ccy) color point on the 1931 CIE Chromaticity Diagram of emitted light from a lighting system. Emissions of Long Red and Near-Infrared may be referred to collectively as Long Red and Near-Infrared Energy (LRNE). How the human eye perceives red, long red and near infrared in a given individual may vary based on a plethora of factors including but not limited to age, stimulation of eye before exposure, eye health and health in general. Accordingly, there will be an overlap between the end of long red and the beginning of near infrared. Those of ordinary skill in the art and the skilled artisan will recognize variation is narrow and does not create substantial uncertainty in the terms. Hence the terminology LRNE is encompasses the entirety of both long red and near-infrared.

Additionally, LRNE may be beneficial by reducing, limiting, counteracting or ameliorating some of the negative effects associated with excessive blue light exposure. Disclosed herein are methods and systems to provide therapeutic doses of LRNE either to address a biological condition or as a prophylactic or health supplement means to limit or prevent at least one of an emotional, neurological, immune, and biological condition or system. “Bioactive Exposure” refers to one or both of LRNE and CSE and directing at least one of LRNE and CSE at a biological system which may be a specific organ or any part of the body.

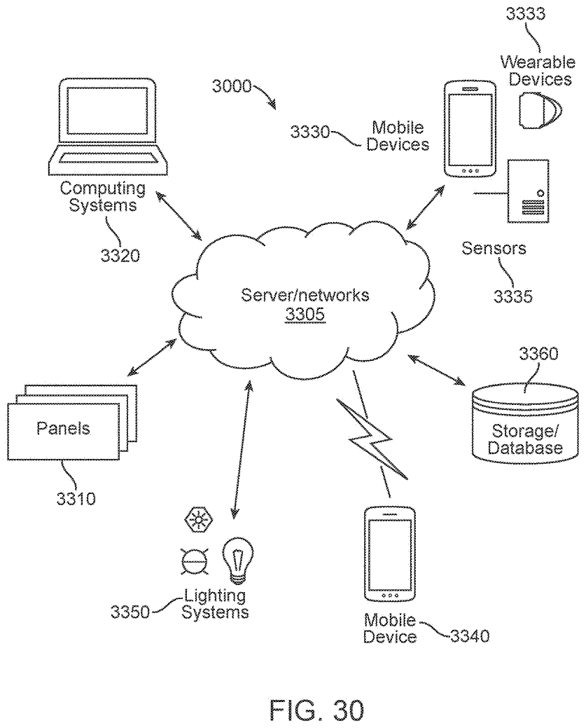

The Bioactive Exposure may be controlled by a control system (described herein, see e.g., FIG. 30 whereby at least one controller, e.g., a computing device receives inputs including fixed, variable and dynamically changing from a variety of sources and the processor associated with the system and method applies at least one of LRNE and CSE in accordance with said control system. Control input data is at least one of input by: users, server, database, derived from a decisioning engine and collected by at least one sensor. The inputs are provided to a processor via signal communication. The processor may be local to the therapeutic device, remote from the therapeutic device or the processing may take place both locally and remote from the therapeutic device. Control systems disclosed herein may adjust the amount and timing of aliquots of bioactive Exposure. The control of aliquots and frequency in response to input may be used to dynamically adjust the therapeutic or health supplement application of one or more of CSE and LRNE to users. Dynamic adjustment of Bioactive Exposure to a user may be viewed as personalized whereby data harvested from sensors in the lighting installation environment as well as sensors that reflect information about users, such as one or more of physiological sensors (including wearable devices, such as armbands, wrist bands, chest bands, glasses, clothing, and the like), sensors on various devices used by a user, ambient sensors, and the like. The control system may have modules within the platform which may connect to or integrate with data sources of information about users as described below. The frequency of the LRNE and CSE are controllable and the range of emission spans constant on to micro-pulses of less than a tenth of a second. Light pulses can be used to provide Bioactive Exposure. Such pulse may have a duration of less than 100 ms, with a frequency between 10 Hz and 0.5 mHz and have been shown to have a bioactive effect, such pulses may vary from a single pulse up to 400,000 pulses.

Disclosed herein are additional methods and systems to provide Bioactive Exposure as one of a supplement and therapeutic dose of LRNE to:

A. Lessen the effect of age-related macular degeneration by stimulating mitochondria in retinal ganglion eye cells to produce more ATP energy. (Calaza, K. C., Kam, J. H., Hogg, C., & Jeffery G. (2015) and Neurobiology of Aging 36, 2869-2876.) The increase in ATP production has been shown to slow the decline in vision associated with aging. LRNE may additionally improve the effects of glaucoma, a condition that destroys ganglion eye cells, by protecting the cornea and the retina. (Olmo-Aguado, S., Núñez-Álvarez, C., & Osborne, N. N. (2016). Red Light of the Visual Spectrum Attenuates Cell Death in Culture and Retinal Ganglion Cell Death in Situ. Acta Ophthalmologica 94, e481-e491).

B. Address a biological condition or as a prophylactic or supplement means to limit or prevent a biological condition. Examples, include but are not limited to, to prevent fluid build-up in the front of the eye, a main complication of glaucoma known to result in cell death of ganglion cells. LRNE has been shown to prevent the death of retinal ganglion cells when the optic nerve has been damaged, thereby preventing vision loss that would otherwise occur. (Kwok-Fai, S., Leung, M. C. P., & Cui, Q. (2014). Effects of Low Laser Treatment on the Survival of Axotomized Retinal Ganglion Cells in Adult Hamsters. Neural Regeneration Research 9(21), 1863-1869.)

C. improve skin health and appearance by the application of LRNE therapy. LRNE can reduce acute and chronic inflammation by increasing blood flow to damaged tissues. (Hamblin, M. R. (2017). Mechanisms and Applications of the Anti-Inflammatory Effects of Photobiomodulation. AIMS Biophysics 4(3), 337-361.) LRNE may be applied to increase natural collagen production, resulting in younger, healthier looking skin. Rats that were exposed to doses of LRN experienced an increase in collagen synthesis and neoformed bone. Brassoliatti, P. et al. (2018). Photobiomodulation on Critical Bone Defects of Rat Calvaria: A Systematic Review. Lasers in Medical Science 33(9), 1841-1848. Patients dealing with acne or depigmentation conditions, such as vitiligo, may benefit from undergoing LRN therapy, as it can control sebum production (which leads to acne), and it can stimulate melanocyte proliferation (which enhances skin re-pigmentation). Skin that has been wounded, burned, or scarred also repairs more rapidly if it is exposed to LRN, as red light significantly increases tensile strength and wound contraction while decreasing inflammation. Avci, P. et al. (2013). Low-level Laser (Light) Therapy (LLLT) in Skin: Stimulating, Healing, Restoring. Semin Cutan Medical Surgery (32)(1), 41-52.

D. A myriad of other bio-physiological function are impacted by LRNEs, including but not limited to, hair growth and cognitive function. LRNE therapy may be used in conjunction with or as an alternative treatment to hormone regulating drugs typically used to treat hair loss. LRNE exposure has been shown to be a treatment in terms of hair regrowth. Gupta, A. K., Mays, et al. (2018). Efficacy of Non-Surgical Treatments for Androgenetic Alopecia: A Systematic Review and Network Meta-Analysis. Journal of The European Academy of Dermatology and Venereology 32(12), 2112-2125. Research has also demonstrated that LRNE exposure may lead to improved cognitive function with few side effects. In one study, those exposed to LRNE experienced quicker reaction times, better memory, a more positive mood, and the ability to learn new information faster. These beneficial effects on the human brain may be related to LRNE's increasing cerebral blood flow and oxygen availability and boost ATP energy production. Hennessy, M., & Hamblin, M. (2017). Photobiomodulation and the Brain: A New Paradigm. Journal of Optics 19(1):013003.

E. LRNE therapy may be able to counteract, limit or ameliorate the negative effects from excessive CSE and blue light exposure. When humans absorb natural blue light from the sun, they also absorb natural red light from the sun—together the two provide numerous health benefits. However, an overload of artificial blue light such as CSE by itself may be determinantal. This damage can be mitigated through LRNE exposure. Balancing and/or controlling a exposure of both artificial blue light and LRNE support wellness benefits similar to those that flow from natural, sunlight exposure.

Circadian illuminance (CLA) is a measure of circadian effective light, spectral irradiance distribution of the light incident at the cornea weighted to reflect the spectral sensitivity of the human circadian system as measured by acute melatonin suppression after a one-hour exposure, and CS, which is the effectiveness of the spectrally weighted irradiance at the cornea from threshold (CS=0.1) to saturation (CS=0.7). The values of CLA are scaled such that an incandescent source at 2856K (known as CIE Illuminant A) which produces 1000 lux (visual lux) will produce 1000 units of circadian lux (CLA). CS values are transformed CLA values and correspond to relative melatonin suppression after one hour of light exposure for a 2.3 mm diameter pupil during the mid-point of melatonin production. CS is calculated from

$$C S = 0.7 ( 1 - \frac{1}{1 + {( \frac{CLA}{355.7} )}^{⋀ 1.126}} ) .$$

The calculation of CLA is more fully described in Rea et al., “Modelling the spectral sensitivity of the human circadian system,” Lighting Research and Technology, 2011; 0: 1-12, and Figueiro et al., “Designing with Circadian Stimulus”, October 2016, LD+A Magazine, Illuminating Engineering Society of North America, which are incorporated by reference herein in its entirety for all purposes. Figueiro et al. describe that exposure to a CS of 0.3 or greater at the eye, for at least one hour in the early part of the day, is effective for stimulating the circadian system and is associated with better sleep and improved behavior and mood.

Equivalent Melanopic Lux (EML) provides a measure of photoreceptive input to circadian and neurophysiological light responses in humans, as described in Lucas et al., “Measuring and using light in the melanopsin age.” Trends in Neurosciences, January 2014, Vol. 37, No. 1, pages 1-9, which is incorporated by reference herein in its entirety, including all appendices, for all purposes. Melanopic lux is weighted to a photopigment with max 480 nm with pre-receptoral filtering based on a 32 year old standard observer, as described more fully in the Appendix A, Supplementary Data to Lucas et al. (2014), User Guide: Irradiance Toolbox (Oxford 18 Oct. 2013), University of Manchester, Lucas Group, which is incorporated by reference herein in its entirety for all purposes. EML values are shown in the tables and Figures herein as the ratio of melanopic lux to luminous flux, with luminous flux considered to be 1000 lumens. It can be desirable for biological effects on users to provide illumination having higher EML in the morning, but lower EML in the late afternoon and evening.

Blue Light Hazard (BLH) provides a measure of potential for a photochemical induced retinal injury that results from radiation exposure. Blue Light Hazard is described in IEC/EN 62471, Photobiological Safety of Lamps and Lamp Systems and Technical Report IEC/TR 62778: Application of IEC 62471 for the assessment of blue light hazard to light sources and luminaires, which are incorporated by reference herein in their entirety for all purposes. A BLH factor can be expressed in (weighted power/lux) in units of μW/cm²/lux.

In some aspects the present disclosure relates to lighting devices and methods to provide light having particular vision energy and circadian energy performance. Many figures of merit are known in the art, some of which are described in Ji Hye Oh, Su Ji Yang and Young Rag Do, “Healthy, natural, efficient and tunable lighting: four-package white LEDs for optimizing the circadian effect, color quality and vision performance,” Light: Science & Applications (2014) 3: e141-e149, which is incorporated herein in its entirety, including supplementary information, for all purposes. Luminous efficacy of radiation (“LER”) can be calculated from the ratio of the luminous flux to the radiant flux (S(λ)), i.e. the spectral power distribution of the light source being evaluated, with the following equation:

$$L E R ( \frac{l m}{W} ) = 683 ( \frac{l m}{W} ) \frac{\int V ( λ ) S ( λ ) d λ}{\int S ( λ ) d λ} .$$

Circadian efficacy of radiation (“CER”) can be calculated from the ratio of circadian luminous flux to the radiant flux, with the following equation:

$$CE R ( \frac{b l m}{W} ) = 683 ( \frac{b l m}{W} ) \frac{\int C ( λ ) S ( λ ) d λ}{\int S ( λ ) d λ} .$$

Circadian action factor (“CAF”) can be defined by the ratio of CER to LER, with the following equation:

$$( \frac{b l m}{l m} ) = \frac{C E R ( \frac{b l m}{W} )}{L E R ( \frac{l m}{W} )} .$$

The term “blm” refers to biolumens, units for measuring circadian flux, also known as circadian lumens. The term “lm” refers to visual lumens. V(λ) is the photopic spectral luminous efficiency function and C(λ) is the circadian spectral sensitivity function. The calculations herein use the circadian spectral sensitivity function, C(λ), from Gall et al., Proceedings of the CIE Symposium 2004 on Light and Health: Non-Visual Effects, 30 Sep.-2 Oct. 2004; Vienna, Austria 2004. CIE: Wien, 2004, pp 129-132, which is incorporated herein in its entirety for all purposes. By integrating the amount of light (milliwatts) within the circadian spectral sensitivity function and dividing such value by the number of photopic lumens, a relative measure of melatonin suppression effects of a particular light source can be obtained. A scaled relative measure denoted as melatonin suppressing milliwatts per hundred lumens may be obtained by dividing the photopic lumens by 100. The term “melatonin suppressing milliwatts per hundred lumens” consistent with the foregoing calculation method is used throughout this application and the accompanying figures and tables. The melatonin suppression index (MSI) of a light source can be calculated from the ratio of the integration of cross product constant lumen spectrum of lamp with melatonin suppression action spectrum in wavelength range 380 nm to 780 nm to the integration of cross product of constant lumen spectrum of Day light spectrum at 6500K with melatonin suppression action spectrum in 380 nm to 780 nm region. The function melatonin suppression action spectrum, “MSAS” or M(λ), is defined by Thapan K, “An action spectrum for melatonin suppression: evidence for a novel non-rod, non-cone photoreceptor system in humans”, Journal of Physiology, 2001, 535: 261-267, which is incorporated herein for all purposes.

The ability of a light source to provide illumination that allows for the clinical observation of cyanosis is based upon the light source's spectral power density in the red portion of the visible spectrum, particularly around 660 nm. The cyanosis observation index (“COI”) is defined by AS/NZS 1680.2.5 Interior Lighting Part 2.5: Hospital and Medical Tasks, Standards Australia, 1997 which is incorporated by reference herein in its entirety, including all appendices, for all purposes. COI is applicable for CCTs from about 3300K to about 5500K, and is preferably of a value less than about 3.3. If a light source's output around 660 nm is too low a patient's skin color may appear darker and may be falsely diagnosed as cyanosed. If a light source's output at 660 nm is too high, it may mask any cyanosis, and it may not be diagnosed when it is present. COI is a dimensionless number and is calculated from the spectral power distribution of the light source. The COI value is calculated by calculating the color difference between blood viewed under the test light source and viewed under the reference lamp (a 4000 K Planckian source) for 50% and 100% oxygen saturation and averaging the results. The lower the value of COI, the smaller the shift in color appearance results under illumination by the source under consideration.

The ability of a light source to accurately reproduce color in illuminated objects can be characterized by the Television Lighting Consistency Index (“TLCI-2012” or “TLCI”) value Qa, as described fully in EBU Tech 3355, Method for the Assessment of the Colorimetric Properties of Luminaires, European Broadcasting Union (“EBU”), Geneva, Switzerland (2014), and EBU Tech 3355-s1, An Introduction to Spectroradiometry, which are incorporated by reference herein in their entirety, including all appendices, for all purposes. The TLCI compares the test light source to a reference luminaire, which is specified to be one whose chromaticity falls on either the Planckian or Daylight locus and having a color temperature which is that of the CCT of the test light source. If the CCT is less than 3400 K, then a Planckian radiator is assumed. If the CCT is greater than 5000 K, then a Daylight radiator is assumed. If the CCT lies between 3400 K and 5000 K, then a mixed illuminant is assumed, being a linear interpolation between Planckian at 3400 K and Daylight at 5000 K. Therefore, it is necessary to calculate spectral power distributions for both Planckian and Daylight radiators. The mathematics for both operations is known in the art and is described more fully in CIE Technical Report 15:2004, Colorimetry 3^{rd} ed., International Commission on Illumination (2004), which is incorporated herein in its entirety for all purposes.

### First Lighting Channels

In some aspects, the present disclosure provides first lighting channels for use in lighting systems. The first lighting channels can have first color points with CCT values between about 4000K and about 6500K. In some implementations, the first color point can have a CCT of about 4000K. In certain implementations, the first color point can have a CCT of about 4000K, about 4100K, about 4200K, about 4300K, about 4400K, about 4500K, about 4600K, about 4700K, about 4800K, about 4900K, about 5000K, about 5100K, about 5200K, about 5300K, about 5400K, about 5500K, about 5600K, about 5700K, about 5800K, about 5900K, about 6000K, about 6100K, about 6200K, about 6300K, about 6400K, or about 6500K.

In some implementations, the first lighting channel can have one or more LEDs having an emission with a first peak wavelength of between about 440 nm and about 510 nm. In certain implementations, the first lighting channel can have one or more LEDs having an emission with a first peak wavelength of about 450 nm.

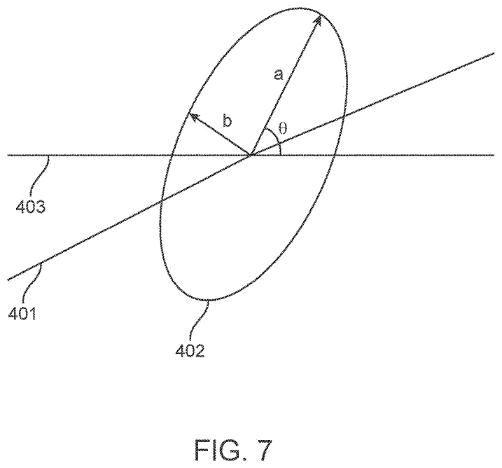

In some implementations, the first lighting channel can have a first color point with a CCT value of about 4000K. The first lighting channel can have a first color point with a color-point range 304A can be defined by a polygonal region on the 1931 CIE Chromaticity Diagram defined by the following ccx, ccy color coordinates: (0.4006, 0.4044), (0.3736, 0.3874), (0.3670, 0.3578), (0.3898, 0.3716), which correlates to an ANSI C78.377-2008 standard 4000K nominal CCT white light with target CCT and tolerance of 3985±275K and target duv and tolerance of 0.001±0.006, as more fully described in American National Standard ANSI C78.377-2008, “Specifications for the Chromaticity of Solid State Lighting Products,” National Electrical Manufacturers Association, American National Standard Lighting Group, which is incorporated herein in its entirety for all purposes. In some implementations, suitable color-point ranges for the first color point can be described as MacAdam ellipse color ranges in the 1931 CIE Chromaticity Diagram color space, as illustrated schematically in FIG. 7, which depicts a color-point range 402, the black body locus 401, and a line 403 of constant ccy coordinates on the 1931 CIE Chromaticity Diagram. In FIG. 7, MacAdam ellipse ranges are described with major axis “a”, minor axis “b”, and ellipse rotation angle θ relative to line 403. In some implementations, the color-point range for the first color point can be range 304B, an embodiment of color range 402, and can be defined as a single 5-step MacAdam ellipse with center point (0.3818, 0.3797) with a major axis “a” of 0.01565, minor axis “b” of 0.00670, with an ellipse rotation angle θ of 52.70°, shown relative to a line 403. In some implementations, the color-point range for the first color point can be range 304C, an embodiment of color range 402, and can be defined as a single 3-step MacAdam ellipse with center point (0.3818, 0.3797) with a major axis “a” of 0.00939, minor axis “b” of 0.00402, with an ellipse rotation angle θ of 53.7°, shown relative to a line 403. In further implementations, the first color point can be within the color-point ranges described in Table 16 for the selected boundary for each nominal CCT value. In other implementations, the color-point range for the first color point can be a region on the 1931 CIE Chromaticity Diagram defined by a polygon connecting the (ccx, ccy) coordinates (0.0.3670, 0.3575), (0.3737, 0.3875), (0.4007, 0.4047), and (0.3898, 0.3720). In yet further implementations, the color-point range for the first color point can be a region on the 1931 CIE Chromaticity Diagram defined by a 4-step MacAdam ellipse centered at 3985K CCT and duv=+0.9845. In other implementations, the color-point range for the first color point can be a region on the 1931 CIE Chromaticity Diagram defined by a polygon connecting the (ccx, ccy) coordinates (0.3703, 0.3590), (0.3851, 0.3679), (0.3942, 0.3972), and (0.3769, 0.3864).

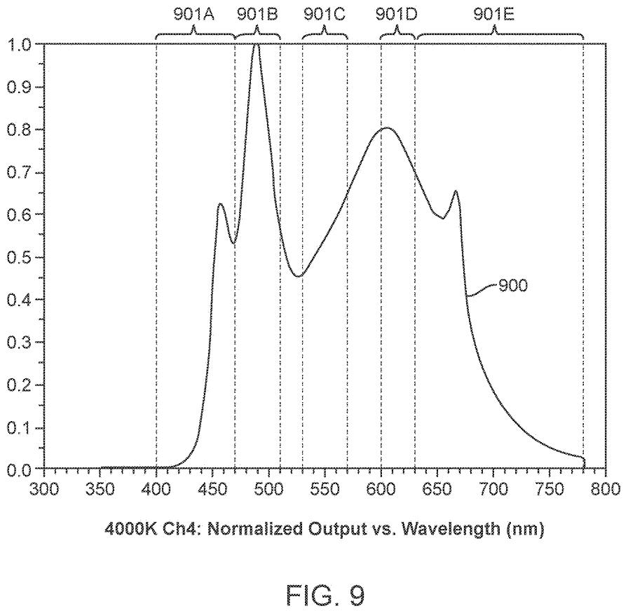

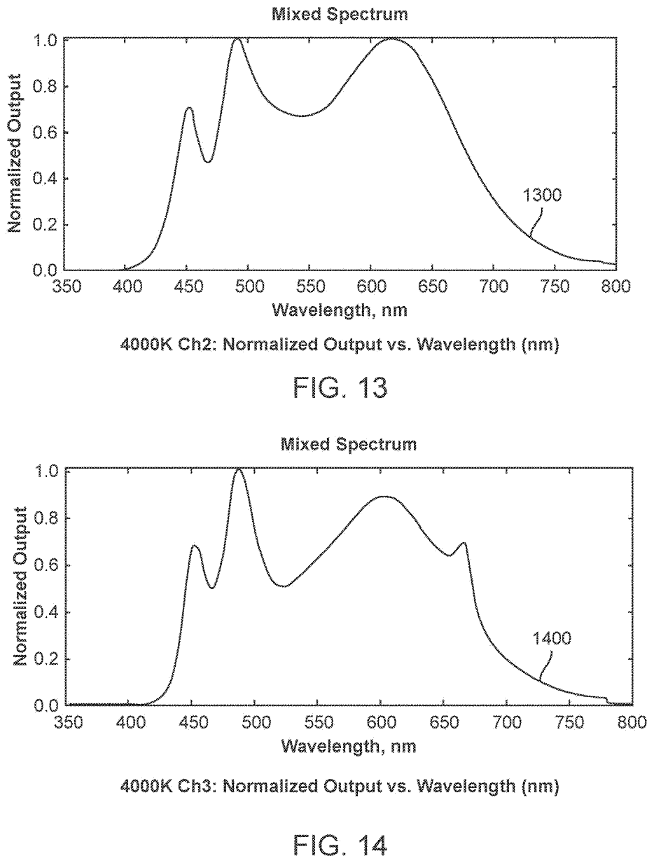

In some implementations, the first lighting channel can have certain spectral power distributions. Some aspects of some exemplary first lighting channels are shown in Table 3. Aspects of the spectral power distributions for the exemplary first lighting channels shown in Table 3 and an average of the exemplary first lighting channels (shown as “Exemplary 1^{st} channels avg”) are provided in Tables 5, 7, 9, 11, and 12, which show the ratios of spectral power within wavelength ranges, with an arbitrary reference wavelength range selected for each exemplary first lighting channel or average thereof and normalized to a value of 100.0, except for Table 12, in which the values are normalized to a value of 1.000. In certain implementations, the first lighting channel can have a first spectral power distribution with spectral power in one or more of the wavelength ranges other than the reference wavelength range increased or decreased within 30% greater or less, within 20% greater or less, within 10% greater or less, or within 5% greater or less than the values shown in Tables 5, 7, 9, 11, and 12. In some implementations, the first lighting channel can have a spectral power distribution that falls between the minimum (shown as “min”) and maximum (shown as “max”) values in each of the wavelength ranges as shown in one or more of the Tables 5, 7, 9, 11, and 12. In further implementations, the first lighting channel can have a spectral power distribution that falls between values 5% less, 10% less, 20% less, or 30% less than the minimum (shown as “min”) and values 5% more, 10% more, 20% more, or 30% more than the maximum (shown as “max”) values in each of the wavelength ranges as shown in one or more of the Tables 5, 7, 9, 11, and 12. FIGS. 9, 13, 14, and 16 depict aspects of first spectral power distributions for the exemplary first lighting channels described herein. FIG. 16 depicts a spectral power distribution 1600 for the exemplary lighting channel “5000K Ch1” listed in Table 3 and further characterized elsewhere herein. FIG. 14 depicts a spectral power distribution 1400 for the exemplary lighting channel “4000K Ch3” listed in Table 3 and further characterized elsewhere herein. FIG. 13 depicts a spectral power distribution 1300 for the exemplary lighting channel “4000K Ch2” listed in Table 3 and further characterized elsewhere herein. FIG. 9 depicts a spectral power distribution 900 for the exemplary lighting channel “4000K Ch4” listed in Table 3 and further characterized elsewhere herein. FIG. 9 further depicts some exemplary wavelength ranges 901A, 901B, 901C, 901D, and 901E, which correspond to the wavelength ranges shown in Table 12. As shown in Table 12, in some implementations, first lighting channels may have particular spectral power values within one or more of wavelength ranges 901A, 901B, 901C, 901D, and 901E, or other wavelength ranges not depicted in FIG. 9 or shown in Table 12 but described elsewhere herein.

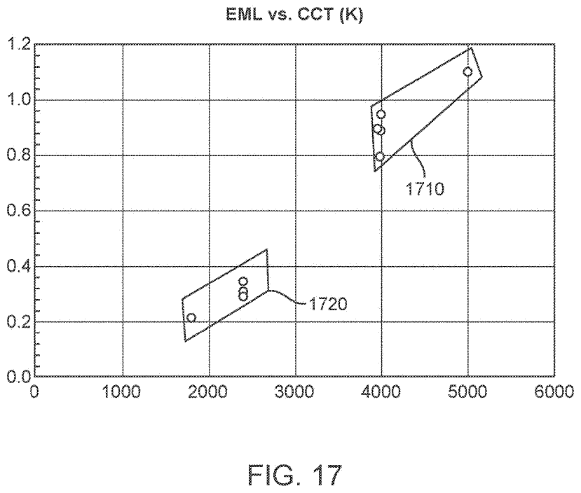

In some aspects, the first lighting channel can have a first white light having a first color point with a CCT and EML value that falls within a range of possible pairings of CCT and EML values, also referred to herein as a CCT-EML range. A suitable CCT-EML range 1710 for first lighting channels of the present disclosure is shown graphically in FIG. 17, which also shows exemplary point pairings of CCT and EML for the exemplary first lighting channels shown in Table 3. Tables 1 and 2 show CCT and EML values for color points generated by some commercially-available fixed-CCT LED-driven white light systems having Ra values of approximately 80.

### Second Lighting Channels

In some aspects, the present disclosure provides second lighting channels for use in lighting systems. The second lighting channels can have second color points with CCT values between about 1800K and about 2700K. In some implementations, the first color point can have a CCT of about 2400K. In some implementations, the first color point can have a CCT of about 1800K, about 1900K, about 2000K, about 2100K, about 2200K, about 2300K, about 2400K, about 2500K, about 2600K, or about 2700K.

In some implementations, the second lighting channel can have one or more LEDs having an emission with a second peak wavelength of between about 380 nm and about 420 nm. In certain implementations, the second lighting channel can have one or more LEDs having an emission with a second peak wavelength of about 410 nm. In some aspects, the use of a different peak wavelength for the LEDs in the second lighting channel in comparison to the LEDs in the first lighting channel can contribute to the desired performance of the lighting systems of the disclosure.

In some implementations of the present disclosure, the second lighting channel can produce light having a second color point within a suitable color-point range. In certain implementations, the second color point can be within the color-point ranges described in Table 16 for the selected boundary for each nominal CCT value. In some implementations, the second color point can be within a color-point range defined by a region bounded by a polygon connecting the (ccx, ccy) coordinates on the 1931 CIE Chromaticity Diagram of (0.4593, 0.3944), (0.5046, 0.4007), (0.5262 0.4381), and (0.4813 0.4319). In further implementations, the second color point can be within a color-point range defined by a region bounded by a 4-step MacAdam ellipse centered at 2370K CCT value and duv=−0.3. In yet further implementations, the second color point can be within a color-point range defined by a region bounded by a polygon connecting the (ccx, ccy) coordinates on the 1931 CIE Chromaticity Diagram of (0.4745, 0.4025), (0.4880, 0.4035), (0.5036, 0.4254), (0.4880, 0.4244).

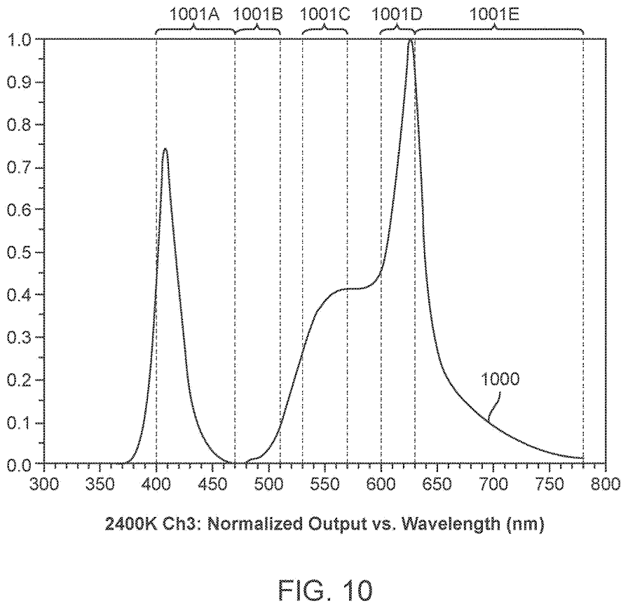

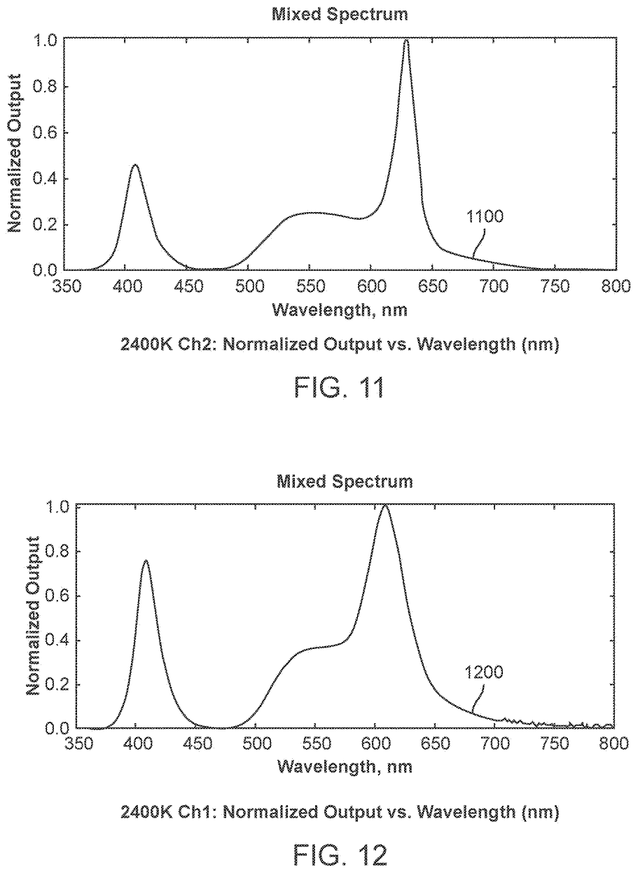

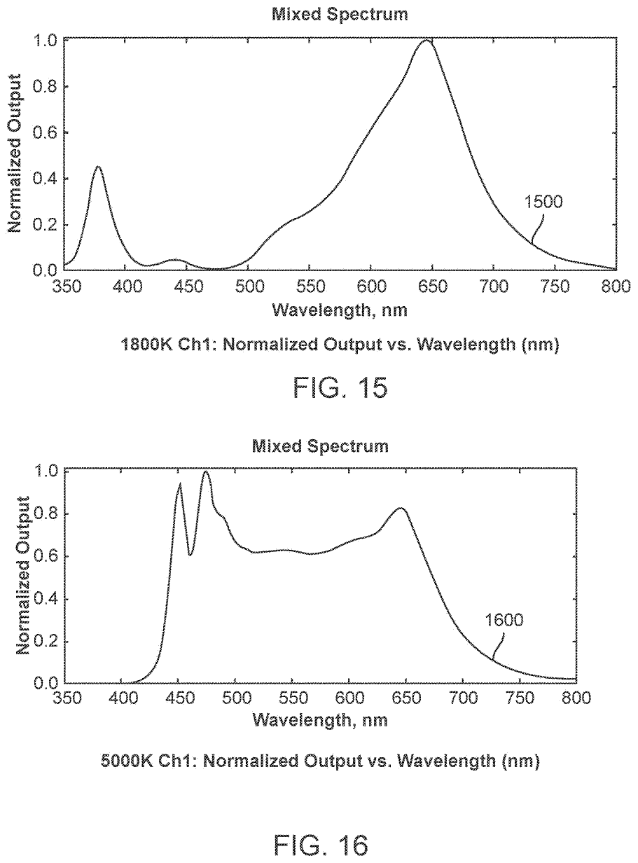

In some implementations, the second lighting channel can have certain spectral power distributions. Some aspects of some exemplary second lighting channels are shown in Table 3. Aspects of the spectral power distributions for the exemplary second lighting channels shown in Table 3 and an average of the exemplary second lighting channels (shown as “Exemplary 2^{nd} channels avg”) are provided in Tables 4, 6, 8, 10, and 12, which show the ratios of spectral power within wavelength ranges, with an arbitrary reference wavelength range selected for each exemplary second lighting channel or average thereof and normalized to a value of 100.0, except for Table 12, in which the values are normalized to a value of 1.000. In certain implementations, the second lighting channel can have a spectral power distribution with spectral power in one or more of the wavelength ranges other than the reference wavelength range increased or decreased within 30% greater or less, within 20% greater or less, within 10% greater or less, or within 5% greater or less than the values shown in one or more of Tables 4, 6, 8, 10, and 12. In some implementations, the second lighting channel can have a spectral power distribution that falls between the minimum (shown as “min”) and maximum (shown as “max”) values in each of the wavelength ranges as shown in one or more of the Tables 4, 6, 8, 10, and 12. In further implementations, the second lighting channel can have a spectral power distribution that falls between values 5% less, 10% less, 20% less, or 30% less than the minimum (shown as “min”) and values 5% more, 10% more, 20% more, or 30% more than the maximum (shown as “max”) values in each of the wavelength ranges as shown in one or more of the Tables 4, 6, 8, 10, and 12. FIG. 11 depicts a spectral power distribution 1100 for the exemplary lighting channel “2400K Ch2” listed in Table 3 and further characterized elsewhere herein. FIG. 12 depicts a spectral power distribution 1200 for the exemplary lighting channel “2400K Ch3” listed in Table 3 and further characterized elsewhere herein. FIG. 15 depicts a spectral power distribution 1500 for the exemplary lighting channel “1800K Ch1” listed in Table 3 and further characterized elsewhere herein. FIG. 10 depicts a spectral power distribution 1000 for the exemplary lighting channel “2400K Ch3” listed in Table 3 and further characterized elsewhere herein. FIG. 10 further depicts some exemplary wavelength ranges 1001A, 1001B, 1001C, 1001D, and 1001E, which correspond to the wavelength ranges shown in Table 12. As shown in Table 12, in some implementations, second lighting channels may have particular spectral power values within one or more of wavelength ranges 1001A, 1001B, 1001C, 1001D, and 1001E, or other wavelength ranges not depicted in FIG. 10 or shown in Table 12 but described elsewhere herein.

In some aspects, the second lighting channel can have a second white light having a second color point with a CCT and EML value that falls within a range of possible pairings of CCT and EML values, also referred to herein as a CCT-EML range. A suitable CCT-EML range 1720 for second lighting channels of the present disclosure is shown graphically in FIG. 17, which also shows exemplary point pairings of CCT and EML for the exemplary second lighting channels shown in Table 3.

### LRNE Lighting Channels

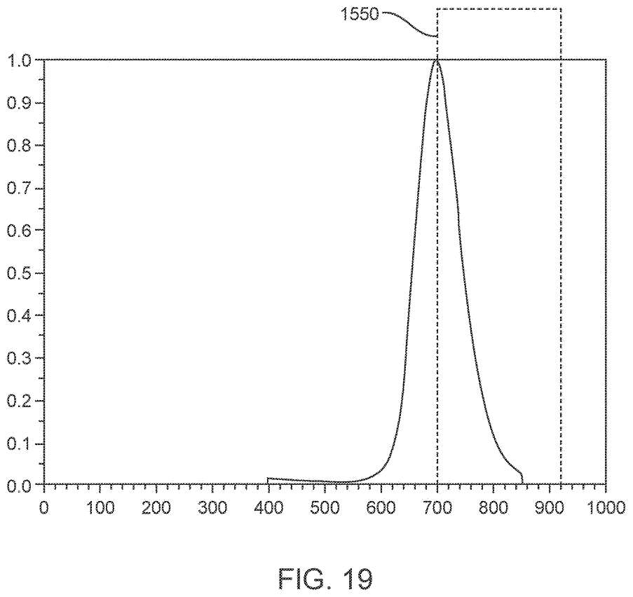

In some aspects, the present disclosure relates to long red and near infrared lighting channels that can provide long red and near infrared energy (“LRNE”). Long red and near infrared channels can provide one or both of Visible LRNE and Non-Visible LRNE. Visible LRNE refers to light having spectral power in wavelengths between about 625 nm and about 700 nm. Non-Visible LRNE refers to light having spectral power in wavelengths greater than or equal to about 700 nm. The Long Red and Near Infrared Channels, also referred to as LRNE channels or LRNE lighting channels, of the present disclosure can be part of one or more red channels involved in color-tuning and providing white light, or as separate channel that can be operated independently of color-tuning or color-rendering requirements. In some implementations an additional LRNE channel includes the non-visible region of the LRNE also referred to as near infrared. Although the near infrared may not be visually perceived as red, such a channel can provide benefits of LRNE as described above. In FIGS. 19-21, Region 1550 represents LRNE emissions which are considered by most to be outside the visual spectrum of humans. FIG. 19 depicts the emission spectrum of an exemplary nitride long-red phosphor, Long-Red Phosphor 700 nm, excited by violet or blue light wavelengths between about 380 nm and about 490 nm. In certain implementations red nitride phosphors having peak wavelengths between about 675 nm and about 775 nm can be included in one or more red channels or long-red channels. Aspects of exemplary phosphors that may be suitable for use in LRNE channels of the present disclosure are shown in Tables B-1, B-2, and B-3.

TABLE B-l

| | 320 < | 400 < | 500 < | 600 < | 700 < | 800 < | 900 < |
|---|---|---|---|---|---|---|---|
| | λ ≤ | λ ≤ | λ ≤ | λ ≤ | λ ≤ | λ ≤ | λ ≤ |
| | 400 | 500 | 600 | 700 | 800 | 900 | 1000 |
| Long-Red | 0.25 | 0.82 | 3.08 | 100.00 | 44.06 | 1.09 | 0.00 |
| Phosphor 675 nm | | | | | | | |
| Long-Red | 0.01 | 1.28 | 1.66 | 100.00 | 110.30 | 5.36 | 0.00 |
| Phosphor 700 nm | | | | | | | |

TABLE B-2

| | 320 < | 380 < | 420 < | 460 < | 500 < | 540 < | 580 < | 620 < |
|---|---|---|---|---|---|---|---|---|
| | λ ≤ | λ ≤ | λ ≤ | λ ≤ | λ ≤ | λ ≤ | λ ≤ | λ ≤ |
| | 380 | 420 | 460 | 500 | 540 | 580 | 620 | 660 |
| Long-Red | 0.2 | 1.0 | 0.9 | 0.8 | 0.8 | 2.3 | 20.2 | 100.0 |
| Phosphor | | | | | | | | |
| 675 nm | | | | | | | | |
| Long-Red | 0.0 | 1.2 | 2.2 | 2.0 | 1.8 | 2.0 | 12.1 | 100.0 |
| Phosphor | | | | | | | | |
| 700 nm | | | | | | | | |

| | 660 < | 700 < | 740 < | 780 < | 820 < | 860 < | 900 < |
|---|---|---|---|---|---|---|---|
| | λ ≤ | λ ≤ | λ ≤ | λ ≤ | λ ≤ | λ ≤ | λ ≤ |
| | 700 | 740 | 780 | 820 | 860 | 900 | 1000 |
| Long-Red | 145.6 | 81.9 | 28.1 | 7.4 | 0.4 | 0.0 | 0.0 |
| Phosphor 675 nm | | | | | | | |
| Long-Red | 314.0 | 304.1 | 134.9 | 41.7 | 8.7 | 0.0 | 0.0 |
| Phosphor 700 nm | | | | | | | |

TABLE B-3

| | 320 < | 340 < | 360 < | 380 < | 400 < | 420 < | 440 < | 460 < | 480 < | 500 < | 520 < | 540 < |
|---|---|---|---|---|---|---|---|---|---|---|---|---|
| | λ ≤ | λ ≤ | λ ≤ | λ ≤ | λ ≤ | λ ≤ | λ ≤ | λ ≤ | λ ≤ | λ ≤ | λ ≤ | λ ≤ |
| | 340 | 360 | 380 | 400 | 420 | 440 | 460 | 480 | 500 | 520 | 540 | 560 |
| Long-Red | 0.0 | 0.0 | 0.2 | 0.8 | 0.7 | 0.7 | 0.7 | 0.6 | 0.6 | 0.6 | 0.6 | 1.0 |
| Phosphor | | | | | | | | | | | | |
| 675 nm | | | | | | | | | | | | |
| Long-Red | 0.0 | 0.0 | 0.0 | 0.1 | 1.6 | 1.6 | 1.5 | 1.4 | 1.4 | 1.3 | 1.3 | 1.3 |
| Phosphor | | | | | | | | | | | | |
| 700 nm | | | | | | | | | | | | |
| | 560 < | 580 < | 600 < | 620 < | 640 < | 660 < | 680 < | 700 < | 720 < | 740 < | 760 < | 780 < |
| | λ ≤ | λ ≤ | λ ≤ | λ ≤ | λ ≤ | λ ≤ | λ ≤ | λ ≤ | λ ≤ | λ ≤ | λ ≤ | λ ≤ |
| | 580 | 600 | 620 | 640 | 660 | 680 | 700 | 720 | 740 | 760 | 780 | 800 |
| Long-Red | 2.7 | 7.9 | 24.2 | 58.6 | 100.0 | 121.2 | 109.6 | 79.7 | 50.2 | 28.9 | 15.7 | 7.9 |
| Phosphor | | | | | | | | | | | | |
| 675 nm | | | | | | | | | | | | |
| Long-Red | 1.6 | 4.3 | 12.5 | 38.2 | 100.0 | 187.6 | 246.3 | 238.7 | 181.5 | 117.4 | 69.0 | 38.2 |
| Phosphor | | | | | | | | | | | | |
| 700 nm | | | | | | | | | | | | |

| | 800 < | 820 < | 840 < | 860 < | 880 < | 900 < | 920 < | 940 < | 960 < | 980 < |
|---|---|---|---|---|---|---|---|---|---|---|
| | λ ≤ | λ ≤ | λ ≤ | λ ≤ | λ ≤ | λ ≤ | λ ≤ | λ ≤ | λ ≤ | λ ≤ |
| | 820 | 840 | 860 | 880 | 900 | 920 | 940 | 960 | 980 | 1000 |
| Long-Red | 3.9 | 0.4 | 0.2 | 0.0 | 0.0 | 0.0 | 0.0 | 0.0 | 0.0 | 0.0 |
| Phosphor | | | | | | | | | | |
| 675 nm | | | | | | | | | | |
| Long-Red | 19.4 | 9.7 | 2.3 | 0.0 | 0.0 | 0.0 | 0.0 | 0.0 | 0.0 | 0.0 |
| Phosphor | | | | | | | | | | |
| 700 nm | | | | | | | | | | |

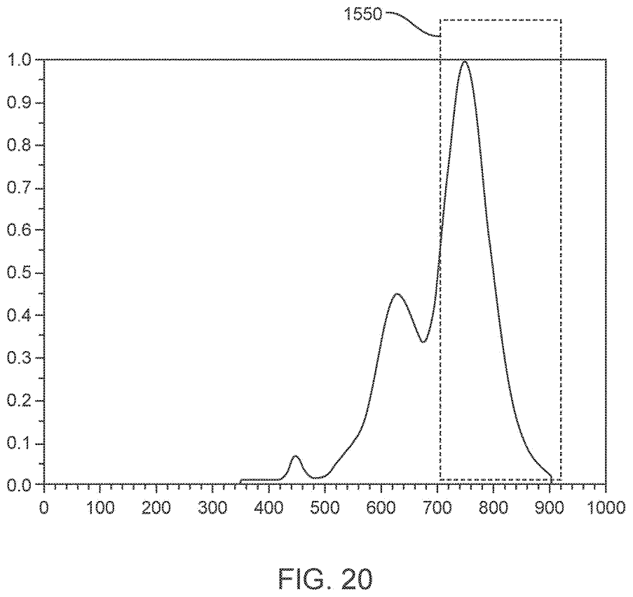

FIG. 20 shows aspects of the spectral power distribution of an exemplary LRNE lighting channel, LRNE Channel B, with further details shown in Tables A-1, A-2, and A-3 below. Three phosphor materials can be provided in a luminophoric medium associated with an LED string in LRNE Channel B, and in some implementations the phosphor materials can include a YAG phosphor, a red phosphor with emission peak at about 630 nm, and a red phosphor with an emission peak at about 750 nm.

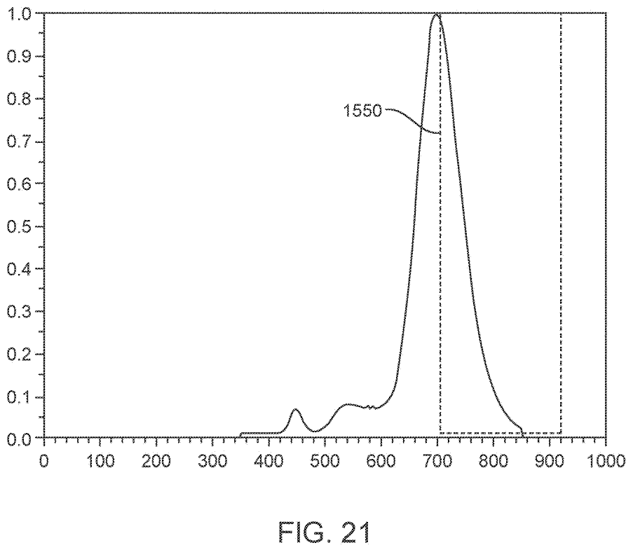

FIG. 21 shows aspects of the spectral power distribution of an exemplary LRNE lighting channel, LRNE Channel A, with further details show in Tables A-1, A-2, and A-3 below. Three phosphor materials can be provided in a luminophoric medium associated with an LED string in LRNE Channel A, and in some implementations the phosphor materials can include a YAG phosphor, a red phosphor with emission peak at about 630 nm, and a red phosphor with an emission peak at about 700 nm.

In some implementations, the LRNE channels can produce red light having certain spectral power distributions. Tables A-1, A-2, and A-3 shows the ratios of spectral power within wavelength ranges, with an arbitrary reference wavelength range selected and normalized to a value of 100.0, for LRNE channels that may be used in some implementations of the disclosure. In some implementations, the LRNE channels can have a spectral power distribution that falls within the ranges between the Exemplary LRNE Channel Minimum and the Exemplary LRNE Channel Maximum in the wavelength ranges shown in Tables A-1, A-2, and A-3. In certain implementations, the LRNE channels of the disclosure can have a spectral power distribution with spectral power in one or more of the wavelength ranges other than the reference wavelength range increased or decreased within 30% greater or less, within 20% greater or less, within 10% greater or less, or within 5% greater or less than the values shown in Tables A-1, A-2, and A-3 for LRNE Channels A-B and the Exemplary LRNE Channel Average.

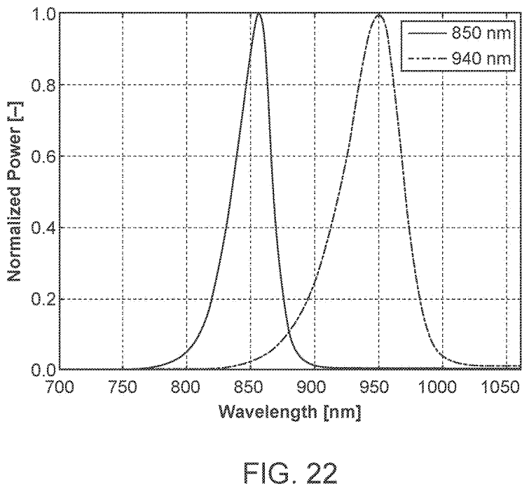

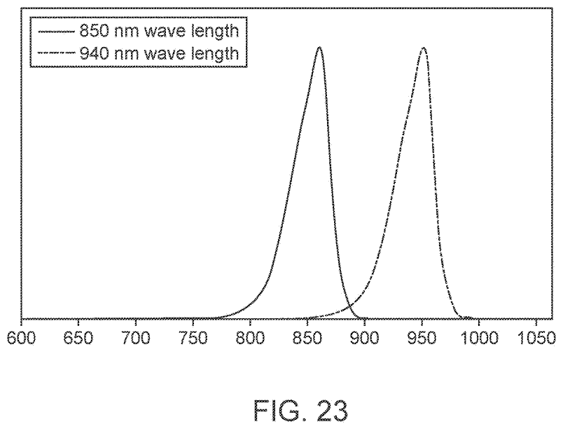

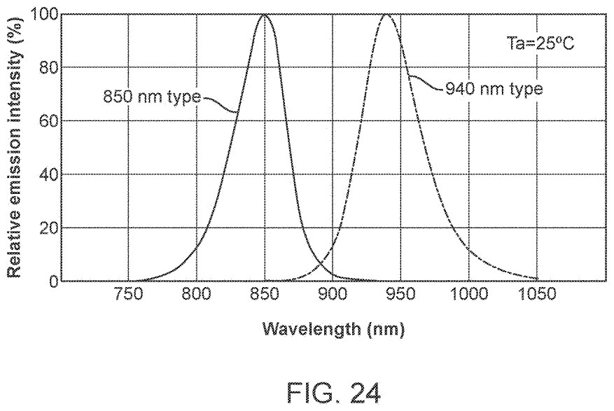

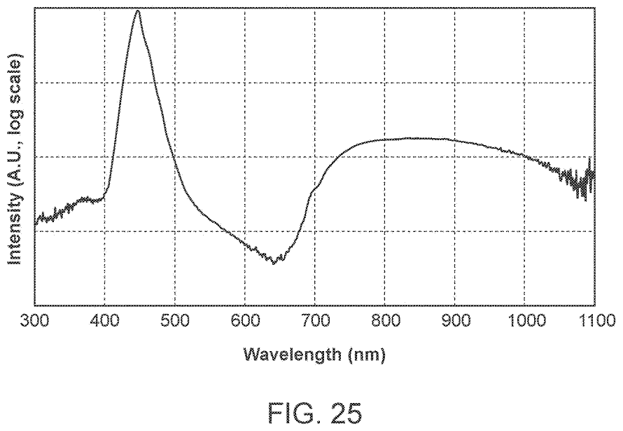

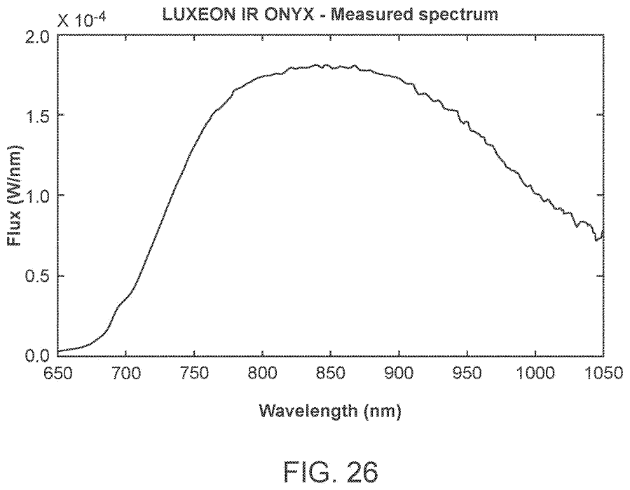

FIGS. 22-26 show aspects of some suitable commercially available LED systems that can be used in the LRNE channels of the present disclosure. FIG. 22 shows the emission spectra of some the LUXEON IR 2720 Line of products from Lumileds (Lumileds Holding B.V., Amsterdam, Netherlands). The peak wavelength of the LUXEON® IR 2720 LED series can be selected from between about 840 nm and about 965 nm as desired for bioactive effects. FIG. 23 shows the emission spectra of some of the SST-10-IR product line from Luminus (Luminus, Inc., Sunnyvale, Calif., USA). Peak wavelengths of 850 nm or 940 nm can be selected. FIG. 24 shows the emission spectra of some 850 nm and 940 nm type LEDs available from Vision Light Tech (Vision Light Tech, Protonenlaan 22, 5405 NE UDEN, The Netherlands). FIG. 25 shows the spectral power characteristics of LUXEON IR ONYX product from Lumileds (Lumileds Holding B.V., Amsterdam, Netherlands). FIG. 26 shows the emission spectra that can be isolated via a Long Pass (LP) filter (such as MidOpt LP920 (Midwest Optical Systems, Inc., 322 Woodwork Lane, Palatine, Ill. 60067 USA)). Between the spectra shown in FIGS. 25 and 26, the LP filter cuts the short wavelength part of the spectrum susceptible to being seen by the human eye. In other implementations, LP filters with higher or lower wavelength thresholds for cut-off can be selected to isolate the spectral power in wavelength ranges as desired for LRNE bioactive effects.

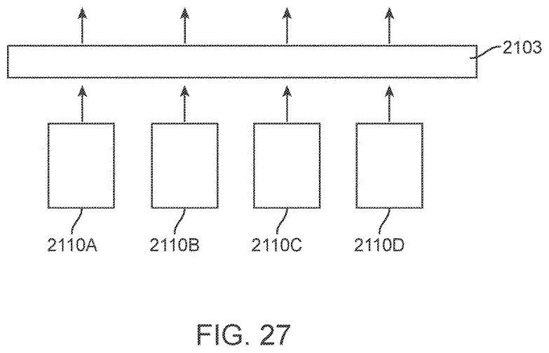

FIG. 27 depicts aspects of variants of the lighting systems shown in FIGS. 1A-1C and 2, with lighting channels 2110A and 2110B correlating to lighting channels 110A/110A′ and 110B/110B′, and additional optional LRNE lighting channels 2110C and 2110D that may be included in the lighting systems in particular implementations. LRNE lighting channels 2110C and 2110D may provide Visible LRNE and Non-Visible LRNE emissions. One or more optical elements 2103, analogous to optical elements 103, can also be provided.

TABLE A-1

| | 320 < | 340 < | 360 < | 380 < | 400 < | 420 < | 440 < | 460 < | 480 < | 500 < | 520 < | 540 < |
|---|---|---|---|---|---|---|---|---|---|---|---|---|
| | λ ≤ | λ ≤ | λ ≤ | λ ≤ | λ ≤ | λ ≤ | λ ≤ | λ ≤ | λ ≤ | λ ≤ | λ ≤ | λ ≤ |
| | 340 | 360 | 380 | 400 | 420 | 440 | 460 | 480 | 500 | 520 | 540 | 560 |
| LRNE Channel A | 0.0 | 1.2 | 1.8 | 1.7 | 2.1 | 6.1 | 13.2 | 4.4 | 3.5 | 10.7 | 17.7 | 18.7 |
| LRNE Channel B | 0.0 | 1.2 | 2.1 | 2.0 | 2.1 | 6.1 | 13.4 | 4.5 | 3.1 | 7.5 | 16.3 | 25.2 |
| Exemplary LRNE | 0.0 | 1.2 | 1.8 | 1.7 | 2.1 | 6.1 | 13.2 | 4.4 | 3.1 | 7.5 | 16.3 | 18.7 |
| Channel Minimum | | | | | | | | | | | | |
| Exemplary LRNE | 0.0 | 1.2 | 1.9 | 1.9 | 2.1 | 6.1 | 13.3 | 4.4 | 3.3 | 9.1 | 17.0 | 21.9 |
| Channel Average | | | | | | | | | | | | |
| Exemplary LRNE | 0.0 | 1.2 | 2.1 | 2.0 | 2.1 | 6.1 | 13.4 | 4.5 | 3.5 | 10.7 | 17.7 | 25.2 |
| Channel Maximum | | | | | | | | | | | | |
| | 560 < | 580 < | 600 < | 620 < | 640 < | 660 < | 680 < | 700 < | 720 < | 740 < | 760 < | 780 < |
| | λ ≤ | λ ≤ | λ ≤ | λ ≤ | λ ≤ | λ ≤ | λ ≤ | λ ≤ | λ ≤ | λ ≤ | λ ≤ | λ ≤ |
| | 580 | 600 | 620 | 640 | 660 | 680 | 700 | 720 | 740 | 760 | 780 | 800 |
| LRNE Channel A | 17.4 | 17.3 | 21.6 | 43.2 | 100.0 | 182.3 | 237.5 | 229.6 | 174.5 | 112.8 | 66.3 | 36.7 |
| LRNE Channel B | 40.3 | 69.5 | 100.8 | 111.3 | 100.0 | 86.9 | 103.1 | 162.2 | 227.8 | 248.3 | 208.2 | 144.4 |
| Exemplary LRNE | 17.4 | 17.3 | 21.6 | 43.2 | 100.0 | 86.9 | 103.1 | 162.2 | 174.5 | 112.8 | 66.3 | 36.7 |
| Channel Minimum | | | | | | | | | | | | |
| Exemplary LRNE | 28.9 | 43.4 | 61.2 | 77.2 | 100.0 | 134.6 | 170.3 | 195.9 | 201.1 | 180.5 | 137.2 | 90.6 |
| Channel Average | | | | | | | | | | | | |
| Exemplary LRNE | 40.3 | 69.5 | 100.8 | 111.3 | 100.0 | 182.3 | 237.5 | 229.6 | 227.8 | 248.3 | 208.2 | 144.4 |
| Channel Maximum | | | | | | | | | | | | |

| | 800 < | 820 < | 840 < | 860 < | 880 < | 900 < | 920 < | 940 < | 960 < | 980 < |
|---|---|---|---|---|---|---|---|---|---|---|
| | λ ≤ | λ ≤ | λ ≤ | λ ≤ | λ ≤ | λ ≤ | λ ≤ | λ ≤ | λ ≤ | λ ≤ |
| | 820 | 840 | 860 | 880 | 900 | 920 | 940 | 960 | 980 | 1000 |
| LRNE Channel A | 18.6 | 9.3 | 2.2 | 0.0 | 0.0 | 0.0 | 0.0 | 0.0 | 0.0 | 0.0 |
| LRNE Channel B | 90.0 | 51.3 | 27.2 | 13.6 | 6.2 | 0.0 | 0.0 | 0.0 | 0.0 | 0.0 |
| Exemplary LRNE | 18.6 | 9.3 | 2.2 | 0.0 | 0.0 | 0.0 | 0.0 | 0.0 | 0.0 | 0.0 |
| Channel Minimum | | | | | | | | | | |
| Exemplary LRNE | 54.3 | 30.3 | 14.7 | 6.8 | 3.1 | 0.0 | 0.0 | 0.0 | 0.0 | 0.0 |
| Channel Average | | | | | | | | | | |
| Exemplary LRNE | 90.0 | 51.3 | 27.2 | 13.6 | 6.2 | 0.0 | 0.0 | 0.0 | 0.0 | 0.0 |
| Channel Maximum | | | | | | | | | | |

TABLE A-2

| | 320 < | 380 < | 420 < | 460 < | 500 < | 540 < | 580 < | 620 < |
|---|---|---|---|---|---|---|---|---|
| | λ ≤ | λ ≤ | λ ≤ | λ ≤ | λ ≤ | λ ≤ | λ ≤ | λ ≤ |
| | 380 | 420 | 460 | 500 | 540 | 580 | 620 | 660 |
| LRNE Channel A | 2.1 | 2.6 | 13.5 | 5.5 | 19.8 | 25.2 | 27.2 | 100.0 |
| LRNE Channel B | 1.6 | 2.0 | 9.2 | 3.6 | 11.3 | 31.0 | 80.6 | 100.0 |
| Exemplary LRNE Channel Minimum | 1.6 | 2.0 | 9.2 | 3.6 | 11.3 | 25.2 | 27.2 | 100.0 |
| Exemplary LRNE Channel Average | 1.8 | 2.3 | 11.4 | 4.5 | 15.5 | 28.1 | 53.9 | 100.0 |
| Exemplary LRNE Channel Maximum | 2.1 | 2.6 | 13.5 | 5.5 | 19.8 | 31.0 | 80.6 | 100.0 |

| | 660 < | 700 < | 740 < | 780 < | 820 < | 860 < | 900 < |
|---|---|---|---|---|---|---|---|
| | λ ≤ | λ ≤ | λ ≤ | λ ≤ | λ ≤ | λ ≤ | λ ≤ |
| | 700 | 740 | 780 | 820 | 860 | 900 | 1000 |
| Long-Red Channel A | 293.2 | 282.2 | 125.1 | 38.7 | 8.1 | 0.0 | 0.0 |
| Long-Red Channel B | 89.9 | 184.6 | 216.1 | 110.9 | 37.2 | 9.3 | 0.0 |
| Exemplary Long-Red Channel Minimum | 89.9 | 184.6 | 125.1 | 38.7 | 8.1 | 0.0 | 0.0 |
| Exemplary Long-Red Channel Average | 191.6 | 233.4 | 170.6 | 74.8 | 22.6 | 4.7 | 0.0 |
| Exemplary Long-Red Channel Maximum | 293.2 | 282.2 | 216.1 | 110.9 | 37.2 | 9.3 | 0.0 |

TABLE A-3

| | 320 < | 400 < | 500 < | 600 < | 700 < | 800 < | 900 < |
|---|---|---|---|---|---|---|---|
| | λ ≤ | λ ≤ | λ ≤ | λ ≤ | λ ≤ | λ ≤ | λ ≤ |
| | 400 | 500 | 600 | 700 | 800 | 900 | 1000 |
| Long-Red Channel A | 0.8 | 5.0 | 14.0 | 100.0 | 106.0 | 5.2 | 0.0 |
| Long-Red Channel B | 1.1 | 5.8 | 31.6 | 100.0 | 197.4 | 37.5 | 0.0 |
| Exemplary Long-Red | 0.8 | 5.0 | 14.0 | 100.0 | 106.0 | 5.2 | 0.0 |
| Channel Minimum | | | | | | | |
| Exemplary Long-Red | 0.9 | 5.4 | 22.8 | 100.0 | 151.7 | 21.3 | 0.0 |
| Channel Average | | | | | | | |
| Exemplary Long-Red | 1.1 | 5.8 | 31.6 | 100.0 | 197.4 | 37.5 | 0.0 |
| Channel Maximum | | | | | | | |

### Circadian-Stimulating Energy Characteristics

In some aspects of the present disclosure, each of the first, second, and third spectral power distributions can have various circadian-stimulating energy characteristics. By selecting appropriate first and second lighting channels, particular circadian-stimulating effects of the lighting systems can be achieved while also providing excellent color-rendering and other lighting performance.

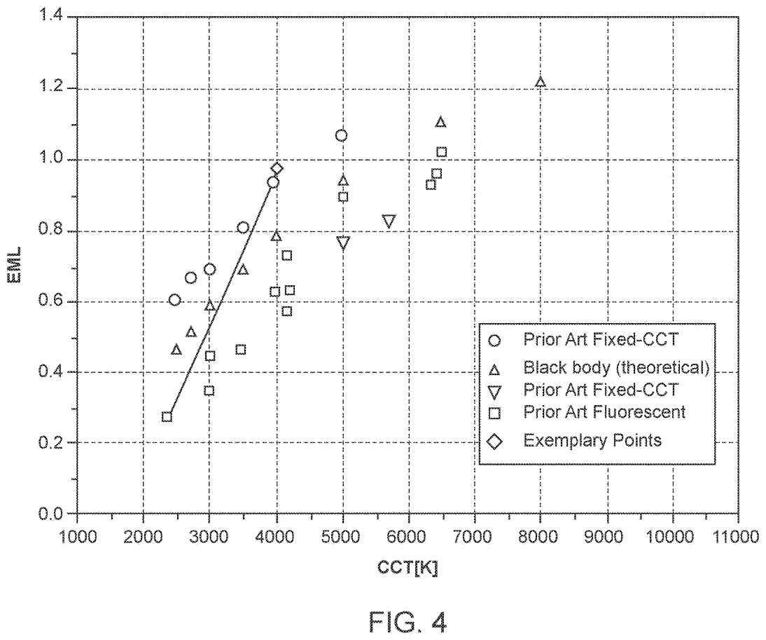

In certain implementations, one or more of the circadian-stimulating energy characteristics of the lighting systems can be EML values of the first, second, and third white light. FIG. 4 graphically shows EML values and CCT values for commercially available LED-based lighting systems having Ra values of approximately 80, fluorescent (“FL”) and incandescent lighting systems, and two exemplary color points for implementations of the present disclosure (diamonds connected by solid black line). In some aspects of the present disclosure, the lighting systems can provide a ratio of a first EML value of the first white light to a second EML value of the second white light. In some implementations, the ratio of the first EML value to the second EML value can be between about 2.0 and about 5.5, between about 3.0 and about 5.0, between about 2.8 and about 3.8, between about 2.6 and about 3.3, between about 4.0 and about 5.5, between about 4.5 and about 5.5, between about 5.5 and about 6.5, between about 6.5 and about 7.5, between about 7.5 and about 8.5, between about 8.5 and about 9.5, between about 2.0 and about 10.0, between about 3.0 and about 10.0, between about 4.0 and about 10.0, between about 5.0 and about 10.0, between about 6.0 and about 10.0, between about 7.0 and about 10.0, between about 8.0 and about 10.0, or between about 9.0 and about 10.0. In further implementations, the ratio of the first EML value to the second EML value can be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6, about 6.1, about 6.2, about 6.3, about 6.4, about 6.5, about 6.6, about 6.7, about 6.8, about 6.9, about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, about 9, about 9.1, about 9.2, about 9.3, about 9.4, about 9.5, about 9.6, about 9.7, about 9.8, about 9.9, or about 10.0. Ratios between the exemplary first and second lighting channels shown in Table 3 are shown in Table 14. In certain implementations, the ratio of the first EML value to the second EML value can be greater than about 2.7 with the first lighting channel producing light with a first color point having a CCT of less than or equal to about 5000K. This can be advantageous as it can provide for a desirable change in EML values while avoiding the use of cool white light, which can be undesirable for certain applications and can have higher BLH factors.

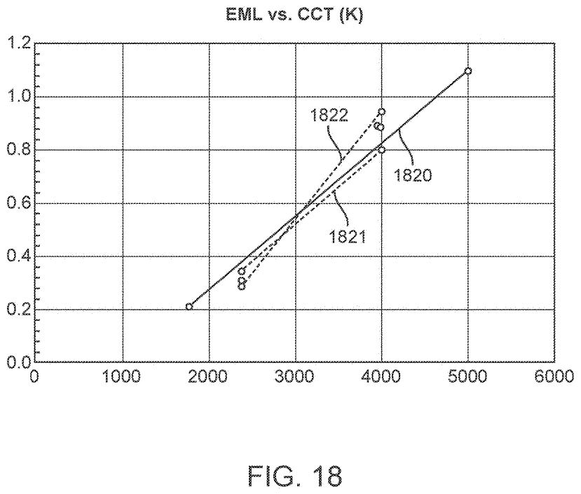

In further aspects of the present disclosure, the lighting systems can provide an EML slope against CCT difference for the first lighting channels and the second lighting channels, also referred to herein as “EML slope.” EML slope against CCT difference between pairings of the exemplary first and second lighting channels shown in Table 3 are shown in Table 13, with the slope values shown per 1000K for ease of reading. Some exemplary EML slope lines 1820, 1821, and 1822 are shown graphically in FIG. 18 for pairings for some pairings of the exemplary first lighting channels and second lighting channels listed in Table 3. In some implementations, the EML slope can be between about 0.25 and about 0.45, about 0.30 and about 0.42, about 0.35 and about 0.42, about 0.38 and about 0.42, between about 0.40 and about 0.42, or between about 0.33 and about 0.40. In further implementations, the EML slope can be about 0.25, about 0.26, about 0.27, about 0.28, about 0.29, about 0.30, about 0.31, about 0.32, about 0.33, about 0.34, about 0.35, about 0.36, about 0.37, about 0.38, about 0.39, about 0.40, about 0.41, about 0.42, about 0.43, about 0.44, or about 0.45.

In further aspects of the present disclosure, lighting systems can have first and second lighting channels with first and second circadian-stimulating energy characteristics that relate to spectral energy within particular wavelength ranges. In some implementations, spectral energy concentrations within particular wavelength ranges can lead to biological effects by providing photostimulation to intrinsically photosensitive retinal ganglion cells (ipRGCs), which express melanopsin, a photopigment that can respond to light directly, and can be associated with non-image-forming functions such as circadian photoentrainment and pupil-size control in addition to some image-forming functions. ipRGCs are sensitive to light at wavelengths between about 400 nm and about 600 nm, with a peak sensitivity and response to light with wavelengths around 480 nm to 490 nm. In certain implementations, the first circadian-stimulating energy characteristic and the second circadian-stimulating energy characteristic can be the percentage of the spectral power in the first spectral power distribution and the second spectral power distribution, respectively, between a first wavelength value and a second wavelength value, forming a particular wavelength range therein greater than the first wavelength value and less than or equal to the second wavelength value. In some implementations, the first wavelength value can be about 400 nm, about 410 nm, about 420 nm, about 430 nm, about 440 nm, about 450 nm, about 460 nm, about 470 nm, about 480 nm, about 490 nm, about 500 nm, about 510 nm, about 520 nm, about 530 nm, about 540 nm, about 550, about 560 nm, about 570 nm, about 580 nm, about 590 nm, or about 600 nm. In some implementations, the second wavelength value can be about 410 nm, about 420 nm, about 430 nm, about 440 nm, about 450 nm, about 460 nm, about 470 nm, about 480 nm, about 490 nm, about 500 nm, about 510 nm, about 520 nm, about 530 nm, about 540 nm, about 550, about 560 nm, about 570 nm, about 580 nm, about 590 nm, about 600 nm, or about 610 nm. In certain implementations, the first wavelength value can be 440 nm and the second wavelength value can be 490 nm, with the particular wavelength range being 440<λ≤490 nm, as shown for values for the exemplary first and second lighting channels shown in Table 3, which shows the percent spectral energy in the range 440<λ≤490 nm in comparison to the total spectral energy in the range 380<λ≤780 nm. In further implementations, other first and second wavelength values can be selected for the first circadian-stimulating energy characteristic and the second circadian-stimulating energy characteristic of the percentages of the spectral power in the first spectral power distribution and the second spectral power distribution between the first and second wavelength values, including but not limited to wavelength ranges (in nm) from about 400 to about 410, about 410 to about 420, about 420 to about 430, about 430 to about 440, about 440 to about 450, about 450 to about 460, about 460 to about 470, about 470 to about 480, about 480 to about 490, about 490 to about 500, about 500 to about 510, about 510 to about 520, about 520 to about 530, about 530 to about 540, about 540 to about 550, or about 550 to about 560. The percentages of the spectral power in the first spectral power distribution and the second spectral power distribution for a particular wavelength range can be obtained or calculated from the data for the exemplary first and second lighting channels shown in Tables 3-12 and the characteristics of suitable first and second lighting channels as described elsewhere herein. Table 15 shows some values for 10-nm wide wavelength ranges between 400 nm and 520 nm, shown as a percentage of spectral energy in the wavelength range in comparison to the total spectral energy from 320 nm to 800 nm. In some implementations, the first circadian-stimulating energy characteristic can be the percentage of spectral energy in one or more of the wavelength ranges shown in Table 15 for the exemplary first lighting channels of Table 3 or the average thereof (“Exemplary 1^{st} channels avg”). In further implementations, the first circadian-stimulating energy characteristic can be between values equal to, 5% less than, 10% less than, 20% less than, or 30% less than the minimum (shown as “Exemplary 1^{st} channels min”) and values equal to, 5% more than, 10% more than, 20% more than, or 30% more than the maximum (shown as “Exemplary 1^{st} channels max”) values in one or more of the wavelength ranges as shown in Table 15. In further implementations, the second circadian-stimulating energy characteristic can be the percentage of the spectral energy in one or more of the wavelength ranges shown in Table 15 for the exemplary second lighting channels of Table 3 or the average thereof (“Exemplary 2^{nd} channels avg”). In further implementations, the second circadian-stimulating energy characteristic can be between values equal to, 5% less than, 10% less than, 20% less than, or 30% less than the minimum (shown as “Exemplary 2^{nd} channels min”) and values equal to, 5% more than, 10% more than, 20% more than, or 30% more than the maximum (shown as “Exemplary 2^{nd} channels max”) values in one or more of the wavelength ranges as shown in Table 15.

In certain implementations, the first circadian-stimulating energy characteristic and the second circadian-stimulating energy characteristic can be the percentage of the spectral power in the first spectral power distribution and the second spectral power distribution, respectively, between a first wavelength value and a second wavelength value, forming a particular wavelength range therein greater than the first wavelength value and less than or equal to the second wavelength value. In some instances, the first and second circadian-stimulating energy characteristics can be one or more of the percentage of spectral power in the wavelength ranges of 470 nm<λ≤480 nm, 480 nm<λ≤490 nm, and 490 nm<λ≤500 nm in comparison to the total energy from 320 nm<λ≤800 nm in the first and second spectral power distributions respectively. In some implementations, for the first lighting channel the percentage of spectral power in the wavelength ranges of 470 nm<λ≤480 nm in comparison to the total energy from 320 nm<λ≤800 nm can be between about 2.50 and about 6.00, between about 3.00 and about 5.50, between about 3.00 and about 4.00, between about 3.50 and about 4.00, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, or about 6.0. In certain implementations, for the first lighting channel the percentage of spectral power in the wavelength ranges of 480 nm<λ≤490 nm in comparison to the total energy from 320 nm<λ≤800 nm can be between about 4.0 and about 6.5, between about 4.5 and about 5.5, between about 4.4 and about 4.6, between about 5.2 and about 5.8, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, or about 6.5. In some implementations, for the first lighting channel the percentage of spectral power in the wavelength ranges of 490 nm<λ≤500 nm in comparison to the total energy from 320 nm<λ≤800 nm can be between about 3.5 and about 6.0, between about 4.0 and about 5.0, between about 4.5 and about 5.5, between about 4.5 and about 5.0, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, or about 6.0. In some implementations, for the second lighting channel the percentage of spectral power in the wavelength ranges of 470 nm<λ≤480 nm in comparison to the total energy from 320 nm<λ≤800 nm can be between about 0.025 and about 0.080, between about 0.030 and about 0.060, between about 0.050 and about 0.070, between about 0.050 and about 0.060, about 0.025, about 0.030, about 0.035, about 0.040, about 0.045, about 0.050, about 0.055, about 0.56, about 0.57, about 0.58, about 0.59, about 0.060, about 0.61, about 0.62, about 0.63, about 0.64, about 0.065, about 0.66, about 0.67, about 0.68, about 0.69, about 0.070, about 0.075, or about 0.080. In certain implementations, for the second lighting channel the percentage of spectral power in the wavelength ranges of 480 nm<λ≤490 nm in comparison to the total energy from 320 nm<λ≤800 nm can be between about 0.10 and about 0.30, between about 0.10 and about 0.15, between about 0.20 and about 0.25, between about 0.13 and about 0.24, about 0.10, about 0.11, about 0.12, about 0.13, about 0.14, about 0.15, about 0.016, about 0.17, about 0.18, about 0.19, about 0.20, about 0.21, about 0.22, about 0.23, about 0.24, about 0.25, about 0.26, about 0.27, about 0.28, about 0.29, or about 0.30. In some implementations, for the second lighting channel the percentage of spectral power in the wavelength ranges of 490 nm<λ≤500 nm in comparison to the total energy from 320 nm<λ≤800 nm can be between about 0.25 and about 0.75, between about 0.25 and about 0.40, between about 0.55 and about 0.70, between about 0.30 and about 0.35, about 0.25, about 0.26, about 0.27, about 0.28, about 0.29, about 0.30, about 0.31, about 0.32, about 0.33, about 0.34, about 0.35, about 0.36, about 0.37, about 0.38, about 0.39, about 0.40, about 0.41, about 0.42, about 0.43, about 0.44, about 0.45, about 0.46, about 0.47, about 0.48, about 0.49, about 0.50, about 0.51, about 0.52, about 0.53, about 0.54, about 0.55, about 0.56, about 0.57, about 0.58, about 0.59, about 0.60, about 0.61, about 0.62, about 0.63, about 0.64, about 0.65, about 0.66, about 0.67, about 0.68, about 0.69, about 0.70, about 0.71, about 0.72, about 0.73, about 0.74, or about 0.75.

In certain implementations, the first spectral power distribution of the first white light produced by the first lighting channel has a first circadian-stimulating energy characteristic, and the second spectral power distribution of the second white light produced by the second lighting channel has a second circadian-stimulating energy characteristic. In some implementations, the first circadian-stimulating energy characteristic can be a first percentage, the first percentage comprising the percentage of the spectral power between 380 nm and 780 nm in the first spectral power distribution between 440 nm and 490 nm. In certain implementations, the second circadian-stimulating energy characteristic can be a second percentage, the second percentage comprising the percentage of the spectral power between 380 nm and 780 nm in the second spectral power distribution between 440 nm and 490 nm. Table 3 shows some exemplary values for the first and second percentages for exemplary first and second lighting channels. In certain implementations of the lighting systems of the present disclosure, the first percentage can be between about 15% and about 25%, between about 16% and about 22%, about 16%, about 17%, about 18%, about 19%, about 20%, about 21%, about 22%, about 23%, about 24%, or about 25%. In further implementations of the lighting systems of the present disclosure, the second percentage can be between about 0.9% and about 1.05%, between about 0.85% and about 0.95%, between about 0.85% and about 0.90%, between about 0.90% and about 0.95%, about 0.90%, about 0.91%, about 0.92%, about 0.93%, about 0.94%, about 0.95%, about 0.96%, about 0.97%, about 0.98%, about 0.99%, about 1.00%, about 1.01%, about 1.02%, about 1.03%, about 1.04%, or about 1.05%. In some implementations, the lighting systems can have a ratio of the first percentage to the second percentage of between about 13 and about 30, between about 15 and about 25, between about 20 and about 25, between about 20 and about 30, between about 18 and about 22, about 13, about 14, about 15, about 16, about 17, about 18, about 19, about 20, about 21, about 22, about 23, about 24, about 25, about 26, about 27, about 28, about 29, or about 30.

In certain aspects, the present disclosure provides lighting systems that can provide the third white light at a plurality of third color points along a predefined path near the black body locus on the 1931 CIE Chromaticity Diagram, with the third color points having particular circadian-stimulating energy characteristics. The third color points can have particular circadian-stimulating energy characteristics at CCT values above or below one or more of a first threshold CCT, a second threshold CCT, or a third threshold CCT or at CCT values between pairs of the first, second, and third threshold CCT values. The second threshold CCT can be about 1800K, about 1900K, about 2000K, about 2100K, about 2200K, about 2300K, about 2400K, about 2500K, about 2600K, about 2700K, about 2800K, about 2900K, about 3000K, about 3100K, or about 3200K. The first threshold CCT can be about 3300K, about 3400K, about 3500K, about 3600K, about 3700K, about 3800K, about 3900K, about 4000K, about 4500K, about 5000K, about 5500K, about 6000K, or about 6500K.

In some implementations, the third color points can have EML values greater than a first EML threshold at CCT values greater than the first threshold CCT and the third color points can have EML values less than a second EML threshold at CCT values less than the second threshold CCT. In certain implementations, the first threshold EML value can be about 0.60 and the first threshold CCT can be about 3300K. In certain implementations, the first threshold EML value can be about 0.60 and the first threshold CCT can be about 3300K. In some implementations, the first threshold EML value can be about 0.75 and the first threshold CCT can be about 3500K. In further implementations, the first threshold EML value can be about 0.85 and the first threshold CCT can be about 3800K. In certain implementations, the second threshold EML value can be about 0.58 and the second threshold CCT can be about 3100K. In certain implementations, the second threshold EML value can be about 0.50 and the second threshold CCT can be about 2900K. In certain implementations, the second threshold EML value can be about 0.43 and the second threshold CCT can be about 2700K. In certain implementations, the second threshold EML value can be about 0.40 and the second threshold CCT can be about 2600K.

### Luminescent Materials

Blends of luminescent materials can be used in luminophoric mediums (102A/102B/102A′₁/102B′₁/102A′₂/102B′₂/102A′₃/102B′₃/102A′₄/102B′₄/102A′ₙ/102B′ₙ) to create luminophoric mediums having the desired saturated color points when excited by their respective LED strings (102A/102B/102A′₁/102B′₁/102A′₂/102B′₂/102A′₃/102B′₃/102A′₄/102B′₄/102A′ₙ/102B′ₙ) including luminescent materials such as those disclosed in co-pending application PCT/US2016/015318 filed Jan. 28, 2016, entitled “Compositions for LED Light Conversions”, the entirety of which is hereby incorporated by this reference as if fully set forth herein. Traditionally, a desired combined output light can be generated along a tie line between the LED string output light color point and the saturated color point of the associated recipient luminophoric medium by utilizing different ratios of total luminescent material to the encapsulant material in which it is incorporated. Increasing the amount of luminescent material in the optical path will shift the output light color point towards the saturated color point of the luminophoric medium. In some instances, the desired saturated color point of a recipient luminophoric medium can be achieved by blending two or more luminescent materials in a ratio. The appropriate ratio to achieve the desired saturated color point can be determined via methods known in the art. Generally speaking, any blend of luminescent materials can be treated as if it were a single luminescent material, thus the ratio of luminescent materials in the blend can be adjusted to continue to meet a target CIE value for LED strings having different peak emission wavelengths. Luminescent materials can be tuned for the desired excitation in response to the selected LEDs used in the LED strings (101A/101B/101A′₁/101B′₁/101A′₂/101B′₂/101A′₃/101B′₃/101A′₄/101B′₄/101A′ₙ/101B′ₙ), which may have different peak emission wavelengths within the range of from about 360 nm to about 535 nm. Suitable methods for tuning the response of luminescent materials are known in the art and may include altering the concentrations of dopants within a phosphor, for example. In some implementations of the present disclosure, luminophoric mediums can be provided with combinations of two types of luminescent materials. The first type of luminescent material emits light at a peak emission between about 515 nm and about 590 nm in response to the associated LED string emission. The second type of luminescent material emits at a peak emission between about 590 nm and about 700 nm in response to the associated LED string emission. In some instances, the luminophoric mediums disclosed herein can be formed from a combination of at least one luminescent material of the first and second types described in this paragraph. In implementations, the luminescent materials of the first type can emit light at a peak emission at about 515 nm, 525 nm, 530 nm, 535 nm, 540 nm, 545 nm, 550 nm, 555 nm, 560 nm, 565 nm, 570 nm, 575 nm, 580 nm, 585 nm, or 590 nm in response to the associated LED string emission. In preferred implementations, the luminescent materials of the first type can emit light at a peak emission between about 520 nm to about 555 nm. In implementations, the luminescent materials of the second type can emit light at a peak emission at about 590 nm, about 595 nm, 600 nm, 605 nm, 610 nm, 615 nm, 620 nm, 625 nm, 630 nm, 635 nm, 640 nm, 645 nm, 650 nm, 655 nm, 670 nm, 675 nm, 680 nm, 685 nm, 690 nm, 695 nm, or 700 nm in response to the associated LED string emission. In preferred implementations, the luminescent materials of the first type can emit light at a peak emission between about 600 nm to about 670 nm. Some exemplary luminescent materials of the first and second type are disclosed elsewhere herein and referred to as Compositions A-F. Table 17 shows aspects of some exemplar luminescent materials and properties.

Blends of Compositions A-F can be used in luminophoric mediums (101A/101B/101A′₁/101B′₁/101A′₂/101B′₂/101A′₃/101B′₃/101A′₄/101B′₄/101A′ₙ/101B′ₙ) to create luminophoric mediums having the desired saturated color points when excited by their respective LED strings (101A/101B/101C/101D). In some implementations, one or more blends of one or more of Compositions A-F can be used to produce luminophoric mediums (102A/102B/102C/102D). In some preferred implementations, one or more of Compositions A, B, and D and one or more of Compositions C, E, and F can be combined to produce luminophoric mediums (101A/101B/101A′₁/101B′₁/101A′₂/101B′₂/101A′₃/101B′₃/101A′₄/101B′₄/101A′ₙ/101B′ₙ). In some preferred implementations, the encapsulant for luminophoric mediums (101A/101B/101A′₁/101B′₁/101A′₂/101B′₂/101A′₃/101B′₃/101A′₄/101B′₄/101A′ₙ/101B′ₙ) comprises a matrix material having density of about 1.1 mg/mm³ and refractive index of about 1.545 or from about 1.4 to about 1.6. In some implementations, Composition A can have a refractive index of about 1.82 and a particle size from about 18 micrometers to about 40 micrometers. In some implementations, Composition B can have a refractive index of about 1.84 and a particle size from about 13 micrometers to about 30 micrometers. In some implementations, Composition C can have a refractive index of about 1.8 and a particle size from about 10 micrometers to about 15 micrometers. In some implementations, Composition D can have a refractive index of about 1.8 and a particle size from about 10 micrometers to about 15 micrometers. Suitable phosphor materials for Compositions A, B, C, and D are commercially available from phosphor manufacturers such as Mitsubishi Chemical Holdings Corporation (Tokyo, Japan), Intematix Corporation (Fremont, Calif.), EMD Performance Materials of Merck KGaA (Darmstadt, Germany), and PhosphorTech Corporation (Kennesaw, Ga.).

In certain implementations, the luminophoric mediums can include luminescent materials that comprise one or more quantum materials. Throughout this specification, the term “quantum material” means any luminescent material that includes: a quantum dot; a quantum wire; or a quantum well. Some quantum materials may absorb and emit light at spectral power distributions having narrow wavelength ranges, for example, wavelength ranges having spectral widths being within ranges of between about 25 nanometers and about 50 nanometers. In examples, two or more different quantum materials may be included in a lumiphor, such that each of the quantum materials may have a spectral power distribution for light emissions that may not overlap with a spectral power distribution for light absorption of any of the one or more other quantum materials. In these examples, cross-absorption of light emissions among the quantum materials of the lumiphor may be minimized. Throughout this specification, the term “quantum dot” means: a nanocrystal made of semiconductor materials that are small enough to exhibit quantum mechanical properties, such that its excitons are confined in all three spatial dimensions. Throughout this specification, the term “quantum wire” means: an electrically conducting wire in which quantum effects influence the transport properties. Throughout this specification, the term “quantum well” means: a thin layer that can confine (quasi-)particles (typically electrons or holes) in the dimension perpendicular to the layer surface, whereas the movement in the other dimensions is not restricted.

### EXAMPLES

### General Simulation Method.

Exemplary first and second lighting channels, and lighting systems having pairs of first and second lighting channels, were simulated. For each lighting channel, LED strings and recipient luminophoric mediums with particular emissions were selected, and then spectral power distributions and various light rendering characteristics and circadian-stimulating energy characteristics were calculated. Ra, R9, R13, R15, LER, Rf, Rg, CLA, CS, EML, BLH factor, CAF, CER, COI, GAI, GAI15, GAIBB, and circadian-stimulating energy characteristics were calculated at each representative point. Characteristics and aspects of the spectral power distributions are shown in Tables 3-12 and FIGS. 9-16.

The calculations were performed with Scilab (Scilab Enterprises, Versailles, France), LightTools (Synopsis, Inc., Mountain View, Calif.), and custom software created using Python (Python Software Foundation, Beaverton, Oreg.). Each lighting channel was simulated with an LED emission spectrum and excitation and emission spectra of luminophoric medium(s). The luminophoric mediums can comprise luminescent compositions of phosphors, quantum dots, or combinations thereof, with simulations performed based on absorption/emission spectrums and particle sizes. The exemplary first lighting channels were simulated using spectra of LEDs having peak wavelengths of between about 440 nm and about 510 nm, such as a 450 nm peak wavelength blue LED, one or more LUXEON Z Color Line royal blue LEDs (product code LXZ1-PR01) of color bin codes 3, 4, 5, or 6 (Lumileds Holding B.V., Amsterdam, Netherlands), one or more LUXEON Z Color Line blue LEDs (LXZ1-PB01) of color bin code 1 or 2 (Lumileds Holding B.V., Amsterdam, Netherlands), one or more LUXEON royal blue LEDs (product code LXML-PR01 and LXML-PR02) of color bins 3, 4, 5, or 6 (Lumileds Holding B.V., Amsterdam, Netherlands), one or more LUXEON Rebel Blue LEDs (LXML-PB01, LXML-PB02) of color bins 1, 2, 3, 4, or 5 (Lumileds Holding B.V., Amsterdam, Netherlands), or one or more LUXEON Rebel Cyan LEDs (LXML-PE01) of color bins 1, 2, 3, 4, or 5 (Lumileds Holding B.V., Amsterdam, Netherlands), for example. The exemplary second lighting channels were simulated using spectra of LEDs having peak wavelengths of between about 380 nm and about 420 nm, such as one or more 410 nm peak wavelength violet LEDs, one or more LUXEON Z UV LEDs (product codes LHUV-0380-, LHUV-0385-, LHUV-0390-, LHUV-0395-, LHUV-0400-, LHUV-0405-, LHUV-0410-, LHUV-0415-, LHUV-0420-,) (Lumileds Holding B.V., Amsterdam, Netherlands), one or more LUXEON UV FC LEDs (product codes LxF3-U410) (Lumileds Holding B.V., Amsterdam, Netherlands), one or more LUXEON UV U LEDs (product code LHUV-0415-) (Lumileds Holding B.V., Amsterdam, Netherlands), for example. Similar LEDs from other manufacturers such as OSRAM GmbH and Cree, Inc. that provide a saturated output at the desired peak wavelengths could also be used.

The emission, excitation and absorption curves for phosphors and quantum dots are available from commercial manufacturers such as Mitsubishi Chemical Holdings Corporation (Tokyo, Japan), Intematix Corporation (Fremont, Calif.), EMD Performance Materials of Merck KGaA (Darmstadt, Germany), and PhosphorTech Corporation (Kennesaw, Ga.). The luminophoric mediums used in the first and second lighting channels were simulated as combinations of one or more of luminescent compositions as described more fully elsewhere herein. Those of skill in the art appreciate that various combinations of LEDs and luminescent blends can be combined to generate combined emissions with desired color points on the 1931 CIE chromaticity diagram and the desired spectral power distributions.

### Example 1

A lighting system was simulated having a first lighting channel having the characteristics shown as “4000K Ch1” in Tables 3, 5, 7, 9, 11, 12, and 15 and a second lighting channel having the characteristics shown as “2400K Ch1” in Tables 3, 4, 6, 8, 10, 12, and 15 and in FIG. 12. The values for EML slope and EML ratio for this pair of first and second lighting channels are shown in Tables 13 and 14. The first lighting channel can comprise an LED having a 450 nm peak wavelength and an associated luminophoric medium having one or more phosphors, quantum dots, or a mixture thereof. The second lighting channel can comprise an LED having a 410 nm peak wavelength and an associated luminophoric medium having one or more phosphors, quantum dots, or a mixture thereof.

### Example 2

A lighting system was simulated having a first lighting channel having the characteristics shown as “4000K Ch2” in Tables 3, 5, 7, 9, 11, 12, and 15 and in FIG. 13, and a second lighting channel having the characteristics shown as “2400K Ch1” in Tables 3, 4, 6, 8, 10, 12, and 15 and in FIG. 12. The values for EML slope and EML ratio for this pair of first and second lighting channels are shown in Tables 13 and 14. The first lighting channel can comprise an LED having a 450 nm peak wavelength and an associated luminophoric medium having one or more phosphors, quantum dots, or a mixture thereof. The second lighting channel can comprise an LED having a 410 nm peak wavelength and an associated luminophoric medium having one or more phosphors, quantum dots, or a mixture thereof.

### Example 3

A lighting system was simulated having a first lighting channel having the characteristics shown as “4000K Ch3” in Tables 3, 5, 7, 9, 11, 12, and 15 and in FIG. 14, and a second lighting channel having the characteristics shown as “2400K Ch1” in Tables 3, 4, 6, 8, 10, 12, and 15 and in FIG. 12. The values for EML slope and EML ratio for this pair of first and second lighting channels are shown in Tables 13 and 14. The first lighting channel can comprise an LED having a 450 nm peak wavelength and an associated luminophoric medium having one or more phosphors, quantum dots, or a mixture thereof. The second lighting channel can comprise an LED having a 410 nm peak wavelength and an associated luminophoric medium having one or more phosphors, quantum dots, or a mixture thereof.

### Example 4

A lighting system was simulated having a first lighting channel having the characteristics shown as “4000K Ch4” in Tables 3, 5, 7, 9, 11, 12, and 15 and in FIG. 9, and a second lighting channel having the characteristics shown as “2400K Ch1” in Tables 3, 4, 6, 8, 10, 12, and 15 and in FIG. 12. The values for EML slope and EML ratio for this pair of first and second lighting channels are shown in Tables 13 and 14. The first lighting channel can comprise an LED having a 450 nm peak wavelength and an associated luminophoric medium having one or more phosphors, quantum dots, or a mixture thereof. The second lighting channel can comprise an LED having a 410 nm peak wavelength and an associated luminophoric medium having one or more phosphors, quantum dots, or a mixture thereof.

### Example 5

A lighting system was simulated having a first lighting channel having the characteristics shown as “5000K Ch1” in Tables 3, 5, 7, 9, 11, 12, and 15 and in FIG. 16, and a second lighting channel having the characteristics shown as “2400K Ch1” in Tables 3, 4, 6, 8, 10, 12, and 15 and in FIG. 12. The values for EML slope and EML ratio for this pair of first and second lighting channels are shown in Tables 13 and 14. The first lighting channel can comprise an LED having a 450 nm peak wavelength and an associated luminophoric medium having one or more phosphors, quantum dots, or a mixture thereof. The second lighting channel can comprise an LED having a 410 nm peak wavelength and an associated luminophoric medium having one or more phosphors, quantum dots, or a mixture thereof.

### Example 6

A lighting system was simulated having a first lighting channel having the characteristics shown as “4000K Ch1” in Tables 3, 5, 7, 9, 11, 12, and 15 and a second lighting channel having the characteristics shown as “2400K Ch2” in Tables 3, 4, 6, 8, 10, 12, and 15 and in FIG. 11. The values for EML slope and EML ratio for this pair of first and second lighting channels are shown in Tables 13 and 14. The first lighting channel can comprise an LED having a 450 nm peak wavelength and an associated luminophoric medium having one or more phosphors, quantum dots, or a mixture thereof. The second lighting channel can comprise an LED having a 410 nm peak wavelength and an associated luminophoric medium having one or more phosphors, quantum dots, or a mixture thereof.

### Example 7

A lighting system was simulated having a first lighting channel having the characteristics shown as “4000K Ch2” in Tables 3, 5, 7, 9, 11, 12, and 15 and in FIG. 13, and a second lighting channel having the characteristics shown as “2400K Ch2” in Tables 3, 4, 6, 8, 10, 12, and 15 and in FIG. 11. The values for EML slope and EML ratio for this pair of first and second lighting channels are shown in Tables 13 and 14. The first lighting channel can comprise an LED having a 450 nm peak wavelength and an associated luminophoric medium having one or more phosphors, quantum dots, or a mixture thereof. The second lighting channel can comprise an LED having a 410 nm peak wavelength and an associated luminophoric medium having one or more phosphors, quantum dots, or a mixture thereof.

### Example 8

A lighting system was simulated having a first lighting channel having the characteristics shown as “4000K Ch3” in Tables 3, 5, 7, 9, 11, 12, and 15 and in FIG. 14, and a second lighting channel having the characteristics shown as “2400K Ch2” in Tables 3, 4, 6, 8, 10, 12, and 15 and in FIG. 11. The values for EML slope and EML ratio for this pair of first and second lighting channels are shown in Tables 13 and 14. The first lighting channel can comprise an LED having a 450 nm peak wavelength and an associated luminophoric medium having one or more phosphors, quantum dots, or a mixture thereof. The second lighting channel can comprise an LED having a 410 nm peak wavelength and an associated luminophoric medium having one or more phosphors, quantum dots, or a mixture thereof.

### Example 9

A lighting system was simulated having a first lighting channel having the characteristics shown as “4000K Ch4” in Tables 3, 5, 7, 9, 11, 12, and 15 and in FIG. 9, and a second lighting channel having the characteristics shown as “2400K Ch2” in Tables 3, 4, 6, 8, 10, 12, and 15 and in FIG. 11. The values for EML slope and EML ratio for this pair of first and second lighting channels are shown in Tables 13 and 14. The first lighting channel can comprise an LED having a 450 nm peak wavelength and an associated luminophoric medium having one or more phosphors, quantum dots, or a mixture thereof. The second lighting channel can comprise an LED having a 410 nm peak wavelength and an associated luminophoric medium having one or more phosphors, quantum dots, or a mixture thereof.

### Example 10

A lighting system was simulated having a first lighting channel having the characteristics shown as “5000K Ch1” in Tables 3, 5, 7, 9, 11, 12, and 15 and in FIG. 16, and a second lighting channel having the characteristics shown as “2400K Ch2” in Tables 3, 4, 6, 8, 10, 12, and 15 and in FIG. 11. The values for EML slope and EML ratio for this pair of first and second lighting channels are shown in Tables 13 and 14. The first lighting channel can comprise an LED having a 450 nm peak wavelength and an associated luminophoric medium having one or more phosphors, quantum dots, or a mixture thereof. The second lighting channel can comprise an LED having a 410 nm peak wavelength and an associated luminophoric medium having one or more phosphors, quantum dots, or a mixture thereof.

### Example 11

A lighting system was simulated having a first lighting channel having the characteristics shown as “4000K Ch1” in Tables 3, 5, 7, 9, 11, 12, and 15 and a second lighting channel having the characteristics shown as “2400K Ch3” in Tables 3, 4, 6, 8, 10, 12, and 15 and in FIG. 10. The values for EML slope and EML ratio for this pair of first and second lighting channels are shown in Tables 13 and 14. The first lighting channel can comprise an LED having a 450 nm peak wavelength and an associated luminophoric medium having one or more phosphors, quantum dots, or a mixture thereof. The second lighting channel can comprise an LED having a 410 nm peak wavelength and an associated luminophoric medium having one or more phosphors, quantum dots, or a mixture thereof.

### Example 12

A lighting system was simulated having a first lighting channel having the characteristics shown as “4000K Ch2” in Tables 3, 5, 7, 9, 11, 12, and 15 and in FIG. 13, and a second lighting channel having the characteristics shown as “2400K Ch3” in Tables 3, 4, 6, 8, 10, 12, and 15 and in FIG. 10. The values for EML slope and EML ratio for this pair of first and second lighting channels are shown in Tables 13 and 14. The first lighting channel can comprise an LED having a 450 nm peak wavelength and an associated luminophoric medium having one or more phosphors, quantum dots, or a mixture thereof. The second lighting channel can comprise an LED having a 410 nm peak wavelength and an associated luminophoric medium having one or more phosphors, quantum dots, or a mixture thereof.

### Example 13

A lighting system was simulated having a first lighting channel having the characteristics shown as “4000K Ch3” in Tables 3, 5, 7, 9, 11, 12, and 15 and in FIG. 14, and a second lighting channel having the characteristics shown as “2400K Ch3” in Tables 3, 4, 6, 8, 10, 12, and 15 and in FIG. 10. The values for EML slope and EML ratio for this pair of first and second lighting channels are shown in Tables 13 and 14. The first lighting channel can comprise an LED having a 450 nm peak wavelength and an associated luminophoric medium having one or more phosphors, quantum dots, or a mixture thereof. The second lighting channel can comprise an LED having a 410 nm peak wavelength and an associated luminophoric medium having one or more phosphors, quantum dots, or a mixture thereof.

### Example 14

A lighting system was simulated having a first lighting channel having the characteristics shown as “4000K Ch4” in Tables 3, 5, 7, 9, 11, 12, and 15 and in FIG. 9, and a second lighting channel having the characteristics shown as “2400K Ch3” in Tables 3, 4, 6, 8, 10, 12, and 15 and in FIG. 10. The values for EML slope and EML ratio for this pair of first and second lighting channels are shown in Tables 13 and 14. The first lighting channel can comprise an LED having a 450 nm peak wavelength and an associated luminophoric medium having one or more phosphors, quantum dots, or a mixture thereof. The second lighting channel can comprise an LED having a 410 nm peak wavelength and an associated luminophoric medium having one or more phosphors, quantum dots, or a mixture thereof.

### Example 15

A lighting system was simulated having a first lighting channel having the characteristics shown as “5000K Ch1” in Tables 3, 5, 7, 9, 11, 12, and 15 and in FIG. 16, and a second lighting channel having the characteristics shown as “2400K Ch3” in Tables 3, 4, 6, 8, 10, 12, and 15 and in FIG. 10. The values for EML slope and EML ratio for this pair of first and second lighting channels are shown in Tables 13 and 14. The first lighting channel can comprise an LED having a 450 nm peak wavelength and an associated luminophoric medium having one or more phosphors, quantum dots, or a mixture thereof. The second lighting channel can comprise an LED having a 410 nm peak wavelength and an associated luminophoric medium having one or more phosphors, quantum dots, or a mixture thereof.

### Example 16

A lighting system was simulated having a first lighting channel having the characteristics shown as “4000K Ch1” in Tables 3, 5, 7, 9, 11, 12, and 15 and a second lighting channel having the characteristics shown as “1800K Ch1” in Tables 3, 4, 6, 8, 10, 12, and 15 and in FIG. 15. The values for EML slope and EML ratio for this pair of first and second lighting channels are shown in Tables 13 and 14. The first lighting channel can comprise an LED having a 450 nm peak wavelength and an associated luminophoric medium having one or more phosphors, quantum dots, or a mixture thereof. The second lighting channel can comprise an LED having a 410 nm peak wavelength and an associated luminophoric medium having one or more phosphors, quantum dots, or a mixture thereof.

### Example 17

A lighting system was simulated having a first lighting channel having the characteristics shown as “4000K Ch2” in Tables 3, 5, 7, 9, 11, 12, and 15 and in FIG. 13, and a second lighting channel having the characteristics shown as “1800K Ch1” in Tables 3, 4, 6, 8, 10, 12, and 15 and in FIG. 15. The values for EML slope and EML ratio for this pair of first and second lighting channels are shown in Tables 13 and 14. The first lighting channel can comprise an LED having a 450 nm peak wavelength and an associated luminophoric medium having one or more phosphors, quantum dots, or a mixture thereof. The second lighting channel can comprise an LED having a 410 nm peak wavelength and an associated luminophoric medium having one or more phosphors, quantum dots, or a mixture thereof.

### Example 18

A lighting system was simulated having a first lighting channel having the characteristics shown as “4000K Ch3” in Tables 3, 5, 7, 9, 11, 12, and 15 and in FIG. 14, and a second lighting channel having the characteristics shown as “1800K Ch1” in Tables 3, 4, 6, 8, 10, 12, and 15 and in FIG. 15. The values for EML slope and EML ratio for this pair of first and second lighting channels are shown in Tables 13 and 14. The first lighting channel can comprise an LED having a 450 nm peak wavelength and an associated luminophoric medium having one or more phosphors, quantum dots, or a mixture thereof. The second lighting channel can comprise an LED having a 410 nm peak wavelength and an associated luminophoric medium having one or more phosphors, quantum dots, or a mixture thereof.

### Example 19

A lighting system was simulated having a first lighting channel having the characteristics shown as “4000K Ch4” in Tables 3, 5, 7, 9, 11, 12, and 15 and in FIG. 9, and a second lighting channel having the characteristics shown as “1800K Ch1” in Tables 3, 4, 6, 8, 10, 12, and 15 and in FIG. 15. The values for EML slope and EML ratio for this pair of first and second lighting channels are shown in Tables 13 and 14. The first lighting channel can comprise an LED having a 450 nm peak wavelength and an associated luminophoric medium having one or more phosphors, quantum dots, or a mixture thereof. The second lighting channel can comprise an LED having a 410 nm peak wavelength and an associated luminophoric medium having one or more phosphors, quantum dots, or a mixture thereof.

### Example 20

A lighting system was simulated having a first lighting channel having the characteristics shown as “5000K Ch1” in Tables 3, 5, 7, 9, 11, 12, and 15 and in FIG. 16, and a second lighting channel having the characteristics shown as “1800K Ch1” in Tables 3, 4, 6, 8, 10, 12, and 15 and in FIG. 15. The values for EML slope and EML ratio for this pair of first and second lighting channels are shown in Tables 13 and 14. The first lighting channel can comprise an LED having a 450 nm peak wavelength and an associated luminophoric medium having one or more phosphors, quantum dots, or a mixture thereof. The second lighting channel can comprise an LED having a 410 nm peak wavelength and an associated luminophoric medium having one or more phosphors, quantum dots, or a mixture thereof.

### Example 21

A lighting system was simulated having a first lighting channel having the characteristics shown as “5000K Ch1” in Tables 3, 5, 7, 9, 11, 12, and 15 and in FIG. 16, and a second lighting channel having the characteristics shown as “1800K Ch1” in Tables 3, 4, 6, 8, 10, 12, and 15 and in FIG. 15. The values for EML slope and EML ratio for this pair of first and second lighting channels are shown in Tables 13 and 14. The first lighting channel can comprise an LED having a 450 nm peak wavelength and an associated luminophoric medium having one or more phosphors, quantum dots, or a mixture thereof. The second lighting channel can comprise an LED having a 410 nm peak wavelength and an associated luminophoric medium having one or more phosphors, quantum dots, or a mixture thereof.

### Example 22

A lighting system was simulated having a first lighting channel having the characteristics shown as “Exemplary 1st channels avg” in Tables 3, 5, 7, 9, 11, 12, and 15, and a second lighting channel having the characteristics shown as “Exemplary 2nd channels avg” in Tables 3, 4, 6, 8, 10, 12, and 15. The first lighting channel has a first color point at (0.3735, 0.3719) ccx, ccy coordinates. The second lighting channel has a second color point at (0.5021, 0.4137) ccx, ccy coordinates. The first lighting channel can comprise an LED having a 450 nm peak wavelength and an associated luminophoric medium having one or more phosphors, quantum dots, or a mixture thereof. The second lighting channel can comprise an LED having a 410 nm peak wavelength and an associated luminophoric medium having one or more phosphors, quantum dots, or a mixture thereof.

TABLE 1
EML performance for commercially-available
fixed-CCT LED lighting systems

| CCT | EML |
|---|---|
| 6500K | 1.350 |
| 5000K | 1.066 |
| 4000K | 0.935 |
| 3500K | 0.807 |
| 3000K | 0.690 |
| 2700K | 0.665 |
| 2500K | 0.603 |
| 2400K | 0.572 |

TABLE 2
EML performance for commercially-available
fixed-CCT LED lighting systems

| CCT | EML |
|---|---|
| 2200K | 0.347999 |
| 2400K | 0.3654583 |
| 2500K | 0.37418788 |
| 2700K | 0.4134712 |
| 3000K | 0.4675737 |
| 3500K | 0.5702719 |
| 4000K | 0.6567719 |
| 5000K | 0.7748765 |
| 5700K | 0.8203205 |
| 6500K | 0.9977367 |

TABLE 3

| | x | y | CCT | duv | Ra | R9 | R13 | R15 | LER | COI | GAI | GAI 15 | GAI_BB |
|---|---|---|---|---|---|---|---|---|---|---|---|---|---|
| 2400K Ch1 | 0.4872 | 0.4166 | 2401.7 | 0.62 | 76.39 | 50.16 | 81.3 | 61.64 | 312.32 | 10.53 | 36.61 | 89.03 | 83.17 |
| 2400K Ch2 | 0.4858 | 0.4148 | 2404.69 | 0.07 | 86.38 | 92.09 | 95.28 | 89.70 | 282.76 | 9.68 | 44.51 | 102.45 | 95.46 |
| 2400K Ch3 | 0.4852 | 0.4137 | 2403.72 | −0.29 | 80.60 | 35.83 | 84.04 | 81.58 | 282.07 | 7.79 | 41.87 | 100.73 | 93.95 |
| 1800K Ch1 | 0.5503 | 0.4097 | 1801 | 0.49 | 90.94 | 62.65 | 92.01 | 87.32 | 210.12 | 16.00 | 17.37 | 47.81 | 94.05 |
| 4000K Ch1 | 0.3807 | 0.3772 | 3995.74 | 0.16 | 91.18 | 58.05 | 90.71 | 86.30 | 292.50 | | 82.78 | 219.40 | 105.73 |
| 4000K Ch2 | 0.3803 | 0.3766 | 4003.12 | −0.02 | 88.67 | 96.86 | 89.72 | 94.57 | 274.59 | 1.2 | 76.69 | 200.10 | 96.28 |
| 4000K Ch3 | 0.3814 | 0.3758 | 3967.48 | −0.7 | 86.26 | 70.93 | 95.39 | 93.30 | 283.64 | 3.07 | 71.86 | 189.40 | 91.81 |
| 4000K Ch4 | 0.3804 | 0.3782 | 4012.69 | 0.72 | 82.45 | 79.82 | 91.17 | 92.69 | 280.02 | 2.4 | 69.51 | 182.68 | 87.72 |
| 5000K Ch1 | 0.3449 | 0.3516 | 5007 | 0.08 | 83.73 | 56.73 | 82.41 | 82.71 | 257.55 | 0.81 | 90.61 | 234.15 | 96.76 |

| | | | | | | | | | | | Energy |
|---|---|---|---|---|---|---|---|---|---|---|---|
| | | | CER | | | | | | | | in 440 < |
| | | | (Circadian | CAF | | Circadian | | | | | λ ≤ 490 nm/ |
| | Circadian | | power per | (Circadian | | Light (CLA) | Circadian | | | | total energy |
| | power | Circadian | flux) | action | | [Circadian | Stimulus | | | | 380 < λ ≤ |
| | [mW] | flux | [mW/lm] | factor | EML | lux] | (CS) | Rf | Rg | BLH | 780 nm |
| 2400K Ch1 | 0.0463 | 0.0074 | 77.736 | 0.2481 | 0.30848 | 575 | 0.440 | 51 | 97 | 0.10961 | 1.04% |
| 2400K Ch2 | 0.0294 | 0.0047 | 75.434 | 0.2661 | 0.34238 | 631 | 0.457 | 56 | 109 | 0.06700 | 0.99% |
| 2400K Ch3 | 0.0442 | 0.0065 | 69.309 | 0.2453 | 0.28563 | 540 | 0.429 | 51 | 103 | 0.10573 | 0.92% |
| 1800K Ch1 | 0.0265 | 0.0032 | 26.837 | 0.1209 | 0.21275 | 374 | 0.360 | 77 | 103 | 0.02570 | 0.98% |
| 4000K Ch1 | 0.0725 | 0.0241 | 174.436 | 0.5949 | 0.79451 | 767 | 0.490 | 91 | 102 | 0.20390 | 15.87% |
| 4000K Ch2 | 0.1042 | 0.0367 | 178.778 | 0.6494 | 0.88924 | 875 | 0.511 | 85 | 96 | 0.28816 | 15.97% |
| 4000K Ch3 | 0.0930 | 0.0331 | 184.994 | 0.6516 | 0.89470 | 896 | 0.514 | 80 | 91 | 0.25199 | 18.10% |
| 4000K Ch4 | 0.0847 | 0.0307 | 188.638 | 0.6729 | 0.94619 | 938 | 0.521 | 74 | 87 | 0.22073 | 18.56% |
| 5000K Ch1 | 0.0916 | 0.0355 | 215.982 | 0.8368 | 1.10190 | 1325 | 0.567 | 81 | 97 | 0.28801 | 21.00% |

TABLE 4

| | 320 < | 400 < | 500 < | 600 < | 700 < |
|---|---|---|---|---|---|
| | λ ≤ | λ ≤ | λ ≤ | λ ≤ | λ ≤ |
| | 400 | 500 | 600 | 700 | 780 |
| 2400K Ch3 | 9.92 | 44.53 | 83.33 | 100.00 | 7.55 |
| 2400K Ch2 | 8.59 | 39.69 | 75.82 | 100.00 | 3.09 |
| 2400K Ch1 | 11.11 | 51.02 | 105.53 | 100.00 | 4.41 |
| 1800K Ch1 | 7.61 | 4.42 | 39.66 | 100.00 | 11.52 |
| Exemplary 2^{nd} | 7.61 | 4.42 | 39.66 | 100.00 | 3.09 |
| channels min | | | | | |
| Exemplary 2^{nd} | 9.31 | 34.92 | 76.09 | 100.00 | 6.64 |
| channels avg | | | | | |
| Exemplary 2^{nd} | 11.11 | 51.02 | 105.53 | 100.00 | 11.52 |
| channels max | | | | | |

TABLE 5

| | 320 < | 400 < | 500 < | 600 < | 700 < |
|---|---|---|---|---|---|
| | λ ≤ | λ ≤ | λ ≤ | λ ≤ | λ ≤ |
| | 400 | 500 | 600 | 700 | 780 |
| 4000K Ch4 | 0.29 | 67.46 | 100.00 | 96.08 | 9.60 |
| 4000K Ch2 | 0.43 | 62.49 | 100.00 | 99.55 | 12.19 |
| 4000K Ch3 | 0.24 | 64.82 | 100.00 | 93.88 | 9.61 |
| 5000K Ch1 | 0.05 | 84.60 | 100.00 | 99.73 | 10.20 |
| Exemplary 1^{st} | 0.05 | 62.49 | 100.00 | 93.88 | 9.60 |
| channels min | | | | | |
| Exemplary 1^{st} | 0.25 | 69.84 | 100.00 | 97.31 | 10.40 |
| channels avg | | | | | |
| Exemplary 1^{st} | 0.43 | 84.60 | 100.00 | 99.73 | 12.19 |
| channels max | | | | | |

TABLE 6

| | 320 < | 380 < | 420 < | 460 < | 500 < | 540 < | 580 < | 620 < | 660 < | 700 < | 740 < |
|---|---|---|---|---|---|---|---|---|---|---|---|
| | λ ≤ | λ ≤ | λ ≤ | λ ≤ | λ ≤ | λ ≤ | λ ≤ | λ ≤ | λ ≤ | λ ≤ | λ ≤ |
| | 380 | 420 | 460 | 500 | 540 | 580 | 620 | 660 | 700 | 740 | 780 |
| 2400K Ch3 | 0.87 | 75.85 | 20.20 | 2.50 | 36.53 | 75.23 | 99.16 | 100.00 | 23.78 | 10.04 | 3.74 |
| 2400K Ch2 | 0.61 | 53.09 | 14.11 | 3.40 | 35.58 | 51.81 | 62.20 | 100.00 | 9.75 | 3.44 | 1.12 |
| 2400K Ch1 | 1.37 | 120.36 | 31.99 | 6.89 | 72.41 | 110.44 | 227.23 | 100.00 | 21.24 | 7.89 | 3.50 |
| 1800K Ch1 | 1.23 | 16.50 | 4.14 | 1.92 | 16.29 | 33.63 | 66.28 | 100.00 | 60.07 | 17.91 | 4.88 |
| Exemplary 2^{nd} | 0.61 | 16.50 | 4.14 | 1.92 | 16.29 | 33.63 | 62.20 | 100.00 | 9.75 | 3.44 | 1.12 |
| channels min | | | | | | | | | | | |
| Exemplary 2^{nd} | 1.02 | 66.45 | 17.61 | 3.68 | 40.20 | 67.78 | 113.72 | 100.00 | 28.71 | 9.82 | 3.31 |
| channels avg | | | | | | | | | | | |
| Exemplary 2^{nd} | 1.37 | 120.36 | 31.99 | 6.89 | 72.41 | 110.44 | 227.23 | 100.00 | 60.07 | 17.91 | 4.88 |
| channels max | | | | | | | | | | | |

TABLE 7

| | 320 < | 380 < | 420 < | 460 < | 500 < | 540 < | 580 < | 620 < | 660 < | 700 < | 740 < |
|---|---|---|---|---|---|---|---|---|---|---|---|
| | λ ≤ | λ ≤ | λ ≤ | λ ≤ | λ ≤ | λ ≤ | λ ≤ | λ ≤ | λ ≤ | λ ≤ | λ ≤ |
| | 380 | 420 | 460 | 500 | 540 | 580 | 620 | 660 | 700 | 740 | 780 |
| 4000K Ch4 | 0.39 | 0.59 | 30.88 | 98.73 | 67.12 | 76.66 | 100.00 | 84.15 | 50.00 | 13.89 | 4.63 |
| 4000K Ch2 | 0.54 | 1.99 | 44.28 | 79.86 | 78.17 | 75.94 | 100.00 | 95.38 | 52.20 | 18.93 | 5.61 |
| 4000K Ch3 | 0.29 | 0.70 | 37.77 | 87.23 | 65.19 | 79.15 | 100.00 | 82.62 | 48.47 | 13.93 | 4.68 |
| 5000K Ch1 | 0.01 | 1.49 | 66.19 | 129.05 | 96.22 | 88.49 | 100.00 | 115.83 | 63.66 | 18.66 | 5.03 |
| Exemplary 1^{st} | 0.01 | 0.59 | 30.88 | 79.86 | 65.19 | 75.94 | 100.00 | 82.62 | 48.47 | 13.89 | 4.63 |
| channels min | | | | | | | | | | | |
| Exemplary 1^{st} | 0.31 | 1.19 | 44.78 | 98.72 | 76.68 | 80.06 | 100.00 | 94.49 | 53.58 | 16.35 | 4.99 |
| channels avg | | | | | | | | | | | |
| Exemplary 1^{st} | 0.54 | 1.99 | 66.19 | 129.05 | 96.22 | 88.49 | 100.00 | 115.83 | 63.66 | 18.93 | 5.61 |
| channels max | | | | | | | | | | | |

TABLE 8

| | 320 < | 340 < | 360 < | 380 < | 400 < | 420 < | 440 < | 460 < | 480 < | 500 < | 520 < | 540 < |
|---|---|---|---|---|---|---|---|---|---|---|---|---|
| | λ ≤ | λ ≤ | λ ≤ | λ ≤ | λ ≤ | λ ≤ | λ ≤ | λ ≤ | λ ≤ | λ ≤ | λ ≤ | λ ≤ |
| | 340 | 360 | 380 | 400 | 420 | 440 | 460 | 480 | 500 | 520 | 540 | 560 |
| 2400K Ch3 | 0.00 | 0.02 | 1.13 | 22.91 | 77.86 | 23.21 | 3.62 | 0.62 | 2.70 | 13.97 | 34.57 | 48.20 |
| 2400K Ch2 | 0.00 | 0.02 | 0.72 | 14.60 | 49.67 | 14.79 | 2.29 | 0.48 | 3.64 | 15.31 | 27.77 | 31.81 |
| 2400K Ch1 | 0.00 | 0.04 | 1.83 | 37.29 | 126.84 | 37.77 | 5.84 | 1.20 | 8.20 | 35.22 | 63.53 | 73.06 |
| 1800K Ch1 | 0.00 | 0.00 | 2.61 | 29.27 | 5.68 | 4.41 | 4.36 | 1.12 | 2.94 | 11.91 | 22.59 | 30.12 |
| Exemplary 2^{nd} | 0.00 | 0.00 | 0.72 | 14.60 | 5.68 | 4.41 | 2.29 | 0.48 | 2.70 | 11.91 | 22.59 | 30.12 |
| channels min | | | | | | | | | | | | |
| Exemplary 2^{nd} | 0.00 | 0.02 | 1.57 | 26.02 | 65.01 | 20.05 | 4.03 | 0.86 | 4.37 | 19.10 | 37.11 | 45.80 |
| channels avg | | | | | | | | | | | | |
| Exemplary 2^{nd} | 0.00 | 0.04 | 2.61 | 37.29 | 126.84 | 37.77 | 5.84 | 1.20 | 8.20 | 35.22 | 63.53 | 73.06 |
| channels max | | | | | | | | | | | | |

| | 560 < | 580 < | 600 < | 620 < | 640 < | 660 < | 680 < | 700 < | 720 < | 740 < | 760 < | 780 < |
|---|---|---|---|---|---|---|---|---|---|---|---|---|
| | λ ≤ | λ ≤ | λ ≤ | λ ≤ | λ ≤ | λ ≤ | λ ≤ | λ ≤ | λ ≤ | λ ≤ | λ ≤ | λ ≤ |
| | 580 | 600 | 620 | 640 | 660 | 680 | 700 | 720 | 740 | 760 | 780 | 800 |
| 2400K Ch3 | 51.74 | 53.62 | 78.10 | 100.00 | 32.85 | 18.99 | 12.60 | 8.19 | 5.15 | 3.09 | 1.87 | 0.00 |
| 2400K Ch2 | 30.90 | 29.60 | 45.70 | 100.00 | 21.06 | 7.47 | 4.33 | 2.66 | 1.50 | 0.86 | 0.50 | 0.00 |
| 2400K Ch1 | 77.56 | 122.68 | 187.19 | 100.00 | 36.37 | 18.26 | 10.70 | 6.70 | 4.06 | 2.73 | 2.05 | 0.00 |
| 1800K Ch1 | 41.10 | 60.43 | 79.94 | 100.00 | 111.79 | 80.54 | 46.67 | 24.94 | 12.99 | 6.82 | 3.52 | 0.00 |
| Exemplary 2^{nd} | 30.90 | 29.60 | 45.70 | 100.00 | 21.06 | 7.47 | 4.33 | 2.66 | 1.50 | 0.86 | 0.50 | 0.00 |
| channels min | | | | | | | | | | | | |
| Exemplary 2^{nd} | 50.33 | 66.58 | 97.73 | 100.00 | 50.52 | 31.32 | 18.58 | 10.62 | 5.93 | 3.38 | 1.99 | 0.00 |
| channels avg | | | | | | | | | | | | |
| Exemplary 2^{nd} | 77.56 | 122.68 | 187.19 | 100.00 | 111.79 | 80.54 | 46.67 | 24.94 | 12.99 | 6.82 | 3.52 | 0.00 |
| channels max | | | | | | | | | | | | |

TABLE 9

| | 320 < | 340 < | 360 < | 380 < | 400 < | 420 < | 440 < | 460 < | 480 < | 500 < | 520 < | 540 < |
|---|---|---|---|---|---|---|---|---|---|---|---|---|
| | λ ≤ | λ ≤ | λ ≤ | λ ≤ | λ ≤ | λ ≤ | λ ≤ | λ ≤ | λ ≤ | λ ≤ | λ ≤ | λ ≤ |
| | 340 | 360 | 380 | 400 | 420 | 440 | 460 | 480 | 500 | 520 | 540 | 560 |
| 4000K Ch4 | 0.00 | 0.27 | 0.38 | 0.30 | 0.69 | 5.32 | 46.45 | 65.52 | 100.00 | 61.95 | 50.58 | 58.48 |
| 4000K Ch2 | 0.00 | 0.42 | 0.66 | 0.65 | 3.29 | 22.60 | 65.24 | 58.44 | 100.00 | 82.69 | 72.40 | 71.27 |
| 4000K Ch3 | 0.00 | 0.21 | 0.33 | 0.33 | 0.98 | 10.07 | 60.41 | 62.79 | 100.00 | 64.55 | 57.10 | 67.65 |
| 5000K Ch1 | 0.00 | 0.00 | 0.01 | 0.14 | 1.81 | 22.85 | 63.41 | 68.18 | 100.00 | 67.44 | 57.95 | 57.60 |
| Exemplary 1^{st} | 0.00 | 0.00 | 0.01 | 0.14 | 0.69 | 5.32 | 46.45 | 58.44 | 100.00 | 61.95 | 50.58 | 57.60 |
| channels min | | | | | | | | | | | | |
| Exemplary 1^{st} | 0.00 | 0.23 | 0.34 | 0.35 | 1.69 | 15.21 | 58.88 | 63.73 | 100.00 | 69.16 | 59.51 | 63.75 |
| channels avg | | | | | | | | | | | | |
| Exemplary 1^{st} | 0.00 | 0.42 | 0.66 | 0.65 | 3.29 | 22.85 | 65.24 | 68.18 | 100.00 | 82.69 | 72.40 | 71.27 |
| channels max | | | | | | | | | | | | |

| | 560 < | 580 < | 600 < | 620 < | 640 < | 660 < | 680 < | 700 < | 720 < | 740 < | 760 < | 780 < |
|---|---|---|---|---|---|---|---|---|---|---|---|---|
| | λ ≤ | λ ≤ | λ ≤ | λ ≤ | λ ≤ | λ ≤ | λ ≤ | λ ≤ | λ ≤ | λ ≤ | λ ≤ | λ ≤ |
| | 580 | 600 | 620 | 640 | 660 | 680 | 700 | 720 | 740 | 760 | 780 | 800 |
| 4000K Ch4 | 70.03 | 82.09 | 85.56 | 75.93 | 65.14 | 58.42 | 25.41 | 14.66 | 8.62 | 4.90 | 2.85 | 0.00 |
| 4000K Ch2 | 79.40 | 93.63 | 104.78 | 102.56 | 86.67 | 63.02 | 40.55 | 23.98 | 13.58 | 7.34 | 3.79 | 0.00 |
| 4000K Ch3 | 80.05 | 92.00 | 94.61 | 83.41 | 70.77 | 62.40 | 28.04 | 16.34 | 9.66 | 5.52 | 3.22 | 0.00 |
| 5000K Ch1 | 57.72 | 62.16 | 68.16 | 75.24 | 75.70 | 52.76 | 30.19 | 15.99 | 8.33 | 4.35 | 2.21 | 0.00 |
| Exemplary 1^{st} | 57.72 | 62.16 | 68.16 | 75.24 | 65.14 | 52.76 | 25.41 | 14.66 | 8.33 | 4.35 | 2.21 | 0.00 |
| channels min | | | | | | | | | | | | |
| Exemplary 1^{st} | 71.80 | 82.47 | 88.28 | 84.29 | 74.57 | 59.15 | 31.05 | 17.74 | 10.05 | 5.53 | 3.02 | 0.00 |
| channels avg | | | | | | | | | | | | |
| Exemplary 1^{st} | 80.05 | 93.63 | 104.78 | 102.56 | 86.67 | 63.02 | 40.55 | 23.98 | 13.58 | 7.34 | 3.79 | 0.00 |
| channels max | | | | | | | | | | | | |

TABLE 10

| | 320 < | 330 < | 340 < | 350 < | 360 < | 270 < | 380 < | 390 < | 400 < | 410 < | 420 < | 430 < |
|---|---|---|---|---|---|---|---|---|---|---|---|---|
| | λ ≤ | λ ≤ | λ ≤ | λ ≤ | λ ≤ | λ ≤ | λ ≤ | λ ≤ | λ ≤ | λ ≤ | λ ≤ | λ ≤ |
| | 330 | 340 | 350 | 360 | 370 | 380 | 390 | 400 | 410 | 420 | 430 | 440 |
| 2400K Ch3 | 0.00 | 0.00 | 0.00 | 0.04 | 0.26 | 1.62 | 7.91 | 30.16 | 70.32 | 59.07 | 27.46 | 11.12 |
| 2400K Ch2 | 0.00 | 0.00 | 0.00 | 0.03 | 0.18 | 1.16 | 5.65 | 21.61 | 50.41 | 42.34 | 19.67 | 7.95 |
| 2400K Ch1 | 0.00 | 0.00 | 0.00 | 0.06 | 0.40 | 2.52 | 12.31 | 47.07 | 109.79 | 92.21 | 42.85 | 17.31 |
| 1800K Ch1 | 0.00 | 0.00 | 0.00 | 0.00 | 0.00 | 5.56 | 42.52 | 19.80 | 8.19 | 3.91 | 3.64 | 5.74 |
| Exemplary 2^{nd} | 0.00 | 0.00 | 0.00 | 0.00 | 0.00 | 1.16 | 5.65 | 19.80 | 8.19 | 3.91 | 3.64 | 5.74 |
| channels min | | | | | | | | | | | | |
| Exemplary 2^{nd} | 0.00 | 0.00 | 0.00 | 0.03 | 0.21 | 2.71 | 17.09 | 29.66 | 59.67 | 49.38 | 23.40 | 10.53 |
| channels avg | | | | | | | | | | | | |
| Exemplary 2^{nd} | 0.00 | 0.00 | 0.00 | 0.06 | 0.40 | 5.56 | 42.52 | 47.07 | 109.79 | 92.21 | 42.85 | 17.31 |
| channels max | | | | | | | | | | | | |

| | 440 < | 450 < | 460 < | 470 < | 480 < | 490 < | 500 < | 510 < | 520 < | 530 < | 540 < | 550 < |
|---|---|---|---|---|---|---|---|---|---|---|---|---|
| | λ ≤ | λ ≤ | λ ≤ | λ ≤ | λ ≤ | λ ≤ | λ ≤ | λ ≤ | λ ≤ | λ ≤ | λ ≤ | λ ≤ |
| | 450 | 460 | 470 | 480 | 490 | 500 | 510 | 520 | 530 | 540 | 550 | 560 |
| 2400K Ch3 | 4.33 | 1.69 | 0.71 | 0.33 | 1.29 | 3.19 | 7.91 | 15.30 | 24.51 | 32.93 | 38.51 | 41.59 |
| 2400K Ch2 | 3.08 | 1.19 | 0.48 | 0.42 | 1.82 | 4.97 | 10.78 | 17.80 | 24.02 | 27.83 | 29.59 | 29.81 |
| 2400K Ch1 | 6.72 | 2.59 | 1.05 | 0.86 | 3.52 | 9.54 | 21.15 | 34.93 | 46.87 | 54.30 | 57.64 | 58.70 |
| 1800K Ch1 | 6.09 | 3.19 | 1.41 | 0.98 | 1.87 | 4.39 | 9.47 | 15.90 | 21.83 | 26.26 | 30.08 | 34.05 |
| Exemplary 2^{nd} | 3.08 | 1.19 | 0.48 | 0.33 | 1.29 | 3.19 | 7.91 | 15.30 | 21.83 | 26.26 | 29.59 | 29.81 |
| channels min | | | | | | | | | | | | |
| Exemplary 2^{nd} | 5.05 | 2.16 | 0.91 | 0.65 | 2.13 | 5.52 | 12.33 | 20.98 | 29.31 | 35.33 | 38.95 | 41.04 |
| channels avg | | | | | | | | | | | | |
| Exemplary 2^{nd} | 6.72 | 3.19 | 1.41 | 0.98 | 3.52 | 9.54 | 21.15 | 34.93 | 46.87 | 54.30 | 57.64 | 58.70 |
| channels max | | | | | | | | | | | | |

| | 560 < | 570 < | 580 < | 590 < | 600 < | 610 < | 620 < | 630 < | 640 < | 650 < | 660 < | 670 < |
|---|---|---|---|---|---|---|---|---|---|---|---|---|
| | λ ≤ | λ ≤ | λ ≤ | λ ≤ | λ ≤ | λ ≤ | λ ≤ | λ ≤ | λ ≤ | λ ≤ | λ ≤ | λ ≤ |
| | 570 | 580 | 590 | 600 | 610 | 620 | 630 | 640 | 650 | 660 | 670 | 680 |
| 2400K Ch3 | 42.83 | 43.16 | 43.36 | 45.76 | 54.76 | 75.03 | 100.00 | 66.18 | 32.55 | 22.04 | 17.39 | 14.17 |
| 2400K Ch2 | 29.31 | 28.39 | 27.39 | 27.87 | 32.98 | 52.35 | 100.00 | 86.71 | 27.42 | 11.91 | 7.93 | 6.02 |
| 2400K Ch1 | 59.43 | 64.08 | 79.86 | 115.51 | 152.93 | 145.17 | 100.00 | 59.25 | 35.20 | 22.72 | 16.72 | 12.37 |
| 1800K Ch1 | 39.62 | 47.89 | 58.66 | 70.02 | 80.33 | 89.88 | 100.00 | 112.92 | 122.48 | 115.54 | 96.01 | 75.48 |
| Exemplary 2^{nd} | 29.31 | 28.39 | 27.39 | 27.87 | 32.98 | 52.35 | 100.00 | 59.25 | 27.42 | 11.91 | 7.93 | 6.02 |
| channels min | | | | | | | | | | | | |
| Exemplary 2^{nd} | 42.80 | 45.88 | 52.32 | 64.79 | 80.25 | 90.61 | 100.00 | 81.26 | 54.41 | 43.05 | 34.51 | 27.01 |
| channels avg | | | | | | | | | | | | |
| Exemplary 2^{nd} | 59.43 | 64.08 | 79.86 | 115.51 | 152.93 | 145.17 | 100.00 | 112.92 | 122.48 | 115.54 | 96.01 | 75.48 |
| channels max | | | | | | | | | | | | |

| | 680 < | 690 < | 700 < | 710 < | 720 < | 730 < | 740 < | 750 < | 760 < | 770 < | 780 < | 790 < |
|---|---|---|---|---|---|---|---|---|---|---|---|---|
| | λ ≤ | λ ≤ | λ ≤ | λ ≤ | λ ≤ | λ ≤ | λ ≤ | λ ≤ | λ ≤ | λ ≤ | λ ≤ | λ ≤ |
| | 690 | 700 | 710 | 720 | 730 | 740 | 750 | 760 | 770 | 780 | 790 | 800 |
| 2400K Ch3 | 11.56 | 9.37 | 7.53 | 6.08 | 4.81 | 3.75 | 2.94 | 2.20 | 1.81 | 1.30 | 0.00 | 0.00 |
| 2400K Ch2 | 4.55 | 3.54 | 2.81 | 2.16 | 1.65 | 1.15 | 0.87 | 0.74 | 0.57 | 0.37 | 0.00 | 0.00 |
| 2400K Ch1 | 9.41 | 7.63 | 5.86 | 4.81 | 3.55 | 2.92 | 2.37 | 1.97 | 2.06 | 1.20 | 0.00 | 0.00 |
| 1800K Ch1 | 57.09 | 42.29 | 30.76 | 22.34 | 16.16 | 11.50 | 8.40 | 6.13 | 4.49 | 3.01 | 0.00 | 0.00 |
| Exemplary 2^{nd} | 4.55 | 3.54 | 2.81 | 2.16 | 1.65 | 1.15 | 0.87 | 0.74 | 0.57 | 0.37 | 0.00 | 0.00 |
| channels min | | | | | | | | | | | | |
| Exemplary 2^{nd} | 20.65 | 15.71 | 11.74 | 8.85 | 6.54 | 4.83 | 3.64 | 2.76 | 2.23 | 1.47 | 0.00 | 0.00 |
| channels avg | | | | | | | | | | | | |
| Exemplary 2^{nd} | 57.09 | 42.29 | 30.76 | 22.34 | 16.16 | 11.50 | 8.40 | 6.13 | 4.49 | 3.01 | 0.00 | 0.00 |
| channels max | | | | | | | | | | | | |

TABLE 11

| | 320 < | 330 < | 340 < | 350 < | 360 < | 370 < | 380 < | 390 < | 400 < | 410 < | 420 < | 430 < |
|---|---|---|---|---|---|---|---|---|---|---|---|---|
| | λ ≤ | λ ≤ | λ ≤ | λ ≤ | λ ≤ | λ ≤ | λ ≤ | λ ≤ | λ ≤ | λ ≤ | λ ≤ | λ ≤ |
| | 330 | 340 | 350 | 360 | 370 | 380 | 390 | 400 | 410 | 420 | 430 | 440 |
| 4000K Ch4 | 0.00 | 0.00 | 0.05 | 0.48 | 0.41 | 0.33 | 0.28 | 0.31 | 0.46 | 0.90 | 2.35 | 8.04 |
| 4000K Ch2 | 0.00 | 0.00 | 0.08 | 0.78 | 0.70 | 0.63 | 0.60 | 0.71 | 1.61 | 5.09 | 13.75 | 32.22 |
| 4000K Ch3 | 0.00 | 0.00 | 0.04 | 0.37 | 0.34 | 0.30 | 0.29 | 0.35 | 0.58 | 1.35 | 4.22 | 15.53 |
| 5000K Ch1 | 0.00 | 0.00 | 0.00 | 0.00 | 0.00 | 0.02 | 0.17 | 0.15 | 0.35 | 1.50 | 6.59 | 25.07 |
| Exemplary 1^{st} | 0.00 | 0.00 | 0.00 | 0.00 | 0.00 | 0.02 | 0.17 | 0.15 | 0.35 | 0.90 | 2.35 | 8.04 |
| channels min | | | | | | | | | | | | |
| Exemplary 1^{st} | 0.00 | 0.00 | 0.04 | 0.41 | 0.36 | 0.32 | 0.34 | 0.38 | 0.75 | 2.21 | 6.73 | 20.22 |
| channels avg | | | | | | | | | | | | |
| Exemplary 1^{st} | 0.00 | 0.00 | 0.08 | 0.78 | 0.70 | 0.63 | 0.60 | 0.71 | 1.61 | 5.09 | 13.75 | 32.22 |
| channels max | | | | | | | | | | | | |

| | 440 < | 450 < | 460 < | 470 < | 480 < | 490 < | 500 < | 510 < | 520 < | 530 < | 540 < | 550 < |
|---|---|---|---|---|---|---|---|---|---|---|---|---|
| | λ ≤ | λ ≤ | λ ≤ | λ ≤ | λ ≤ | λ ≤ | λ ≤ | λ ≤ | λ ≤ | λ ≤ | λ ≤ | λ ≤ |
| | 450 | 460 | 470 | 480 | 490 | 500 | 510 | 520 | 530 | 540 | 550 | 560 |
| 4000K Ch4 | 29.43 | 61.23 | 58.55 | 69.32 | 100.00 | 95.19 | 68.51 | 52.40 | 48.14 | 50.59 | 54.63 | 59.52 |
| 4000K Ch2 | 63.32 | 69.39 | 51.86 | 67.01 | 100.00 | 103.41 | 88.51 | 79.68 | 74.88 | 72.39 | 71.61 | 73.35 |
| 4000K Ch3 | 49.12 | 69.39 | 55.36 | 67.81 | 100.00 | 96.18 | 70.57 | 56.06 | 53.84 | 58.18 | 63.52 | 69.19 |
| 5000K Ch1 | 83.73 | 95.32 | 87.91 | 118.29 | 100.00 | 89.11 | 79.33 | 76.46 | 75.98 | 76.69 | 76.73 | 75.98 |
| Exemplary 1^{st} | 29.43 | 61.23 | 51.86 | 67.01 | 100.00 | 89.11 | 68.51 | 52.40 | 48.14 | 50.59 | 54.63 | 59.52 |
| channels min | | | | | | | | | | | | |
| Exemplary 1^{st} | 56.40 | 73.83 | 63.42 | 80.61 | 100.00 | 95.97 | 76.73 | 66.15 | 63.21 | 64.46 | 66.62 | 69.51 |
| channels avg | | | | | | | | | | | | |
| Exemplary 1^{st} | 83.73 | 95.32 | 87.91 | 118.29 | 100.00 | 103.41 | 88.51 | 79.68 | 75.98 | 76.69 | 76.73 | 75.98 |
| channels max | | | | | | | | | | | | |

| | 560 < | 570 < | 580 < | 590 < | 600 < | 610 < | 620 < | 630 < | 640 < | 650 < | 660 < | 670 < |
|---|---|---|---|---|---|---|---|---|---|---|---|---|
| | λ ≤ | λ ≤ | λ ≤ | λ ≤ | λ ≤ | λ ≤ | λ ≤ | λ ≤ | λ ≤ | λ ≤ | λ ≤ | λ ≤ |
| | 570 | 580 | 590 | 600 | 610 | 620 | 630 | 640 | 650 | 660 | 670 | 680 |
| 4000K Ch4 | 65.20 | 71.49 | 77.71 | 82.52 | 84.37 | 82.62 | 77.44 | 70.77 | 64.70 | 62.44 | 66.81 | 47.21 |
| 4000K Ch2 | 77.63 | 83.86 | 91.48 | 98.97 | 105.06 | 108.06 | 106.77 | 101.86 | 93.61 | 82.68 | 70.31 | 57.88 |
| 4000K Ch3 | 75.29 | 81.76 | 87.93 | 92.55 | 94.00 | 91.61 | 85.60 | 78.02 | 71.00 | 67.84 | 71.44 | 50.98 |
| 5000K Ch1 | 74.89 | 75.31 | 77.41 | 79.89 | 82.43 | 84.24 | 87.47 | 94.30 | 100.45 | 94.12 | 77.38 | 60.02 |
| Exemplary 1^{st} | 65.20 | 71.49 | 77.41 | 79.89 | 82.43 | 82.62 | 77.44 | 70.77 | 64.70 | 62.44 | 66.81 | 47.21 |
| channels min | | | | | | | | | | | | |
| Exemplary 1^{st} | 73.25 | 78.10 | 83.63 | 88.48 | 91.47 | 91.63 | 89.32 | 86.24 | 82.44 | 76.77 | 71.48 | 54.02 |
| channels avg | | | | | | | | | | | | |
| Exemplary 1^{st} | 77.63 | 83.86 | 91.48 | 98.97 | 105.06 | 108.06 | 106.77 | 101.86 | 100.45 | 94.12 | 77.38 | 60.02 |
| channels max | | | | | | | | | | | | |

| | 680 < | 690 < | 700 < | 710 < | 720 < | 730 < | 740 < | 750 < | 760 < | 770 < | 780 < | 790 < |
|---|---|---|---|---|---|---|---|---|---|---|---|---|
| | λ ≤ | λ ≤ | λ ≤ | λ ≤ | λ ≤ | λ ≤ | λ ≤ | λ ≤ | λ ≤ | λ ≤ | λ ≤ | λ ≤ |
| | 690 | 700 | 710 | 720 | 730 | 740 | 750 | 760 | 770 | 780 | 790 | 800 |
| 4000K Ch4 | 28.56 | 21.04 | 16.09 | 12.52 | 9.54 | 7.29 | 5.50 | 4.07 | 3.17 | 2.40 | 0.00 | 0.00 |
| 4000K Ch2 | 46.42 | 36.07 | 27.73 | 21.05 | 15.78 | 11.85 | 8.63 | 6.29 | 4.50 | 3.21 | 0.00 | 0.00 |
| 4000K Ch3 | 31.57 | 23.45 | 18.00 | 14.05 | 10.74 | 8.22 | 6.22 | 4.61 | 3.61 | 2.72 | 0.00 | 0.00 |
| 5000K Ch1 | 45.22 | 33.23 | 24.15 | 17.38 | 12.51 | 8.98 | 6.52 | 4.67 | 3.43 | 2.24 | 0.00 | 0.00 |
| Exemplary 1^{st} | 28.56 | 21.04 | 16.09 | 12.52 | 9.54 | 7.29 | 5.50 | 4.07 | 3.17 | 2.24 | 0.00 | 0.00 |
| channels min | | | | | | | | | | | | |
| Exemplary 1^{st} | 37.94 | 28.44 | 21.49 | 16.25 | 12.14 | 9.08 | 6.72 | 4.91 | 3.68 | 2.64 | 0.00 | 0.00 |
| channels avg | | | | | | | | | | | | |
| Exemplary 1^{st} | 46.42 | 36.07 | 27.73 | 21.05 | 15.78 | 11.85 | 8.63 | 6.29 | 4.50 | 3.21 | 0.00 | 0.00 |
| channels max | | | | | | | | | | | | |

TABLE 12

| | 400 < | 470 < | 530 < | 600 < | 630 < |
|---|---|---|---|---|---|
| | λ ≤ | λ ≤ | λ ≤ | λ ≤ | λ ≤ |
| | 470 nm | 510 nm | 570 nm | 630 nm | 780 nm |
| 2400K Ch3 | 14.063 | 1.000 | 12.431 | 18.374 | 16.714 |
| 2400K Ch2 | 7.136 | 1.000 | 6.611 | 10.443 | 9.461 |
| 2400K Ch1 | 7.971 | 1.000 | 6.693 | 11.715 | 5.576 |
| 1800K Ch1 | 1.990 | 1.000 | 7.873 | 16.512 | 43.711 |
| Exemplary 2^{nd} | 1.990 | 1.000 | 6.611 | 10.443 | 5.576 |
| channels min | | | | | |
| Exemplary 2^{nd} | 7.790 | 1.000 | 8.402 | 14.261 | 18.866 |
| channels avg | | | | | |
| Exemplary 2^{nd} | 14.063 | 1.000 | 12.431 | 18.374 | 43.711 |
| channels max | | | | | |
| 4000K Ch4 | 0.475 | 1.000 | 0.693 | 0.746 | 1.268 |
| 4000K Ch2 | 0.652 | 1.000 | 0.830 | 0.906 | 1.643 |
| 4000K Ch3 | 0.575 | 1.000 | 0.799 | 0.825 | 1.385 |
| 5000K Ch1 | 0.634 | 1.000 | 0.652 | 0.596 | 1.493 |
| Exemplary 1^{st} | 0.475 | 1.000 | 0.652 | 0.596 | 1.268 |
| channels min | | | | | |
| Exemplary 1^{st} | 0.584 | 1.000 | 0.744 | 0.769 | 1.447 |
| channels avg | | | | | |
| Exemplary 1^{st} | 0.652 | 1.000 | 0.830 | 0.906 | 1.643 |
| channels max | | | | | |

TABLE 13
EML Slope vs. CCT (per 1000K) for Pairings
of Exemplary First/Second Lighting Channels

| | 4000K | 4000K | 4000K | 4000K | 5000K |
|---|---|---|---|---|---|
| | Ch1 | Ch2 | Ch3 | Ch4 | Ch1 |
| 2400K Ch1 | 0.305 | 0.363 | 0.374 | 0.396 | 0.305 |
| 2400K Ch2 | 0.284 | 0.342 | 0.353 | 0.375 | 0.292 |
| 2400K Ch3 | 0.320 | 0.377 | 0.389 | 0.411 | 0.314 |
| 1800K Ch1 | 0.265 | 0.307 | 0.315 | 0.332 | 0.277 |

TABLE 14
EML Ratio of First Lighting Channel to Second Lighting Channel
for Pairings of Exemplary First/Second Lighting Channels

| | 4000K | 4000K | 4000K | 4000K | 5000K |
|---|---|---|---|---|---|
| | Ch1 | Ch2 | Ch3 | Ch4 | Ch1 |
| 2400K Ch1 | 2.6 | 2.9 | 2.9 | 3.1 | 3.6 |
| 2400K Ch2 | 2.3 | 2.6 | 2.6 | 2.8 | 3.2 |
| 2400K Ch3 | 2.8 | 3.1 | 3.1 | 3.3 | 3.9 |
| 1800K Ch1 | 3.7 | 4.2 | 4.2 | 4.4 | 5.2 |

TABLE 15
% Spectral Energy in Wavelength Range vs. Total Energy 320 nm to 800 nm

| | 400 < | 410 < | 420 < | 430 < | 440 < | 450 < | 460 < | 470 < | 480 < | 490 < | 500 < | 510 < |
|---|---|---|---|---|---|---|---|---|---|---|---|---|
| | λ ≤ | λ ≤ | λ ≤ | λ ≤ | λ ≤ | λ ≤ | λ ≤ | λ ≤ | λ ≤ | λ ≤ | λ ≤ | λ ≤ |
| | 410 | 420 | 430 | 440 | 450 | 460 | 470 | 480 | 490 | 500 | 510 | 520 |
| 2400K Ch3 | 7.11 | 5.97 | 2.78 | 1.12 | 0.44 | 0.17 | 0.071 | 0.033 | 0.13 | 0.32 | 0.80 | 1.55 |
| 2400K Ch2 | 6.65 | 5.59 | 2.60 | 1.05 | 0.41 | 0.16 | 0.064 | 0.056 | 0.24 | 0.66 | 1.42 | 2.35 |
| 2400K Ch1 | 7.19 | 6.04 | 2.81 | 1.13 | 0.44 | 0.17 | 0.069 | 0.056 | 0.23 | 0.62 | 1.38 | 2.29 |
| 1800K Ch1 | 0.56 | 0.27 | 0.25 | 0.39 | 0.42 | 0.22 | 0.097 | 0.067 | 0.13 | 0.30 | 0.65 | 1.09 |
| Exemplary 2^{nd} | 0.56 | 0.27 | 0.25 | 0.39 | 0.41 | 0.16 | 0.064 | 0.033 | 0.13 | 0.30 | 0.65 | 1.09 |
| channels min | | | | | | | | | | | | |
| Exemplary 2^{nd} | 5.38 | 4.47 | 2.11 | 0.93 | 0.43 | 0.18 | 0.075 | 0.053 | 0.18 | 0.48 | 1.06 | 1.82 |
| channels avg | | | | | | | | | | | | |
| Exemplary 2^{nd} | 7.19 | 6.04 | 2.81 | 1.13 | 0.44 | 0.22 | 0.097 | 0.067 | 0.24 | 0.66 | 1.42 | 2.35 |
| channels max | | | | | | | | | | | | |
| 4000K Ch4 | 0.03 | 0.05 | 0.14 | 0.47 | 1.71 | 3.55 | 3.40 | 4.02 | 5.80 | 5.52 | 3.97 | 3.04 |
| 4000K Ch2 | 0.07 | 0.23 | 0.62 | 1.44 | 2.84 | 3.11 | 2.32 | 3.00 | 4.48 | 4.63 | 3.97 | 3.57 |
| 4000K Ch3 | 0.03 | 0.07 | 0.22 | 0.82 | 2.58 | 3.64 | 2.91 | 3.56 | 5.25 | 5.05 | 3.71 | 2.94 |
| 5000K Ch1 | 0.02 | 0.07 | 0.30 | 1.13 | 3.78 | 4.30 | 3.97 | 5.34 | 4.51 | 4.02 | 3.58 | 3.45 |
| Exemplary 1^{st} | 0.02 | 0.05 | 0.14 | 0.47 | 1.71 | 3.11 | 2.32 | 3.00 | 4.48 | 4.02 | 3.58 | 2.94 |
| channels min | | | | | | | | | | | | |
| Exemplary 1^{st} | 0.04 | 0.10 | 0.32 | 0.96 | 2.73 | 3.65 | 3.15 | 3.98 | 5.01 | 4.81 | 3.81 | 3.25 |
| channels avg | | | | | | | | | | | | |
| Exemplary 1^{st} | 0.07 | 0.23 | 0.62 | 1.44 | 3.78 | 4.30 | 3.97 | 5.34 | 5.80 | 5.52 | 3.97 | 3.57 |
| channels max | | | | | | | | | | | | |

TABLE 16

| ANSI | | | | | | | | | | |
|---|---|---|---|---|---|---|---|---|---|---|
| Nominal | | | | | | | | | | |
| CCT | Center | | Tolerance | | | | Boundaries | | | |
| Boundary | CCT | duv | dCCT | dduv | Center | | 1 | 2 | 3 | 4 |
| 2200 | 2238 | −0.0942 | ±102 | ±5.3 | Cx | 0.5018 | 0.4838 | 0.5046 | 0.5262 | 0.5025 |
| | | | | | Cy | 0.4153 | 0.3977 | 0.4007 | 0.4381 | 0.4348 |
| 2500 | 2470 | −0.3065 | ±109 | ±5.7 | Cx | 0.4792 | 0.4593 | 0.4838 | 0.5025 | 0.4813 |
| | | | | | Cy | 0.4131 | 0.3944 | 0.3977 | 0.4348 | 0.4319 |
| 2700 | 2725 | −0.0837 | ±145 | ±6.0 | Cx | 0.4578 | 0.4813 | 0.4562 | 0.4373 | 0.4593 |
| | | | | | Cy | 0.4101 | 0.4319 | 0.4260 | 0.3893 | 0.3944 |
| 3000 | 3045 | −0.0773 | ±175 | ±6.0 | Cx | 0.4338 | 0.4562 | 0.4299 | 0.4147 | 0.4373 |
| | | | | | Cy | 0.403 | 0.4260 | 0.4165 | 0.3814 | 0.3893 |
| 3500 | 3464 | −0.0698 | ±245 | ±6.0 | Cx | 0.4073 | 0.4299 | 0.3996 | 0.3889 | 0.4147 |
| | | | | | Cy | 0.3917 | 0.4165 | 0.4015 | 0.369 | 0.3814 |
| 4000 | 3985 | 0.9845 | ±275 | ±6.0 | Cx | 0.3818 | 0.4006 | 0.3736 | 0.3670 | 0.3898 |
| | | | | | Cy | 0.3797 | 0.4044 | 0.3874 | 0.3578 | 0.3716 |
| 5000 | 5027 | 2.0112 | ±283 | ±6.0 | Cx | 0.3447 | 0.3551 | 0.3376 | 0.3366 | 0.3515 |
| | | | | | Cy | 0.3553 | 0.376 | 0.3616 | 0.3369 | 0.3487 |
| 5700 | 5666 | 2.0235 | ±355 | ±6.0 | Cx | 0.3287 | 0.3376 | 0.3207 | 0.3222 | 0.3366 |
| | | | | | Cy | 0.3417 | 0.3616 | 0.3462 | 0.3243 | 0.3369 |
| 6500 | 6532 | 2.9989 | ±510 | ±6.0 | Cx | 0.3123 | 0.3205 | 0.3028 | 0.3068 | 0.3221 |
| | | | | | Cy | 0.3282 | 0.3481 | 0.3304 | 0.3113 | 0.3261 |

TABLE 17

| | | | | | Emission | |
|---|---|---|---|---|---|---|
| | | | Emission | | Peak | FWHM |
| | | Density | Peak | FWHM | Range | Range |
| Designator | Exemplary Material(s) | (g/mL) | (nm) | (nm) | (nm) | (nm) |
| Composition | Luag: Cerium doped | 6.73 | 535 | 95 | 530-540 | 90-100 |
| “A” | lutetium aluminum garnet | | | | | |
| | (Lu₃Al₅O₁₂) | | | | | |
| Composition | Yag: Cerium doped | 4.7 | 550 | 110 | 545-555 | 105-115 |
| “B” | yttrium aluminum garnet | | | | | |
| | (Y₃Al₅O₁₂) | | | | | |
| Composition | a 650 nm-peak wavelength | 3.1 | 650 | 90 | 645-655 | 85-95 |
| “C” | emission phosphor: | | | | | |
| | Europium doped calcium | | | | | |
| | aluminum silica nitride | | | | | |
| | (CaAlSiN₃) | | | | | |
| Composition | a 525 nm-peak wavelength | 3.1 | 525 | 60 | 520-530 | 55-65 |
| “D” | emission phosphor: | | | | | |
| | GBAM: BaMgAl₁₀O₁₇:Eu | | | | | |
| Composition | a 630 nm-peak wavelength | 5.1 | 630 | 40 | 625-635 | 35-45 |
| “E” | emission quantum | | | | | |
| | dot: any semiconductor | | | | | |
| | quantum dot material of | | | | | |
| | appropriate size for desired | | | | | |
| | emission wavelengths | | | | | |
| Composition | a 610 nm-peak wavelength | 5.1 | 610 | 40 | 605-615 | 35-45 |
| “F” | emission quantum | | | | | |
| | dot: any semiconductor | | | | | |
| | quantum dot material of | | | | | |
| | appropriate size for desired | | | | | |
| | emission wavelengths | | | | | |

Control Systems.

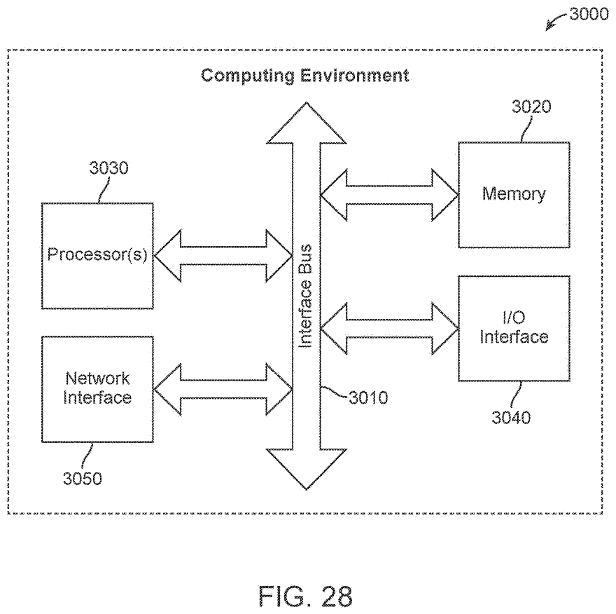

As illustrated in FIGS. 28-30, various lighting systems and control system exemplars an aspects thereof may be implemented in accordance with the present disclosure. Although aspects of methods, systems and devices are discussed below, but it will be appreciated that the disclosure is not limited to those particular configurations, and may be applied to any combination of devices, computing systems, control systems, data centers, structures, and the like.

At a simplified level aspects of the system and method disclosed herein include utilizing hardware referred to as computing or smart devices which may include internet streaming, desktop computers, laptops, tablets, smart phones, and sensors, to acquire, receive, measure or otherwise capture and then transmit via signal communication data associated with biological aspects of a user or data concerning the exposure of a user to variables discussed herein.

It is appreciated by those of ordinary skill in the art that some of the circuits, components, modules, and/or devices of the system disclosed in the present application are described as being in signal communication with each other, where signal communication refers to any type of communication and/or connection between the circuits, components, modules, and/or devices that allows a circuit, component, module, and/or device to pass and/or receive signals and/or information from another circuit, component, module, and/or device. The communication and/or connection may be along any signal path between the circuits, components, modules, and/or devices that allows signals and/or information to pass from one circuit, component, module, and/or device to another and includes wireless or wired signal paths. The signal paths may be physical such as, for example, conductive wires, electromagnetic wave guides, attached and/or electromagnetic or mechanically coupled terminals, semi-conductive or dielectric materials or devices, or other similar physical connections or couplings. Additionally, signal paths may be non-physical such as free-space (in the case of electromagnetic propagation) or information paths through digital components where communication information is passed from one circuit, component, module, and/or device to another in varying analog and/or digital formats without passing through a direct electromagnetic connection. These information paths may also include analog-to-digital conversions (“ADC”), digital-to-analog (“DAC”) conversions, data transformations such as, for example, fast Fourier transforms (“FFTs”), time-to-frequency conversations, frequency-to-time conversions, database mapping, signal processing steps, coding, modulations, demodulations, etc.

An integrated control system can connect one or more external systems, input, and information to provide bioactive lighting, as discussed herein, through a plurality of devices, systems, and modalities. In various examples, the control system may communicate over one or more computing systems using one or more servers and networks 3305 in communication with one another (e.g., network, Bluetooth, wired, wireless communication, etc.).

In some embodiments, lighting systems associated with each device may be managed by a master device 3340, configured to communicate various lighting levels, timing, and configuration, for example, to achieve the desired bioactive lighting. Such levels may vary based on one or more of time of day, intended effect of the lighting, individual preferences, capabilities of the device, feedback mechanisms, sensor input, and more.

Control systems may comprise a variety of devices, including but not limited to panels and panel systems 3310, computing systems 3320, laptops, mobile devices 3330, wearable devices 3333, sensors 3335, lighting systems 3350 including but not limited to home, office, vehicle, and industrial lighting systems. The master device 3340 may be a mobile device, computing systems, as discussed further below, and may be manually managed, automated, incorporated with machine learning, located in the cloud, and more.

In an example, lighting systems that may be used in a bioactive device including but not limited to wearable devices 3333, computer display system and/or bioactive panel system 3310 in accordance with the principles of the present disclosure may be controlled over time to supplement, treat or otherwise effect biological system and cycles of an exposed user throughout the day in different ways. The lighting systems may be automatically, semi-automatically or manually adjusted. The lighting systems may be adjusted based on sensor data, activity data, social media data, etc.

In some embodiments, as the panel 3310 systems are installed in the environment of a lighting installation, networking features automatically engage upon powering up one or more the panel systems, and the panel systems may automatically commission themselves, such as by connecting to an overall control platform and/or to other panel systems. Thus, the panel systems in an installation may self-commission and self-configure to create a network connection between the panel systems in the environment and a remote operator (such as in the cloud). The panel systems may configure in a master/slave, ring, mesh, or peer-to-peer network, by which autonomous control features may be engaged in the environment. In embodiments, remote control features may be engaged using the network connection to the platform or other remote operators.

In some embodiments, networked communication can be used among components in the control system 3000 in a deployed lighting installation that includes panel systems. Once installed and commissioned, control of the lighting installation may be handed over to an operator of a platform, such as a building owner, occupant, landlord, tenant, or the like. In embodiments, handoff may include using identity and authentication features, such as using keys, passwords, or the like that allow operation of the lighting installation by permitted users. In some embodiments, a remote-control interface of the platform may be used by an operator for remote operation of the lighting installation. The remote-control interface may use a lighting project data structure as a source of knowledge about the properties, configurations, control capabilities, and other elements of a lighting installation, so that the same platform used for the design of the lighting installation may be used to control the lighting installation. The remote-control interface may include operational guidance features, such as guiding users through the operation of a lighting installation.

In some embodiments, an autonomous control system may be provided for a lighting installation that includes panel systems of the present disclosure, by which the lighting installation may control various features of the lighting system, such as based on information collected locally in the environment, such as from one or more sensors 3330. For example, the autonomous control system may automatically adjust control parameters for a light source, including but not limited to panel systems, to achieve improved adherence to the overall specifications for a lighting installation, may adjust timing variables based on detected usage patterns in a space, may adjust lighting properties based on changes in a space (such as changes in colors paints, carpet and fabrics), and the like.

Under operation, the lighting installation may include an operational feedback system, configured to collect information about the lighting installation, which may include interfaces for soliciting and receiving user feedback (such as regarding satisfaction with the installation or indicating desired changes) and which may include a sensor system 3330, e.g., a lighting installation sensor system, such as including light sensors, motion sensors, temperature sensors, and others to collect information about the actual lighting conditions in the environment, activities of occupants within the environment, and the like. Information collected by the lighting installation sensor system may be relayed to a validation system of the lighting platform, such as for validation that an installation is operating as designed, including by comparison of light properties at various locations in the environment with the specifications and requirements provided in the lighting design environment, such as reflected in the lighting project data structure. In embodiments, the variances from the specifications and requirements may be provided to the autonomous control system and/or the remote-control system, so that adjustments may be made, either autonomously or by a local or remote operator of the lighting installation, to enable adjustments (such as to colors, intensities, color temperatures, beam directions, and other factors), such as to cause the lighting installation to better meet the specifications and requirements. The operational feedback system may also capture feedback that leads to revisiting the lighting design in the lighting design environment, which may induce further iteration, resulting in changes to control parameters for the panel systems, as well as automated ordering of additional or substitute panel systems, with updated installation and operational guidance.

In some embodiments, remote control may enable field programmable lighting systems, such as for transitional environments like museums (where art objects change regularly), stores (where merchandise shifts) and the like as well as for customizable environments (such as personalizing lighting in a hotel room according to a specification for a guest (which may include having the guest select an aesthetic filter) or personalized lighting for a workstation for an employee in an office setting, or personalized wearable systems. Such features may enable the lighting installation to change configurations (such as among different aesthetic filters) for multi-use environments, multi-tenant environments, and the like where lighting conditions may need to change substantially over time.

In some embodiments, a lighting system may include navigation features, such as being associated with beacons, where the lighting system interacts with one or more devices to track users within a space. The panel systems and their locations may be associated with a map, such as the map of the lighting space in the design environment. The map may be provided from the lighting design environment to one or more other location or navigation systems, such that locations of panel systems may be used as known locations or points of interest within a space.

In some embodiments, the lighting installation may be designed for an operation that is coordinated with one or more external systems, e.g., lighting, panel, and computer systems, which may serve as inputs to the lighting installation, such as music, video and other entertainment content (such as to coordinate lighting with sound). Inputs may include voice control inputs, which may include systems for assessing tone or mood from vocal patterns, such as to adjust lighting based on the same.

With respect to FIGS. 28-30 external systems can include, but are not limited to one or more computing environments, networks, local devices, remote devices, mobile devices, and wearable technology. In addition, each of those systems may provide the external input utilizable with control systems and embodiments discussed herein. For example, external inputs may include, but are not limited to audible, tactile, sensory, and user information through one or more sensors and other means, depending on the external system and its capabilities. As used herein, external systems and external information may also comprise the same types systems and information discussed below and in various embodiments herein.

In some embodiments, inputs may also include inputs from sensors associated with wearable devices 3330, such as enabling adjustment of lighting control parameters (autonomously or with remote or local control features) based on physiological factors, such as ones indicating health conditions, emotional states, moods, or the like. Inputs from wearable devices may be used in the operational feedback system, such as to measure reactions to lighting conditions (such as to enable automated adjustment of a lighting installation), as well as to measure impacts on mood, health conditions, energy, wellness factors, and the like.

In some embodiments, the platform may be configured to change settings or parameters for a lighting installation (including but not limited to panel systems of the present disclosure, such as by using a custom tuning system) based on a variety of real time data, with a view to having the lighting installation, including panel systems included therein, best suit its environment in a dynamic way. In embodiments, data may be obtained that serves as an indicator of the emotional state or the stress level of an environment, and the lighting installation may respond accordingly to that state or stress level. In embodiments, data about the environment may be collected by a wearable device 3333, such as a smartwatch, armband, or the like; for example, data may be collected on acceleration, location, ambient light characteristics, and heart rate, among other possibilities. In embodiments, the data may be provided to the platform for analysis, including using machine learning, such as to observe physiological indicators of stress, mood, or the like under given lighting conditions. The analysis may enable model-based controls (such as where a given mood or state of the users in a room are linked to a set of control parameters appropriate for that state). In embodiments, machine learning may be used; for example, over time, by variation of parameters for lighting objects and fixtures (such as color, color temperature, illumination patterns, lighting distributions, and many others), a machine learning system may, using feedback on outcomes based at least in part on physiological data and other data collected by a wearable device, select and/or promotion lighting installation parameters that improve various measures of stress, mood, satisfaction, or the like. This may occur in real time under control of a machine learning system based on the current conditions of users or the environment. In embodiments, data collected at least in part by a physiological monitor or wearable device may be used as an input to processing logic on a lighting object that changes lighting levels or other parameters to accommodate the ‘emotional state’ of the users in an environment where the lighting object is located. In embodiments, there is memory that retains and manages function with no appreciable drain on the battery.

In some embodiments, inputs may include systems that take data harvested from sensors 3335 in the lighting installation environment as well as sensors that reflect information about users, such as one or more of physiological sensors (including wearable devices, such as armbands, wrist bands, chest bands, glasses, clothing, and the like), sensors on various devices used by a user, ambient sensors, and the like. These may include sensing one or more of temperature, pressure, ambient lighting conditions, localized lighting conditions, lighting spectrum characteristics, humidity, UV light, sound, particles, pollutants, gases (e.g., oxygen, carbon dioxide, carbon monoxide and radon), radiation, location of objects or items, motion (e.g., speed, direction and/or acceleration). Where one or more wearable or physiological sensors are used, they may sense one or more of a person's temperature, blood pressure, heart rate, oxygen saturation, activity type, activity level, galvanic skin response, respiratory rate, cholesterol level (including HDL, LDL and triglyceride), hormone or adrenal levels (e.g., Cortisol, thyroid, adrenaline, melatonin, and others), histamine levels, immune system characteristics, blood alcohol levels, drug content, macro and micro nutrients, mood, emotional state, alertness, sleepiness, and the like.

In some embodiments, the platform may connect to or integrate with data sources of information about users, such as including social networks (Facebook™, LinkedIn™ Twitter™, and the like, sources of medical records (23&Me™ and the like), productivity, collaboration and/or calendaring software (Google™, Outlook™, scheduling apps and the like), information about web browsing and/or shopping activity, activity on media streaming services (Netflix™, Spotify™, YouTube™, Pandora™ and the like), health record information and other sources of insight about the preferences or characteristics of users of the space of a lighting installation, including psychographic, demographic and other characteristics.

In some embodiments, the platform may use information from sources that indicate patterns, such as patterns involving periods of time (daily patterns, weekly patterns, seasonal patterns, and the like), patterns involving cultural factors or norms (such as indicating usage patterns or preferences in different regions), patterns relating to personality and preferences, patterns relating to social groups (such as family and work group patterns), and the like. In embodiments, the platform may make use of the data harvested from various sources noted above to make recommendations and/or to optimize (such as automatically, under computer control) the design, ordering, fulfillment, deployment and/or operation of a lighting installation, such as based on understanding or prediction of user behavior. This may include recommendation or optimization relating to achieving optimal sleep time and duration, setting optimal mealtimes, satisfying natural light exposure requirements during the day, and maintaining tolerable artificial light exposure levels (such as during night time). In some embodiments, the platform may anticipate user needs and optimize the lighting installation to enhance productivity, alertness, emotional well-being, satisfaction, safety and/or sleep. In further embodiments, the platform may control one or more panel systems of the present disclosure in accordance with the user needs of the environment based on this information.

In some embodiments, the platform may store a space utilization data structure that indicates, over time, how people use the space of the lighting installation, such as indicating what hallways are more trafficked, and the like. This may inform understanding of a space, such as indicating what is an entry, what is a passage, what is a workspace, and the like, which may be used to suggest changes or updates to a lighting design. In embodiments, sensors may be used to collect and read where people have been in the space, such as using one or more video cameras, IR sensors, microwave sensors. LIDAR, ultrasound or the like. In embodiments, the platform may collect and read what adjustments people have made, such as task lamp activation and other activities that indicate how a lighting fixture is used by an individual in a space. By way of these examples, aggregate usage information may be used to optimize a lighting design and adjust other lighting designs. Based on these factors, a space may be dynamically adjusted, and the lighting model for an installation may be updated to reflect the actual installation.

In some embodiments, control capabilities of the panel systems may include dynamic configuration of control parameters, such as providing a dimming curve for a light source, including but not limited to a panel system of the present disclosure, that is customized to the preferences of a designer or other user. This may include a selection from one or more modes, such as ones described elsewhere herein that have desired effects on mood or aesthetic factors, that have desired health effects, that meet the functional requirements, or the like.

Bioactive thresholds may, in some instances, benefit from prolonged exposure to at least one of one of CSE and LRNE. In some instances a melanopic flux of at least 10:1 may be suitable, in other instances the melanopic flux may be 20:1, 50:1, 100:1, or a greater ratio. It will be appreciated in light of the disclosure that traditional systems simply adjust from a warm CCT to a cool CCT, which may only provide a 2:1 or 3:1 ratio of melanopic flux, which are below said threshold. In some implementations, the platform may include spectral tuning targets for panel systems of the present disclosure that may optimize this ratio based on local installation environments. These targets, in a first operational mode along with adjustments intensity of light (e.g., 4:1) may provide a higher ratio, such as a 10:1 ratio or greater, and thus provide greater melanopic flux ratios.

In a second operational mode and either in combination with the above mode or not, the platform may support an ability to shift the bias of light in a room. In embodiments, controlled variation of one or more panel systems of the present disclosure in a lighting environment can contribute to generating a lighting bias typical of being outside.

In some implementations, various other programmable modes may be provided, such as bioactive panel system settings where using different combinations of color light sources to achieve a given mixed color output may be optimized for efficacy, efficiency, color quality, health impact (e.g., circadian action and/or LRNE action), or to satisfy other requirements. In embodiments, the programmable modes may also include programmable dimming curves, color tuning curves, and the like (such as allowing various control interfaces, such as extra-low voltage (ELV) controllers or voltage-based dimmers to affect fixture colors, such as where a custom tuning curve provides a start point, an end point and a dimming and/or color tuning path in response to a level of dimming). In embodiments, programmable modes may use conventional tuning mechanisms, such as simple interpolation systems (which typically use two or three white color LEDs) are dimmable on a zero to ten-volt analog system, and have a second voltage-based input for adjusting the CCT of a fixture between warm and cool CCTs. The bioactive panel systems as described herein can provide for tunable ranges of color points at various x, y coordinates on the 1931 CIE chromaticity diagram. Because of the wide range of potential white or non-white colors produced by the panel systems, they may be controlled by the platform that may specify a particular x, y coordinate on the CIE diagram. Lighting control protocols like DMX™ and Dali 2.0™ may achieve this result.

In some implementations the control system described herein controls output of at least one CSE and LRNE. In some embodiments a programmable color curve for an LED driver may be input, such as through an interface of the platform, or through a desktop software interface, a mobile phone 3330, a tablet app, or the like, that enables a user to define a start and stop point to a color tuning curve and to specify how it will be controlled by a secondary input, such as a voltage-based input (e.g., a 0 to 10-volt input) to the fixture. These may include pre-defined curves, as well as the ability to set start, end, and waypoints to define custom curves. For example, an exemplary color curve can have a starting point around 8000K biased above the black body curve, with the color curve crossing the black body around 2700K, and finishing around 1800K below the black body curve. Similarly, another exemplary curve could be programmed such that the start was 4000K well above the black body, with the end being 4000K well below the black body. By way of these examples, any adjustment would be in hue only, not CCT. Further examples may include a curve that never produces a white color, such as starting in the purple and finishing in orange. In any of these cases, these curves may be programmed into panel systems via the interface of the platform, the desktop, mobile phone or tablet. In embodiments, the curves may be designed, saved, and then activated, such as using the secondary (supplemental) 0 to 10-volt input.

In some implementations, a three-channel warm dim operational mode may be used, such as that described more fully in U.S. Provisional Patent Application No. 62/712,182 filed Jul. 30, 2018, which is incorporated herein in its entirety for all purposes, for target applications where the “fully on” CCT falls between 3000K and 2500K. By way of these examples, as the fixture dims (via ELV control or in response to the 0 to 10-volt input) the CCT may be gradually decreased to between 2500K and 1800K. In certain embodiments, the hue adjustment may all occur below the black body curve. Alternative embodiments may use a cyan channel as described elsewhere herein, either long-blue-pumped cyan or short-blue-pumped cyan, and a red channel which may be LRNE with cyan pumped near infrared as described elsewhere herein, plus a 4000K white channel as described elsewhere herein to achieve a warm dimming operational mode that allows for adjustment both above and below the black body curve. In some embodiments of the three-channel warm dim mode, the white channel can have a color point within a 7-step MacAdam ellipse around any point on the black body locus having a correlated color temperature between about 3500K and about 6500K.

In some implementations, the panel systems of the present disclosure can include a 4-channel color system as described elsewhere herein and in U.S. Provisional Patent Application No. 62/757,672 filed Nov. 8, 2018, and U.S. Provisional Application No. 62/712,191 filed Jul. 30, 2018, the contents of which are incorporated by reference herein in their entirety as if fully set forth herein, includes 3000K to 1800K CCT white color points within its range, a programmable mode may be included within the driver that adjusts color with the dimming percentage as well. In some aspects, this may be similar to a conventional control mode, except that the color control would not be on the secondary 0 to 10-volt channel, but may be activated through the primary 0 to 10-volt input channel or ELV controller. In embodiments, the “starting” color point may be the one when the fixture was “fully on.” In embodiments, the “ending” color point may be the one where the fixture is maximally dimmed. It is thus possible to make full range color change, such as purple to orange, which is slaved to the 0 to 10-volt or ELV dimming signal.

In some implementations, an optimized mode may be provided. With a 4-channel color system, there are many ways to create a single x-y point on the CIE diagram. In embodiments, the maximally efficient mode may typically be one that uses the colors that have x, y coordinates closest to the target x, y coordinate. But for best color quality, utilizing a fourth channel (and thereby requiring more light from the color in the opposite “corner”) may help provide a desired spectral power distribution. For the maximum melatonin suppression (for systems hoping to mimic circadian lighting), a higher cyan channel content may be required for CCTs of 3500K and above and minimizing cyan and blue content below 3500K. It will be appreciated in light of the disclosure that conventional systems either require expert users to understand the color balances necessary to achieve these effects (who then implement the color balances channel-by-channel) or are designed for maximum efficiency with color quality as a byproduct.

In some implementations, a digital power system is provided herein (including firmware-driven power conversion and LED current control) that controls a multichannel color system, such as a 4-channel color system, and allows for the inclusion of “modes” which may calculate the correct color balance between the various channels to provide optimized outputs. In embodiments, optimization may occur around one or more of efficacy, color quality, circadian effects, LRNE effects, and other factors. Other modes are possible, such as optimizing for contrast, particular display requirements. It will be appreciated in light of the disclosure that this is not an exhaustive list but is representative of potential modes that could be engaged through an interface of the platform (or of a mobile, tablet or desktop application) where a color tuning curve may be specified, such that the curve is used to specify an interface between a controller and the Digital PSU in a panel system. In embodiments, these modes may account for actual measured colors for each panel system and calculate the correct balance of for the chosen modes, such as based on algorithms loaded into the Digital PSU microprocessor.

In some implementations, machine learning may be used, such as based on various feedback measures, such as relating to mood (stated by the user or measured by one or more sensors), noise levels (such as indicating successful utilization of a space based on a desired level of noise), returns on investment (such as where panel systems are intended to promote retail merchandise), reported pain levels, measured health levels, performance levels of users (including fitness, wellness, and educational performance, among others), sleep levels, vitamin D levels, melatonin levels, and many others. In embodiments, the lighting installations including the panel systems may be operated or controlled based on external information, such as based on seasonal lighting conditions, weather, climate, collective mood indicators (such as based on stock market data, news feeds, or sentiment indices), analyses of social network data, and the like. This may include controlling a system to reflect, or influence, the mood of occupants.

FIG. 28 depicts an example computing environment 3000 suitable for implementing aspects of the embodiments of the present disclosure, including the control system, which can integrate one or more devices, computing, and lighting systems. As utilized herein, the phrase “computing system” generally refers to a dedicated computing device with processing power and storage memory, which supports operating software that underlies the execution of software, applications, and computer programs thereon. As used herein, an application is a small, in storage size, specialized program that is downloaded to the computing system or device. In some cases, the application is downloaded from an “App Store” such as APPLE's APP STORE or GOOGLE's ANDROID MARKET. After download, the application is generally installed on the computer system or computing device. As shown by FIG. 26, computing environment 3000 includes bus 3010 that directly or indirectly couples the following components: memory 3020, one or more processors 3030, I/O interface 3040, and network interface 3050. Bus 3010 is configured to communicate, transmit, and transfer data, controls, and commands between the various components of computing environment 3000.

Computing environment 3000 typically includes a variety of computer-readable media. Computer-readable media can be any available media that is accessible by computing environment 3000 and includes both volatile and nonvolatile media, removable and non-removable media. Computer-readable media may comprise both computer storage media and communication media. Computer storage media does not comprise, and in fact explicitly excludes, signals per se.

Computer storage media includes volatile and nonvolatile, removable and non-removable, tangible and non-transient media, implemented in any method or technology for storage of information such as computer-readable instructions, data structures, program modules or other data. Computer storage media includes RAM; ROM; EE-PROM; flash memory or other memory technology; CD-ROMs; DVDs or other optical disk storage; magnetic cassettes, magnetic tape, magnetic disk storage or other magnetic storage devices; or other mediums or computer storage devices which can be used to store the desired information and which can be accessed by computing environment 3000.

Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, communication media includes wired media, such as a wired network or direct-wired connection, and wireless media, such as acoustic, RF, infrared and other wireless media. Combinations of any of the above should also be included within the scope of computer-readable media.

Memory 3020 includes computer-storage media in the form of volatile and/or nonvolatile memory. The memory may be removable, non-removable, or a combination thereof. Memory 3020 may be implemented using hardware devices such as solid-state memory, hard drives, optical-disc drives, and the like. Computing environment 3000 also includes one or more processors 3030 that read data from various entities such as memory 3020, I/O interface 3040, and network interface 3050.

I/O interface 3040 enables computing environment 3000 to communicate with different input devices and output devices. Examples of input devices include a keyboard, a pointing device, a touchpad, a touchscreen, a scanner, a microphone, a joystick, and the like. Examples of output devices include a display device, an audio device (e.g., speakers), a printer, and the like. These and other I/O devices are often connected to processor 3010 through a serial port interface that is coupled to the system bus, but may be connected by other interfaces, such as a parallel port, game port, or universal serial bus (USB). A display device can also be connected to the system bus via an interface, such as a video adapter which can be part of, or connected to, a graphics processor unit. I/O interface 3040 is configured to coordinate I/O traffic between memory 3020, the one or more processors 3030, network interface 3050, and any combination of input devices and/or output devices.

Network interface 3050 enables computing environment 3000 to exchange data with other computing devices via any suitable network. In a networked environment, program modules depicted relative to computing environment 3000, or portions thereof, may be stored in a remote memory storage device accessible via network interface 3050. It will be appreciated that the network connections shown are exemplary and other means of establishing a communications link between the computers may be used.

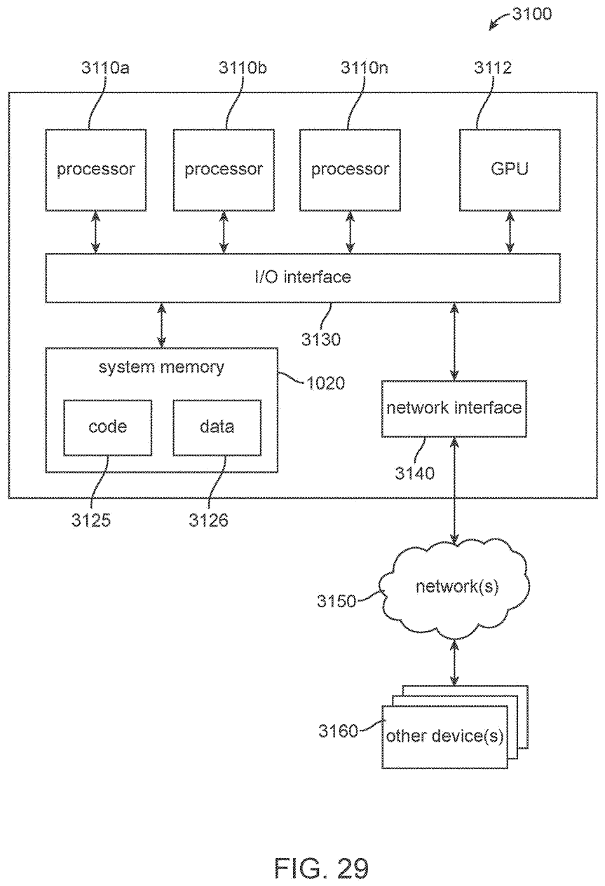

In at least some embodiments, a server that implements a portion or all of one or more of the technologies described herein may include a general-purpose computer system that includes or is configured to access one or more computer-accessible media. FIG. 29 depicts a general-purpose computer system that includes or is configured to access one or more computer-accessible media. In the illustrated embodiment, computing device 3100 includes one or more processors 3110a, 3110b, and/or 3110n (which may be referred herein singularly as a processor 1010 or in the plural as the processors 3110) coupled to a system memory 3120 via an input/output (“I/O”) interface 3130. Computing device 3100 further includes a network interface 3140 coupled to I/O interface 3130.

In various embodiments, computing device 3100 may be a uniprocessor system including one processor 3110 or a multiprocessor system including several processors 3110 (e.g., two, four, eight, or another suitable number). Processors 3110 may be any suitable processors capable of executing instructions. For example, in various embodiments, processors 3110 may be general-purpose or embedded processors implementing any of a variety of instruction set architectures (“ISAs”), such as the x86, PowerPC, SPARC or MIPS ISAs, or any other suitable ISA. In multiprocessor systems, each of processors 3110 may commonly, but not necessarily, implement the same ISA.

In some embodiments, a graphics processing unit (“GPU”) 3112 may participate in providing graphics rendering and/or physics processing capabilities. A GPU may, for example, comprise a highly parallelized processor architecture specialized for graphical computations. In some embodiments, processors 3110 and GPU 3112 may be implemented as one or more of the same type of device.

System memory 3120 may be configured to store instructions and data accessible by processor(s) 3110. In various embodiments, system memory 3120 may be implemented using any suitable memory technology, such as static random access memory (“SRAM”), synchronous dynamic RAM (“SDRAM”), nonvolatile/Flash®-type memory, or any other type of memory. In the illustrated embodiment, program instructions and data implementing one or more desired functions, such as those methods, techniques, and data described above, are shown stored within system memory 3120 as code 3125 and data 3126.

In one embodiment, I/O interface 3130 may be configured to coordinate I/O traffic between processor 3110, system memory 3120, and any peripherals in the device, including network interface 3140 or other peripheral interfaces. In some embodiments, I/O interface 3130 may perform any necessary protocol, timing or other data transformations to convert data signals from one component (e.g., system memory 3120) into a format suitable for use by another component (e.g., processor 3110). In some embodiments, I/O interface 3130 may include support for devices attached through various types of peripheral buses, such as a variant of the Peripheral Component Interconnect (“PCI”) bus standard or the Universal Serial Bus (“USB”) standard, for example. In some embodiments, the function of I/O interface 3130 may be split into two or more separate components, such as a north bridge and a south bridge, for example. Also, in some embodiments some or all of the functionality of I/O interface 3130, such as an interface to system memory 3120, may be incorporated directly into processor 3110.

Network interface 3140 may be configured to allow data to be exchanged between computing device 3100 and other device or devices 3160 attached to a network or networks 3150, such as other computer systems or devices, for example. In various embodiments, network interface 3140 may support communication via any suitable wired or wireless general data networks, such as types of Ethernet networks, for example. Additionally, network interface 3140 may support communication via telecommunications/telephony networks, such as analog voice networks or digital fiber communications networks, via storage area networks, such as Fibre Channel SANs (storage area networks), or via any other suitable type of network and/or protocol.

In some embodiments, system memory 3120 may be one embodiment of a computer-accessible medium configured to store program instructions and data as described above for implementing embodiments of the corresponding methods and apparatus. However, in other embodiments, program instructions and/or data may be received, sent, or stored upon different types of computer-accessible media. Generally speaking, a computer-accessible medium may include non-transitory storage media or memory media, such as magnetic or optical media, e.g., disk or DVD/CD coupled to computing device 3100 via I/O interface 3130. A non-transitory computer-accessible storage medium may also include any volatile or non-volatile media, such as RAM (e.g., SDRAM, DDR SDRAM, RDRAM, SRAM, etc.), ROM, etc., that may be included in some embodiments of computing device 3100 as system memory 3120 or another type of memory. Further, a computer-accessible medium may include transmission media or signals, such as electrical, electromagnetic or digital signals, conveyed via a communication medium, such as a network and/or a wireless link, such as those that may be implemented via network interface 3140. Portions or all of multiple computing devices, such as those illustrated in FIG. 28, may be used to implement the described functionality in various embodiments; for example, software components running on a variety of different devices and servers may collaborate to provide the functionality. In some embodiments, portions of the described functionality may be implemented using storage devices, network devices or special-purpose computer systems, in addition to or instead of being implemented using general-purpose computer systems. The term “computing device,” as used herein, refers to at least all these types of devices and is not limited to these types of devices.

A compute node, which may be referred to also as a computing node, may be implemented on a wide variety of computing environments, such as tablet computers, personal computers, smartphones, game consoles, commodity-hardware computers, virtual machines, web services, computing clusters, and computing appliances. Any of these computing devices or environments may, for convenience, be described as compute nodes or as computing nodes.

A network set up by an entity, such as a company or a public sector organization, to provide one or more web services (such as various types of cloud-based computing or storage) accessible via the Internet and/or other networks to a distributed set of clients may be termed a provider network. Such a provider network may include numerous data centers hosting various resource pools, such as collections of physical and/or virtualized computer servers, storage devices, networking equipment, and the like, needed to implement and distribute the infrastructure and web services offered by the provider network. The resources may in some embodiments be offered to clients in various units related to the web service, such as an amount of storage capacity for storage, processing capability for processing, as instances, as sets of related services, and the like. A virtual computing instance may, for example, comprise one or more servers with a specified computational capacity (which may be specified by indicating the type and number of CPUs, the main memory size, and so on) and a specified software stack (e.g., a particular version of an operating system, which may in turn run on top of a hypervisor).

A number of different types of computing devices may be used singly or in combination to implement the resources of the provider network in different embodiments, including general-purpose or special-purpose computer servers, storage devices, network devices, and the like. In some embodiments a client or user may be provided direct access to a resource instance, e.g., by giving a user an administrator login and password. In other embodiments the provider network operator may allow clients to specify execution requirements for specified client applications and schedule execution of the applications on behalf of the client on execution platforms (such as application server instances, Java™ virtual machines (“JVMs”), general-purpose or special-purpose operating systems, platforms that support various interpreted or compiled programming languages, such as Ruby, Perl, Python, C, C++, and the like, or high-performance computing platforms) suitable for the applications, without, for example, requiring the client to access an instance or an execution platform directly. A given execution platform may utilize one or more resource instances in some implementations; in other implementations multiple execution platforms may be mapped to a single resource instance.

In many environments, operators of provider networks that implement different types of virtualized computing, storage and/or other network-accessible functionality may allow customers to reserve or purchase access to resources in various resource acquisition modes. The computing resource provider may provide facilities for customers to select and launch the desired computing resources, deploy application components to the computing resources, and maintain an application executing in the environment. In addition, the computing resource provider may provide further facilities for the customer to quickly and easily scale up or scale down the numbers and types of resources allocated to the application, either manually or through automatic scaling, as demand for or capacity requirements of the application change. The computing resources provided by the computing resource provider may be made available in discrete units, which may be referred to as instances. An instance may represent a physical server hardware platform, a virtual machine instance executing on a server, or some combination of the two. Various types and configurations of instances may be made available, including different sizes of resources executing different operating systems (“OS”) and/or hypervisors, and with various installed software applications, runtimes, and the like. Instances may further be available in specific availability zones, representing a logical region, a fault tolerant region, a data center, or other geographic location of the underlying computing hardware, for example. Instances may be copied within an availability zone or across availability zones to improve the redundancy of the instance, and instances may be migrated within a particular availability zone or across availability zones. As one example, the latency for client communications with a particular server in an availability zone may be less than the latency for client communications with a different server. As such, an instance may be migrated from the higher latency server to the lower latency server to improve the overall client experience.

In some embodiments the provider network may be organized into a plurality of geographical regions, and each region may include one or more availability zones. An availability zone (which may also be referred to as an availability container) in turn may comprise one or more distinct locations or data centers, configured in such a way that the resources in a given availability zone may be isolated or insulated from failures in other availability zones. That is, a failure in one availability zone may not be expected to result in a failure in any other availability zone. Thus, the availability profile of a resource instance is intended to be independent of the availability profile of a resource instance in a different availability zone. Clients may be able to protect their applications from failures at a single location by launching multiple application instances in respective availability zones. At the same time, in some implementations inexpensive and low latency network connectivity may be provided between resource instances that reside within the same geographical region (and network transmissions between resources of the same availability zone may be even faster).

Each of the processes, methods, and algorithms described in the preceding sections may be embodied in, and fully or partially automated by, code modules executed by one or more computers or computer processors. The code modules may be stored on any type of non-transitory computer-readable medium or computer storage device, such as hard drives, solid state memory, optical disc, and/or the like. The processes and algorithms may be implemented partially or wholly in application-specific circuitry. The results of the disclosed processes and process steps may be stored, persistently or otherwise, in any type of non-transitory computer storage, such as, e.g., volatile or non-volatile storage.

The various features and processes described above may be used independently of one another, or may be combined in various ways. All possible combinations and sub-combinations are intended to fall within the scope of this disclosure. In addition, certain methods or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

It will also be appreciated that various items are illustrated as being stored in memory or on storage while being used, and that these items or portions thereof may be transferred between memory and other storage devices for purposes of memory management and data integrity. Alternatively, in other embodiments some or all of the software modules and/or systems may execute in memory on another device and communicate with the illustrated computing systems via inter-computer communication. Furthermore, in some embodiments, some or all of the systems and/or modules may be implemented or provided in other ways, such as at least partially in firmware and/or hardware, including, but not limited to, one or more application-specific integrated circuits (“ASICs”), standard integrated circuits, controllers (e.g., by executing appropriate instructions, and including microcontrollers and/or embedded controllers), field-programmable gate arrays (“FPGAs”), complex programmable logic devices (“CPLDs”), etc. Some or all of the modules, systems, and data structures may also be stored (e.g., as software instructions or structured data) on a computer-readable medium, such as a hard disk, a memory, a network, or a portable media article to be read by an appropriate device or via an appropriate connection. The systems, modules, and data structures may also be transmitted as generated data signals (e.g., as part of a carrier wave or other analog or digital propagated signal) on a variety of computer-readable transmission media, including wireless-based and wired/cable-based media, and may take a variety of forms (e.g., as part of a single or multiplexed analog signal, or as multiple discrete digital packets or frames). Such computer program products may also take other forms in other embodiments. Accordingly, the present invention may be practiced with other computer system configurations.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements, and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

While certain example embodiments have been described, these embodiments have been presented by way of example only, and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions and changes in the form of the methods and systems described herein may be made without departing from the spirit of the inventions disclosed herein. The accompanying claims and their equivalents are intended to cover such forms or modifications as would fall within the scope and spirit of certain of the inventions disclosed herein.

Those of ordinary skill in the art will appreciate that a variety of materials can be used in the manufacturing of the components in the devices and systems disclosed herein. Any suitable structure and/or material can be used for the various features described herein, and a skilled artisan will be able to select an appropriate structures and materials based on various considerations, including the intended use of the systems disclosed herein, the intended arena within which they will be used, and the equipment and/or accessories with which they are intended to be used, among other considerations. Conventional polymeric, metal-polymer composites, ceramics, and metal materials are suitable for use in the various components. Materials hereinafter discovered and/or developed that are determined to be suitable for use in the features and elements described herein would also be considered acceptable.

When ranges are used herein for physical properties, such as molecular weight, or chemical properties, such as chemical formulae, all combinations, and subcombinations of ranges for specific exemplar therein are intended to be included.

The disclosures of each patent, patent application, and publication cited or described in this document are hereby incorporated herein by reference, in its entirety.

Those of ordinary skill in the art will appreciate that numerous changes and modifications can be made to the exemplars of the disclosure and that such changes and modifications can be made without departing from the spirit of the disclosure. It is, therefore, intended that the appended claims cover all such equivalent variations as fall within the true spirit and scope of the disclosure.

What is claimed is:

1. A bioactive lighting system comprising:
a first lighting channel configured to produce a first white light having a first color point and a first spectral power distribution;
a second lighting channel configured to produce a second white light having a second color point and a second spectral power distribution;
a third lighting channel configured to produce a third white light having a third color point and a third spectral power distribution; and
a control system configured to independently change the intensity of each of the first, second and third lighting channel;
wherein at least two of the channels combined together to form a light effecting bio-physiological functions;
wherein the control system is further configured to change the intensity of each of the lighting channels to provide light with a color point at a plurality of points along a predefined path near the black body locus in the 1931 CIE Chromaticity Diagram; and,
wherein the first spectral power distribution has a first circadian-stimulating energy (CSE) characteristic, the second spectral power distribution has a second circadian-stimulating energy (CSE) characteristic, and the third spectral power distribution has a long red near infrared energy (LRNE) energy characteristic.

2-22. (canceled)
